(19) United States
(12) Patent Application Publication
(10) Pub. No.: US 2026/0085069 A1
(43) Pub. Date: Mar. 26, 2026

(54) PRODRUGS

(71) Applicant: Cambridge Enterprise Limited, Cambridge (GB)

(72) Inventor: PAUL GLOSSOP, Cambridgeshire (GB)

(21) Appl. No.: 19/109,796

(22) PCT Filed: Sep. 8, 2023
(86) PCT No.: PCT/GB2023/052328

§ 371 Date: Mar. 7, 2025

(30) Foreign Application Priority Data
Sep. 8, 2022 (GB) 2213162.7

(51) Int. Cl. C07D 471/04; A61K 31/4375

(52) U.S. Cl. CPC C07D 471/04; A61K 31/4375

(57) ABSTRACT

Provided are compounds of the Formula II, and salts, hydrates and solvates thereof:

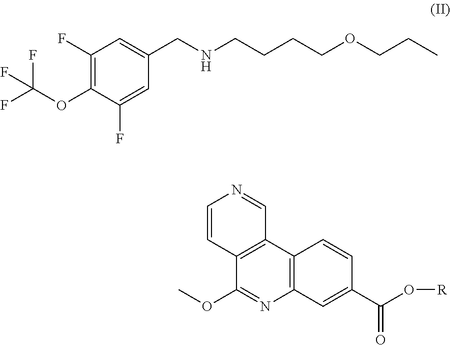

wherein R is as defined in the specification. The compounds are prodrugs of inhibitors of Casein Kinase 2 alpha (CK2α) and are useful for the treatment and/or prevention of diseases and conditions in which CK2α activity is implicated, such as, for example, but not limited to, the treatment and/or prevention of proliferative disorders (e.g. cancer), viral infections, inflammation, diabetes, vascular and ischemic disorders, neurodegeneration and the regulation of circadian rhythm. The present invention also relates to pharmaceutical compositions comprising the prodrugs defined herein and to their use for the treatment of diseases and/or conditions in which CK2α activity is implicated.

### INTRODUCTION

The present invention relates to prodrugs of compounds that inhibit Casein Kinase 2 alpha subunit (CK2α (CSNK2A1) and/or CK2α′ (CSNK2A2)) and as part of the CK2 holoenzyme. The prodrugs are therefore useful for the treatment and/or prevention of diseases and conditions in which CK2α activity is implicated, such as, for example but not limited to, the treatment and/or prevention of proliferative disorders (e.g. cancer), viral infections, inflammation, diabetes, vascular and ischemic disorders, neurodegeneration and the regulation of circadian rhythm.

The present invention also relates to pharmaceutical compositions comprising the prodrugs defined herein, to processes for synthesising these compounds and to their use for the treatment of diseases and/or conditions in which CK2α activity is implicated.

### BACKGROUND OF THE INVENTION

CK2α is a serine/threonine kinase that is a key regulator of many cellular processes and is involved in cellular proliferation and anti-apoptotic mechanisms (Battistutta & Lolli, Mol. Cell. Biochem. 2011). It mainly exists as a holoenzyme composed of two catalytic (a and/or α′) and a dimer of regulatory (β) subunits, but it can also be found as the isolated subunits (Niefind et al, EMBO J 2001). Unlike most other kinases, it is constitutively active and more than 300 proteins have been identified as putative CK2α substrates, making it one of the most pleiotropic proteins in eukaryotic systems (Meggio & Pinna, FASEB 2003).

CK2α is a pro-survival kinase that operates across multiple signaling pathways to convey a proliferative and anti-apoptotic phenotype to cells. Consequently, cancer cells are often described as being addicted to CK2α activity and a high-profile genome-wide CRISPR-Cas9 screen highlighted CK2α as a top tier, high priority drug target for Colorectal Cancer (CRC) (Behan et al, Nature 2019). The target is well validated by human data that correlates poor patient survival in numerous tumor types, including CRC, with increased CK2α expression (Lin et al, PLoS ONE 2011). Additionally, data from clinical samples shows CK2α expression is upregulated in numerous tumor types (Ortega et al, PLoS ONE 2014; Di Maira et al, 2019).

The human genetics of CRC are well characterized and approximately 80% tumors are identified as being wnt pathway mutation driven (e.g. APC, β-catenin) (Zhan et al, Oncogene 2017). The wnt pathway is known to be sensitive to and amplified by CK2α activity and can be inhibited by loss of CK2α function (Gao & Wang, J B C 2006). For example, in animal models, CK2α inhibition prevents tumor growth that is driven by different mutations in the wnt pathway (Dowling et al, ACS 2016).

CK2α also contributes to the malignant phenotype in cholangiocarcinoma (CCA), which is known to be a wnt-dysregulated tumor type (Zhan et al, Oncogene 2017). CK2α is over-expressed in human CCA samples and CCA tumor cell lines (Di Maira et al, Oncogenesis 2019); and disruption of CK2α activity in CCA cell models is reported to inhibit tumorigenic properties (Zakharia et al, Translational Oncology 2019).

It is hypothesised that a CK2α inhibitor given either as a monotherapy, in combination with standard of care chemotherapy or in combination with other targeted therapies in development, such as, but not limited to, KRAS inhibitors, will inhibit CRC tumor growth by reversing aberrant upregulation of wnt signaling to restore the normal balance of apoptosis and proliferation.

Existing CK2α inhibitors target the highly conserved ATP binding site. This design strategy often leads to a poor selectivity profile for such inhibitors over other kinases. There is therefore a need for potent and more selective CK2α inhibitors, including suitable prodrug forms of such inhibitors, that bind to the catalytic ATP site of CK2α (to drive potent enzyme inhibition) but also interact with other areas of CK2α, such as the αD site (to drive high levels of selectivity over other kinases). In particular, there is a need for prodrugs of CK2α inhibitors that have improved solubility.

The present invention was devised with the foregoing in mind.

### SUMMARY OF THE INVENTION

In one aspect, the present invention provides a compound of Formulae I or II as defined herein, and/or a pharmaceutically acceptable salt, hydrate or solvate thereof.

In another aspect, the present invention provides a pharmaceutical composition which comprises a compound of Formulae I or II as defined herein, or a pharmaceutically acceptable salt, hydrate or solvate thereof, and one or more pharmaceutically acceptable excipients.

In another aspect, the present invention provides a compound of Formulae I or II as defined herein, or a pharmaceutically acceptable salt, hydrate or solvate thereof, or a pharmaceutical composition as defined herein, for use in therapy.

In another aspect, the present invention provides a compound of Formulae I or II as defined herein, or a pharmaceutically acceptable salt, hydrate or solvate thereof, or a pharmaceutical composition as defined herein, for use in the treatment of a disease or condition in which CK2α activity is implicated.

In another aspect, the present invention provides a compound of Formulae I or II as defined herein, or a pharmaceutically acceptable salt, hydrate or solvate thereof, or a pharmaceutical composition as defined herein, for use in the treatment of a disease or condition associated with aberrant activity of CK2α.

In another aspect, the present invention provides a compound of Formulae I or II as defined herein, or a pharmaceutically acceptable salt, hydrate or solvate thereof, or a pharmaceutical composition as defined herein, for use in the treatment of proliferative disorders (e.g. cancer or benign neoplasms), viral infections, an inflammatory disease or condition, diabetes, vascular and ischemic disorders, neurodegenerative disorders and/or the regulation of circadian rhythm.

In another aspect, the present invention provides a compound of Formulae I or II as defined herein, or a pharmaceutically acceptable salt, hydrate or solvate thereof, or a pharmaceutical composition as defined herein, for use in the treatment of a cancer.

In another aspect, the present invention provides a compound of Formulae I or II as defined herein, or a pharmaceutically acceptable salt, hydrate or solvate thereof, or a pharmaceutical composition as defined herein, for use in the treatment of a viral infection.

In another aspect, the present invention provides the use of a compound of Formulae I or II as defined herein, or a pharmaceutically acceptable salt, hydrate or solvate thereof, in the manufacture of a medicament for use in the treatment of a disease or condition in which CK2α activity is implicated.

In another aspect, the present invention provides the use of a compound of Formulae I or II as defined herein, or a pharmaceutically acceptable salt, hydrate or solvate thereof, in the manufacture of a medicament for use in the treatment of a disease or condition associated with aberrant activity of CK2α.

In another aspect, the present invention provides the use of a compound of Formulae I or II as defined herein, or a pharmaceutically acceptable salt, hydrate or solvate thereof, in the manufacture of a medicament for use in the treatment of proliferative disorders (e.g. cancer or benign neoplasms), viral infections, an inflammatory disease or condition, diabetes, vascular and ischemic disorders, neurodegenerative disorders and/or the regulation of circadian rhythm.

In another aspect, the present invention provides the use of a compound of Formulae I or II as defined herein, or a pharmaceutically acceptable salt, hydrate or solvate thereof, in the manufacture of a medicament for use in the treatment of a cancer.

In another aspect, the present invention provides the use of a compound of Formulae I or II as defined herein, or a pharmaceutically acceptable salt, hydrate or solvate thereof, in the manufacture of a medicament for use in the treatment of a viral infection.

In another aspect, the present invention provides a method of treating a disease or condition in which CK2α activity is implicated, said method comprising administering to a subject in need thereof an effective amount of a compound of Formulae I or II as defined herein, or a pharmaceutically acceptable salt, hydrate or solvate thereof, or a pharmaceutical composition as defined herein.

In another aspect, the present invention provides a method of treating a disease or condition associated with aberrant activity of CK2α, said method comprising administering to a subject in need thereof an effective amount of a compound of Formulae I or II as defined herein, or a pharmaceutically acceptable salt, hydrate or solvate thereof, or a pharmaceutical composition as defined herein.

In another aspect, the present invention provides a method of treating a proliferative disorder (e.g. cancer or benign neoplasms), a viral infection, an inflammatory disease or condition, diabetes, vascular and ischemic disorders, neurodegenerative disorders and/or regulating cardiac rhythm, said method comprising administering to a subject in need thereof an effective amount of a compound of Formulae I or II as defined herein, or a pharmaceutically acceptable salt, hydrate or solvate thereof, or a pharmaceutical composition as defined herein.

In another aspect, the present invention provides a method of treating cancer, said method comprising administering to a subject in need thereof an effective amount of a compound of Formulae I or II as defined herein, or a pharmaceutically acceptable salt, hydrate or solvate thereof, or a pharmaceutical composition as defined herein.

In another aspect, the present invention provides a method of treating a viral infection, said method comprising administering to a subject in need thereof an effective amount of a compound of Formulae I or II as defined herein, or a pharmaceutically acceptable salt, hydrate or solvate thereof, or a pharmaceutical composition as defined herein.

In another aspect, the present invention provides a combination treatment comprising a compound of Formulae I or II, or a pharmaceutically acceptable salt, hydrate or solvate thereof, as defined herein, with one or more additional therapeutic agents.

In another aspect, the present invention provides processes for preparing compounds of Formulae I or II as defined herein, or a pharmaceutically acceptable salt, hydrate or solvate thereof, as defined herein, with one or more additional therapeutic agents.

Preferred, suitable, and optional features of any one particular aspect of the present invention are also preferred, suitable, and optional features of any other aspect.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

References to “Casein Kinase 2 alpha” or “CK2α” herein include CK2α (CSNK2A1) and/or CK2α′ (CSNK2A2). Where reference is made to the compounds of the present invention defined herein inhibiting CK2α or being CK2α inhibitors, we mean that the compounds function as inhibitors of CK2α (CSNK2A1) and/or CK2α′ (CSNK2A2) and the CK2 holoenzyme. In a particular embodiment, the compounds of the invention inhibit CK2α (CSNK2A1). In another embodiment, the compounds of the invention inhibit CK2α′ (CSNK2A2).

The compounds and intermediates described herein may be named according to either the IUPAC (International Union for Pure and Applied Chemistry) or CAS (Chemical Abstracts Service) nomenclature systems. It should be understood that unless expressly stated to the contrary, the terms “compounds of Formulae I and/or II”, “compounds of the invention” and the more general term “compounds” refer to and include any and all compounds described by and/or with reference to Formulae I and II herein. It should also be understood that these terms encompass all stereoisomers, i.e. cis and trans isomers, as well as optical isomers, i.e. R and S enantiomers, of such compounds, in substantially pure form and/or any mixtures of the foregoing in any ratio. This understanding extends to pharmaceutical compositions and methods of treatment that employ or comprise one or more compounds of the Formulae I or II, either by themselves or in combination with additional agents.

Unless specified otherwise, atoms are referred to herein by their chemical symbol as appearing in the IUPAC periodic table of the Elements. For example, “C” refers to a carbon atom.

The term “(m-nC)” or “(m-nC) group” used alone or as a prefix, refers to any group having m to n carbon atoms.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For Example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

An “alkylene” group is an alkyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene, ethylene, propylene, 2-methylpropylene, pentylene, and the like.

“(3-6C)cycloalkyl” means a hydrocarbon ring containing from 3 to 6 carbon atoms, for example, cyclopropyl, cyclobutyl, cyclopentyl or cyclohexyl.

The term “halo”, “halogen” or “halogeno” refers to fluoro, chloro, bromo and iodo.

As used herein, “oxo” refers to a double bond to oxygen, i.e. ═O.

As used herein by themselves or in conjunction with another term or terms, “haloalkyl” and “haloalkyl group” refer to alkyl groups in which one or more hydrogen atoms are replaced by halogen atoms. Representative examples include, but are not limited to, —CF₃, —CHF₂, —CH₂F, —CF₂CF₃, —CHFCF₃, and —CH₂CF₃. Suitably, a haloalkyl group is selected from —CHF₂ and —CF₃, suitably —CF₃.

As used herein by themselves or in conjunction with another term or terms, “haloalkoxy” and “haloalkoxy group” refer to alkoxy groups (i.e. O-alkyl groups) in which one or more hydrogen atoms are replaced by halogen atoms. Representative examples include, but are not limited to, —OCF₃, —OCHF₂, —OCH₂F, and —OCF₂CF₃. Suitably, a haloalkoxy group is selected from —OCHF₂ and —OCF₃, suitably —OCF₃.

The term “heterocyclyl”, “heterocyclic” or “heterocycle” means a non-aromatic saturated or partially saturated (i.e. can include one or more unsaturated bonds) monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as, but not limited to, oxiranyl, oxetanyl, tetrahydrofuranyl, dioxanyl, dioxoles (e.g. 1,2-dioxole and 1,3-dioxole) and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydrooxathiolyl, tetrahydrooxazolyl, tetrahydro-oxadiazolyl, tetrahydrod ioxazolyl, tetrahydrooxathiazolyl, hexahydrotriazinyl, tetrahydrooxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as, but not limited to, tetrahydrothiene 1,1-dioxide and thiomorpholinyl 1,1-dioxide. A suitable value for a heterocyclyl group which bears 1 or 2 oxo (=O) or thioxo (=S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

By “spiro bicyclic ring systems” we mean that the two ring systems share one common spiro carbon atom, i.e. the heterocyclic ring is linked to a further carbocyclic or heterocyclic ring through a single common spiro carbon atom. Examples of spiro ring systems include 6-azaspiro[3.4]octane, 2-oxa-6-azaspiro[3.4]octane, 2-azaspiro[3.3]heptanes, 2-oxa-6-azaspiro[3.3]heptanes, 7-oxa-2-azaspiro[3.5]nonane, 6-oxa-2-azaspiro[3.4]octane, 2-oxa-7-azaspiro[3.5]nonane and 2-oxa-6-azaspiro[3.5]nonane.

The term “heteroaryl” or “heteroaromatic” means an aromatic mono-, bi-, or polycyclic ring incorporating one or more (for example 14, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically, the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3b]-furanyl-, 2H-furo[3,2b]-pyranyl-, 5H-pyrido[2,3-d]-ooxazinyl-, 1H-pyrazolo[4,3-d]-oxazolyl, 4H-imidazo[4,5d]thiazolyl, pyrazino[2,3d]pyridazinyl, -imidazo[2,1b]thiazolyl, -imidazo[1,2b][1,2,4]-triazinyl. “Heteroaryl” also covers partially aromatic bi- or polycyclic ring systems wherein at least one ring is an aromatic ring and one or more of the other ring(s) is a nonaromatic, saturated or partially saturated ring, provided at least one ring contains one or more heteroatoms selected from nitrogen, oxygen or -sulfur-. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl, 3,4-dihydro-2H-pyrido[3,2-b][1,4]oxazinyl and 6,8-dihydro-5H-[1,2,4]triazolo[4,3-a]pyrazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:
a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms;
a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In particular embodiment, an aryl is phenyl.

Several composite terms may be used to describe groups comprising more than one functionality. Such terms will be understood by a person skilled in the art. For example, heterocyclyl(m-nC)alkyl comprises (m-nC)alkyl substituted by heterocyclyl.

The term “optionally substituted” refers to either groups, structures, or molecules that are substituted and those that are not substituted. The term “wherein R₂ is optionally substituted by one or more substituents independently selected from . . . ” suitably means that (any) one of the hydrogen radicals of the R₂ group is substituted by a relevant stipulated group.

Where optional substituents are chosen from “one or more” groups it is to be understood that this definition includes all substituents being chosen from one of the specified groups or the substituents being chosen from two or more of the specified groups.

A wavy bond ( [symbol] ) is used herein to show a point of attachment.

The phrase “compound of the invention” means those compounds which are disclosed herein, both generically and specifically.

As used herein by itself or in conjunction with another term or terms, “pharmaceutically acceptable” refers to materials that are generally chemically and/or physically compatible with other ingredients (such as, for example, with reference to a formulation), and/or are generally physiologically compatible with the recipient (such as, for example, a subject) thereof.

As used herein by themselves or in conjunction with another term or terms, “subject(s)” and “patient(s)”, suitably refer to mammals, in particular humans.

### Compounds of the Invention

In a first aspect, the present invention relates to a compound, or pharmaceutically acceptable salt, hydrate or solvate thereof, having the structural formula I shown below:

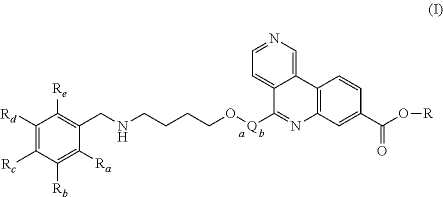

wherein:
   R is an in vivo cleavable ester group;
   Q is selected from formula Ia or Ib:

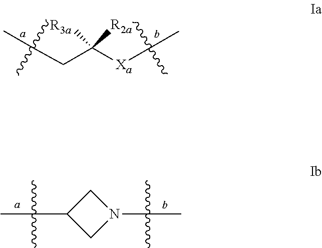

wherein:
bond a in formulae Ia and Ib corresponds with bond a in formula I and bond b in formulae Ia and Ib corresponds with bond b in formula I;
R₂ₐ and R₃ₐ are each independently selected from hydrogen or methyl; and
Xₐ is NH or O;
Rₐ and Rₑ are both independently selected from hydrogen, methyl or halo; R_{b} and R_{d} are each independently selected from hydrogen, halo, cyano, (1-4C)alkyl,
   —[CH₂]₀₋₃-(1-4C)alkoxy,
   —[CH₂]₀₋₃—C(O)NH₂,
   —[CH₂]₀₋₃—C(O)NH(1-4C)alkyl,
   —[CH₂]₀₋₃—C(O)N[(1-4C)alkyl]₂,
   —[CH₂]₀₋₃—NH(1-4C)alkyl,
   —[CH₂]₀₋₃—N[(1-4C)alkyl]₂,
   —[CH₂]₀₋₃—S(O)_{q}-(1-4C)alkyl (wherein q is 0, 1 or 2),
   —[CH₂]₀₋₃—C(O)(1-4C)alkyl,
   —[CH₂]₀₋₃—C(O)O-(1-4C)alkyl,
   —[CH₂]₀₋₃—N(R_{f})C(O)-(1-4C)alkyl (wherein R_{f} is hydrogen or methyl),
   —[CH₂]₀₋₃—S(O)₂NH(1-4C)alkyl,
   —[CH₂]₀₋₃—S(O)₂N[(1-4C)alkyl]₂,
   —[CH₂]₀₋₃—N(R)SO₂-(1-4C)alkyl (wherein R_{g} is hydrogen or methyl),
   a group of the formula:

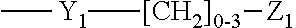

wherein Y₁ is absent, —O—, —NH—, —NMe—, —S—, —S(O)— or —S(O)₂—; and
Z₁ is (3-6C)cycloalkyl, phenyl, a 4- to 6-membered heterocyclyl or 5 or 6-membered heteroaryl;
and wherein:
any alkyl, alkoxy or any alkyl moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
R_{c} is selected from hydrogen, halo, cyano, —C(O)NH₂, (1-4C)alkyl,
   —[CH₂]₀₋₃-(1-4C)alkoxy,
   —[CH₂]₀₋₃-(3-6C)cycloalkoxy,
   —[CH₂]₀₋₃—C(O)NH₂,
   —[CH₂]₀₋₃—C(O)NH(1-4C)alkyl,
   —[CH₂]₀₋₃—C(O)N[(1-4C)alkyl]₂,
   —[CH₂]₀₋₃—NH(1-4C)alkyl,
   —[CH₂]₀₋₃—N[(1-4C)alkyl]₂,
   —[CH₂]₀₋₃—S(O)_{q}-(1-4C)alkyl (wherein q is 0, 1 or 2),
   —[CH₂]₀₋₃—C(O)(1-4C)alkyl,
   —[CH₂]₀₋₃—C(O)O-(1-4C)alkyl,
   —[CH₂]₀₋₃—N(Rₕ)C(O)-(1-4C)alkyl (wherein Rₕ is hydrogen or methyl),
   —[CH₂]₀₋₃—S(O)₂NH(1-4C)alkyl,
   —[CH₂]₀₋₃—S(O)₂N[(1-4C)alkyl]₂,
   —[CH₂]₀₋₃—N(Rᵢ)SO₂-(1-4C)alkyl (wherein Rᵢ is hydrogen or methyl),
   a group of the formula:

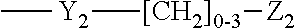

wherein Y₂ is absent, —O—, —NH—, —NMe—, —S—, —S(O)— or —S(O)₂—; and
Z₂ is (3-6C)cycloalkyl, phenyl, a 4- to 6-membered heterocyclyl or 5 or 6-membered heteroaryl;
and wherein:
   any alkyl, alkoxy or any alkyl moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and
   Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl.

Suitably, in any of the definitions of formula I set out herein, R is selected from -(1-6C)alkyl, -(1-4C)alkylene-X—R₁, and -(1-4C)alkylene-R₂, wherein:
X is selected from —O—, —C(O)—, —C(O)O—, —O—C(O)—, —O—C(O)—O—, —CH(OR^{A})—, —N(R^{A})—, —N(R^{A})—C(O)—, —N(R^{A})—C(O)O—, —C(O)—N(R^{A})—, —N(R^{A})C(O)N(R^{A})—, —S—, —SO—, —SO₂—, —S(O)₂N(R^{A})—, or —N(R^{A})SO₂— wherein each R^{A} is independently selected from hydrogen or (1-6C)alkyl;
R₁ is selected from -hydrogen, -(1-6C)alkyl, -(2-6C)alkenyl, -(2-6C)alkynyl, -aryl, -(3-6C)cycloalkyl, -(3-6C)cycloalkenyl, -heteroaryl or -heterocyclyl; and
R₂ is selected from -aryl, -(3-6C)cycloalkyl, -(3-6C)cycloalkenyl, -heteroaryl or -heterocyclyl, wherein R₂ is optionally substituted by one or more substituents independently selected from -halo, -hydroxy, -cyano, -amino, -(1-4C)alkyl, -(1-4C)alkoxy, -oxo, —C(O)—R^{B}, —C(O)O—R^{B} or —C(O)—N(R^{B})₂, wherein each R^{B} is independently selected from hydrogen or (1-6C)alkyl.

In a particular group of compounds of the invention, the compounds have the structural formula I shown above, with the proviso that the compound is not:
(S)-Methyl 5-((1-(4-((3-chloro-4-(trifluoromethoxy)benzyl)amino)butoxy)propan-2-yl)oxy)benzo[c][2,6]naphthyridine-8-carboxylate

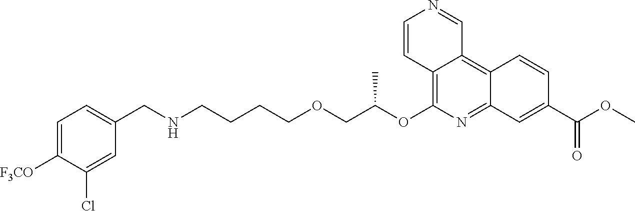 [SMILES: COC(=O)c1ccc2c(c1)nc(O[C@@H](C)COCCCCNCc1ccc(OC(F)(F)F)c(Cl)c1)c1ccncc12]

In a particular group of compounds of the invention, there is provided a compound, or pharmaceutically acceptable salt, hydrate or solvate thereof, having the structural formula II shown below:

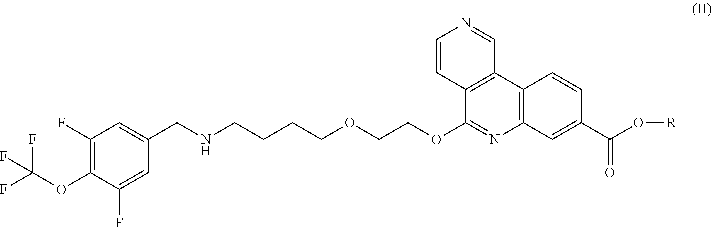

wherein R is an in vivo cleavable ester group.

Suitably, in any of the definitions of formula II set out herein, R is selected from: -(1-6C)alkyl, -(1-4C)alkylene-X—R, and -(1-4C)alkylene-R₂, wherein:
X is selected from —O—, —C(O)—, —C(O)O—, —O—C(O)—, —O—C(O)—O—, —CH(OR^{A})—, —N(R^{A})—, —N(R^{A})—C(O)—, —N(R^{A})—C(O)O—, —C(O)—N(R^{A})—, —N(R^{A})C(O)N(R^{A})—, —S—, —SO—, —SO₂—, —S(O)₂N(R^{A})—, or —N(R^{A})SO₂— wherein each R^{A} is independently selected from hydrogen or (1-6C)alkyl;
R₁ is selected from -hydrogen, -(1-6C)alkyl, -(2-6C)alkenyl, -(2-6C)alkynyl, -aryl, -(3-6C)cycloalkyl, -(3-6C)cycloalkenyl, -heteroaryl or -heterocyclyl; and
R₂ is selected from -aryl, -(3-6C)cycloalkyl, -(3-6C)cycloalkenyl, -heteroaryl or -heterocyclyl, wherein R₂ is optionally substituted by one or more substituents independently selected from -halo, -hydroxy, -cyano, -amino, -(1-4C)alkyl, -(1-4C)alkoxy, -oxo, —C(O)—R^{B}, —C(O)O—R^{B} or —C(O)—N(R^{B})₂, wherein each R^{B} is independently selected from hydrogen or (1-6C)alkyl.

Particular compounds of the invention include, for example, compounds of the formulae I or II, or pharmaceutically acceptable salts, hydrates and/or solvates thereof, wherein, unless otherwise stated, R, and any group associated therewith, have any of the meanings defined hereinbefore or are as defined in any one of paragraphs (1) to (29) hereinafter:—
(1) R is selected from -(1-6C)alkyl, -(1-4C)alkylene-X—R₁, and -(1-4C)alkylene-R₂;
(2) R is selected from -(1-5C)alkyl, -(1-3C)alkylene-X—R₁, and -(1-3C)alkylene-R₂,
(3) R is selected from -(1-4C)alkyl, -(1-2C)alkylene-X—R₁, and -(1-2C)alkylene-R₂,
(4) R is selected from -methyl, -ethyl, -propyl (e.g. n-propyl and iso-propyl), -butyl (e.g. n-butyl, iso-butyl, sec-butyl and tert-butyl), -methylene-X—R₁, -ethylene-X—R₁, -methylene-R₂ and -ethylene-R₂;
(5) R is selected from -methyl, -ethyl, -isopropyl, -tertbutyl, -methylene-X—R₁, -ethylene-X—R₁ and -methylene-R₂;
(6) X is selected from —O—, —C(O)—, —C(O)O—, —O—C(O)—, —O—C(O)—O—, —CH(OR^{A})—, —N(R^{A})—, —N(R^{A})—C(O)—, —N(R^{A})—C(O)O—, —C(O)—N(R^{A})—, —N(R^{A})C(O)N(R^{A})—, —S—, —SO—, —SO₂—, —S(O)₂N(R^{A})—, or —N(R^{A})SO₂— wherein each R^{A} is independently selected from hydrogen or (1-6C)alkyl;
(7) X is selected from —O—, —C(O)—, —C(O)O—, —O—C(O)—, —O—C(O)—O—, —CH(OR^{A})—, —N(R^{A})—, —N(R^{A})—C(O)—, —N(R^{A})—C(O)O—, —C(O)—N(R^{A})— or —N(R^{A})C(O)N(R^{A})—, wherein each R^{A} is independently selected from hydrogen or (1-4C)alkyl;
(8) X is selected from —O—, —C(O)—, —C(O)O—, —O—C(O)—, —O—C(O)—O—, —N(R^{A})—, —N(R^{A})—C(O)— or —C(O)—N(R^{A})—, wherein R^{A} is selected from hydrogen or (1-3C)alkyl;
(9) X is selected from —O—, —C(O)—, —C(O)O—, —O—C(O)—, —O—C(O)—O— or —N(R^{A})—wherein R^{A} is selected from hydrogen, methyl or ethyl;
(10) X is selected from —C(O)—, —C(O)O—, —O—C(O)—, —O—C(O)—O—, —N(H)— or —N(Me)—;
(11) X is selected from —O—C(O)—, —O—C(O)—O— or —N(Me)—;
(12) R₁ is selected from -hydrogen, -(1-6C)alkyl, -(2-6C)alkenyl, -(2-6C)alkynyl, -aryl, -(3-6C)cycloalkyl, -(3-6C)cycloalkenyl, -heteroaryl or -heterocyclyl;
(13) R₁ is selected from -hydrogen, -(1-5C)alkyl, -(2-5C)alkenyl, -(2-5C)alkynyl, -aryl, -(3-6C)cycloalkyl, -(3-6C)cycloalkenyl, -heteroaryl or -heterocyclyl;
(14) R₁ is selected from -hydrogen, -(1-5C)alkyl, -aryl, -(3-6C)cycloalkyl, -(3-6C)cycloalkenyl, -heteroaryl or -heterocyclyl;
(15) R₁ is selected from -hydrogen, -(1-4C)alkyl, -aryl, -(4-6C)cycloalkyl, -5- or -6-membered heteroaryl or -5- or -6-membered heterocyclyl, wherein said heteroaryl or heterocyclyl comprises 1, 2 or 3 heteroatoms selected from N, O and S;
(16) R₁ is selected from -hydrogen, -methyl, -ethyl, -propyl (e.g. n-propyl and iso-propyl), -butyl (e.g. n-butyl, iso-butyl, sec-butyl and tert-butyl), -phenyl, cyclopentane, cyclohexane, -5- or -6-membered heteroaryl or -5- or -6-membered heterocyclyl, wherein said heteroaryl or heterocyclyl comprises 1 or 2 heteroatoms selected from N, O and S;
(17) R₁ is selected from -hydrogen, -methyl, -ethyl, -propyl (e.g. n-propyl and iso-propyl), -butyl (e.g. n-butyl, iso-butyl, sec-butyl and tert-butyl) or -6-membered heterocyclyl, wherein said heterocyclyl comprises 1 or 2 heteroatoms selected from N and O;
(18) R₁ is selected from -methyl, -ethyl, -isopropyl, -tertbutyl or -tetrahydropyran;
(19) R₂ is selected from -aryl, -(3-6C)cycloalkyl, -(3-6C)cycloalkenyl, -heteroaryl or -heterocyclyl, wherein R₂ is optionally substituted by one or more substituents independently selected from -halo, -hydroxy, -cyano, -amino, -(1-4C)alkyl, -(1-4C)alkoxy, -oxo, —C(O)—R^{B}, —C(O)O—R^{B} or —C(O)—N(R^{B})₂, wherein each R^{B} is independently selected from hydrogen or (1-6C)alkyl;
(20) R₂ is selected from -aryl, -(4-6C)cycloalkyl, -(4-6C)cycloalkenyl, -heteroaryl or -heterocyclyl, wherein R₂ is optionally substituted by one or more substituents independently selected from -halo, -hydroxy, -cyano, -amino, -(1-3C)alkyl, -(1-3C)alkoxy, -oxo, —C(O)—R^{B}, —C(O)O—R^{B} or —C(O)—N(R^{B})₂, wherein each R^{B} is independently selected from hydrogen or (1-4C)alkyl;
(21) R₂ is selected from -phenyl, -(4-6C)cycloalkyl, -(4-6C)cycloalkenyl, -heteroaryl or -heterocyclyl, wherein said heteroaryl or heterocyclyl comprises 1, 2 or 3 heteroatoms selected from N, O and S, wherein R₂ is optionally substituted by one or more substituents independently selected from -halo, -hydroxy, -cyano, -amino, -(1-3C)alkyl, -(1-3C)alkoxy, -oxo, —C(O)—R^{B}, —C(O)O—R^{B} or —C(O)—N(R^{B})₂, wherein each R^{B} is independently selected from hydrogen or (1-4C)alkyl;
(22) R₂ is selected from -phenyl, -(4-6C)cycloalkyl, -(4-6C)cycloalkenyl, -5-, or -6-membered heteroaryl or -5-, or -6-membered heterocyclyl, wherein said heteroaryl or heterocyclyl comprises 1, 2 or 3 heteroatoms selected from N, O and S, wherein R₂ is optionally substituted by one or more substituents independently selected from -halo, -hydroxy, -cyano, -amino, -(1-3C)alkyl, -(1-3C)alkoxy, -oxo, —C(O)—R^{B}, —C(O)O—R^{B} or —C(O)—N(R^{B})₂, wherein each R^{B} is independently selected from hydrogen or (1-3C)alkyl;
(23) R₂ is selected from -phenyl, -(4-6C)cycloalkyl, -(4-6C)cycloalkenyl, -5-, or -6-membered heteroaryl or -5-, or -6-membered heterocyclyl, wherein said heteroaryl or heterocyclyl comprises 1 or 2 heteroatoms selected from N, O and S, wherein R₂ is optionally substituted by one, two or three substituents independently selected from -halo, -hydroxy, -cyano, -amino, -(1-3C)alkyl, -(1-3C)alkoxy, -oxo, —C(O)—R^{B}, —C(O)O—R^{B} or —C(O)—N(R^{B})₂, wherein each R^{B} is independently selected from hydrogen or (1-3C)alkyl;
(24) R₂ is selected from −5-, or -6-membered heteroaryl or -5-, or -6-membered heterocyclyl, wherein said heteroaryl or heterocyclyl comprises 1 or 2 heteroatoms selected from N and O, wherein R₂ is optionally substituted by one, two or three substituents independently selected from -halo, -hydroxy, -cyano, -amino, -(1-3C)alkyl, -(1-3C)alkoxy, -oxo, —C(O)—H, —C(O)O—H or —C(O)—N(H)₂, —C(O)—Me, —C(O)O-Me or —C(O)—N(Me)₂;
(25) R₂ is a -5-membered heterocyclyl, wherein said heterocyclyl comprises 2 heteroatoms selected from N and O, wherein R₂ is optionally substituted by one or two substituents independently selected from -halo, -hydroxy, -cyano, -amino, -(1-3C)alkyl, -(1-3C)alkoxy, -oxo, —C(O)—H, —C(O)O—H or —C(O)—N(H)₂, —C(O)—Me, —C(O)O-Me or —C(O)—N(Me)₂;
(26) R₂ is a dioxole (e.g. 1,2-dioxole or 1,3-dioxole), wherein R₂ is optionally substituted by two substituents independently selected from -halo, -hydroxy, -cyano, -amino, -(1-3C)alkyl, -(1-3C)alkoxy, -oxo, —C(O)—H, —C(O)O—H or —C(O)—N(H)₂, —C(O)—Me, —C(O)O-Me or —C(O)—N(Me)₂;
(27) R₂ is a dioxole (e.g. 1,2-dioxole or 1,3-dioxole), wherein R₂ is optionally substituted by two substituents independently selected from -halo, -hydroxy, -(1-3C)alkyl or -oxo;
(28) R₂ is a dioxole (e.g. 1,2-dioxole or 1,3-dioxole), wherein R₂ is substituted by two substituents independently selected from -methyl, -ethyl or -oxo;
(29) R₂ is 1,3-dioxole, wherein R₂ is substituted by one methyl group and one oxo group.

Suitably, in any of the definitions of formulae I or II set out herein, a heteroaryl is a 5- or 6-membered heteroaryl ring comprising one, two or three heteroatoms selected from N, O or S.

Suitably, in any of the definitions of formulae I or 11 set out herein, a heterocyclyl group is a 4-, 5- or 6-membered heterocyclyl ring comprising one, two or three heteroatoms selected from N, O or S. Most suitably, a heterocyclyl group is a 4-, 5- or 6-membered ring comprising one or two heteroatoms selected from N, O or S [e.g. morpholinyl (e.g. 4-morpholinyl), piperidinyl, piperazinyl or pyrrolidinyl].

Suitably, in any of the definitions of formulae I or 11 set out herein, R is as defined in any one of paragraphs (1) to (5) above. More suitably, R is as defined in any one of paragraphs paragraph (2) to (5) above. Even more suitably, R is as defined in any one of paragraphs (3), (4) or (5) above. Yet even more suitably, R is as defined in any one of paragraphs (4) or (5) above. Most suitably, R is as defined in paragraph (5) above.

In a particular group of compounds of formulae I or II, R is as defined in paragraph (1) above, and X, R₁ and R₂, and any group associated therewith, are each as defined herein.

In a particular group of compounds of formulae I or II, R is as defined in paragraph (2) above, and X, R₁ and R₂, and any group associated therewith, are each as defined herein.

In a particular group of compounds of formulae I or II, R is as defined in paragraph (3) above, and X, R₁ and R₂, and any group associated therewith, are each as defined herein.

In a particular group of compounds of formulae I or II, R is as defined in paragraph (4) above, and X, R₁ and R₂, and any group associated therewith, are each as defined herein.

In a particular group of compounds of formulae I or II, R is as defined in paragraph (5) above, and X, R₁ and R₂, and any group associated therewith, are each as defined herein.

Suitably, in any of the definitions of formulae I or II set out herein, X is as defined in any one of paragraphs (6) to (11) above. More suitably, X is as defined in any one of paragraphs (7) to (11) above. Even more suitably, X is as defined in any one of paragraphs (8), (9), (10) or (11) above. Yet more suitably, X is as defined in any one of paragraphs (9), (10) or (11) above. Yet even more suitably, X is as defined in any one of paragraphs (10) or (11) above. Most suitably, X is as defined in paragraph (11) above.

In a particular group of compounds of formulae I or II wherein R is -(1-4C)alkylene-X—R₁, or a sub-definition thereof, X is as defined in paragraph (6) above, and R₁ is as defined herein.

In a particular group of compounds of formulae I or II wherein R is -(1-4C)alkylene-X—R₁, or a sub-definition thereof, X is as defined in paragraph (7) above, and R₁ is as defined herein.

In a particular group of compounds of formulae I or II wherein R is -(1-4C)alkylene-X—R₁, or a sub-definition thereof, X is as defined in paragraph (8) above, and R₁ is as defined herein.

In a particular group of compounds of formulae I or II wherein R is -(1-4C)alkylene-X—R₁, or a sub-definition thereof, X is as defined in paragraph (9) above, and R₁ is as defined herein.

In a particular group of compounds of formulae I or II wherein R is -(1-4C)alkylene-X—R₁, or a sub-definition thereof, X is as defined in paragraph (10) above, and R₁ is as defined herein.

In a particular group of compounds of formulae I or II wherein R is -(1-4C)alkylene-X—R₁, or a sub-definition thereof, X is as defined in paragraph (11) above, and R₁ is as defined herein.

Suitably, in any of the definitions of formulae I or 11 set out herein, R₁ is as defined in any one of paragraphs (12) to (18) above. More suitably, R₁ is as defined in any one of paragraphs (13) to (18) above. Even more suitably, R₁ is as defined in any one of paragraphs (14) to (18) above. Yet more suitably, R₁ is as defined in any one of paragraphs (15), (16), (17) or (18) above. Yet even more suitably, R₁ is as defined in any one of paragraphs (16), (17), or (18) above. Most suitably, R₁ is as defined in any one of paragraphs (17) or (18) above.

In a particular group of compounds of formulae I or II wherein R is -(1-4C)alkylene-X—R₁, or a sub-definition thereof, R₁ is as defined in paragraph (12) above, and X, and any group associated therewith, is as defined herein.

In a particular group of compounds of formulae I or II wherein R is -(1-4C)alkylene-X—R₁, or a sub-definition thereof, R₁ is as defined in paragraph (13) above, and X, and any group associated therewith, is as defined herein.

In a particular group of compounds of formulae I or II wherein R is -(1-4C)alkylene-X—R₁, or a sub-definition thereof, R₁ is as defined in paragraph (14) above, and X, and any group associated therewith, is as defined herein.

In a particular group of compounds of formulae I or II wherein R is -(1-4C)alkylene-X—R₁, or a sub-definition thereof, R₁ is as defined in paragraph (15) above, and X, and any group associated therewith, is as defined herein.

In a particular group of compounds of formulae I or II wherein R is -(1-4C)alkylene-X—R₁, or a sub-definition thereof, R₁ is as defined in paragraph (16) above, and X, and any group associated therewith, is as defined herein.

In a particular group of compounds of formulae I or II wherein R is -(1-4C)alkylene-X—R₁, or a sub-definition thereof, R₁ is as defined in paragraph (17) above, and X, and any group associated therewith, is as defined herein.

In a particular group of compounds of formulae I or II wherein R is -(1-4C)alkylene-X—R₁, or a sub-definition thereof, R₁ is as defined in paragraph (18) above, and X, and any group associated therewith, is as defined herein.

In a particular group of compounds of formulae I or II wherein R is -(1-4C)alkylene-X—R₁, or a sub-definition thereof, X, and any group associated therewith, is as defined in paragraph (6) above and R₁ is as defined in paragraph (12) above.

In a particular group of compounds of formulae I or II wherein R is -(1-4C)alkylene-X—R₁, or a sub-definition thereof, X, and any group associated therewith, is as defined in paragraph (7) above and R₁ is as defined in paragraph (13) above.

In a particular group of compounds of formulae I or II wherein R is -(1-4C)alkylene-X—R₁, or a sub-definition thereof, X, and any group associated therewith, is as defined in paragraph (8) above and R₁ is as defined in paragraph (14) above.

In a particular group of compounds of formulae I or II wherein R is -(1-4C)alkylene-X—R₁, or a sub-definition thereof, X, and any group associated therewith, is as defined in paragraph (9) above and R₁ is as defined in paragraph (15) above.

In a particular group of compounds of formulae I or II wherein R is -(1-4C)alkylene-X—R₁, or a sub-definition thereof, X, and any group associated therewith, is as defined in paragraph (10) above and R₁ is as defined in paragraph (16) above.

In a particular group of compounds of formulae I or II wherein R is -(1-4C)alkylene-X—R₁, or a sub-definition thereof, X, and any group associated therewith, is as defined in paragraph (11) above and R₁ is as defined in paragraph (17) above.

In a particular group of compounds of formulae I or II wherein R is -(1-4C)alkylene-X—R₁, or a sub-definition thereof, X, and any group associated therewith, is as defined in paragraph (11) above and R₁ is as defined in paragraph (18) above.

Suitably, in any of the definitions of formulae I or II set out herein, R₂ is as defined in any one of paragraphs (19) to (29) above. More suitably, R₂ is as defined in any one of paragraphs (21) to (29) above. Even more suitably, R₂ is as defined in any one of paragraphs (23) to (29) above. Yet more suitably, R₂ is as defined in any one of paragraphs (25) to (29) above. Yet even more suitably, R₂ is as defined in any one of paragraphs (26), (27), (28) or (29) above. Most suitably, R₂ is as defined in any one of paragraphs (28) or (29) above.

In a particular group of compounds of formulae I or II wherein R is -(1-4C)alkylene-R₂, or a sub-definition thereof, R₂, and any group associated therewith, is as defined in paragraph (19) above.

In a particular group of compounds of formulae I or II wherein R is -(1-4C)alkylene-R₂, or a sub-definition thereof, R₂, and any group associated therewith, is as defined in paragraph (20) above.

In a particular group of compounds of formulae I or II wherein R is -(1-4C)alkylene-R₂, or a sub-definition thereof, R₂, and any group associated therewith, is as defined in paragraph (21) above.

In a particular group of compounds of formulae I or II wherein R is -(1-4C)alkylene-R₂, or a sub-definition thereof, R₂, and any group associated therewith, is as defined in paragraph (22) above.

In a particular group of compounds of formulae I or II wherein R is -(1-4C)alkylene-R₂, or a sub-definition thereof, R₂, and any group associated therewith, is as defined in paragraph (23) above.

In a particular group of compounds of formulae I or II wherein R is -(1-4C)alkylene-R₂, or a sub-definition thereof, R₂, and any group associated therewith, is as defined in paragraph (24) above.

In a particular group of compounds of formulae I or II wherein R is -(1-4C)alkylene-R₂, or a sub-definition thereof, R₂, and any group associated therewith, is as defined in paragraph (25) above.

In a particular group of compounds of formulae I or II wherein R is -(1-4C)alkylene-R₂, or a sub-definition thereof, R₂, and any group associated therewith, is as defined in paragraph (26) above.

In a particular group of compounds of formulae I or II wherein R is -(1-4C)alkylene-R₂, or a sub-definition thereof, R₂, and any group associated therewith, is as defined in paragraph (27) above.

In a particular group of compounds of formulae I or II wherein R is -(1-4C)alkylene-R₂, or a sub-definition thereof, R₂, and any group associated therewith, is as defined in paragraph (28) above.

In a particular group of compounds of formulae I or II wherein R is -(1-4C)alkylene-R₂, or a sub-definition thereof, R₂, and any group associated therewith, is as defined in paragraph (29) above.

In a particular group of compounds of the invention, the compound is a compound of formula II defined herein in which R is -methylene-X—R₁ (as defined in paragraph (5) above), i.e. the compounds have the formula Ila shown below, or a pharmaceutically acceptable salt thereof:

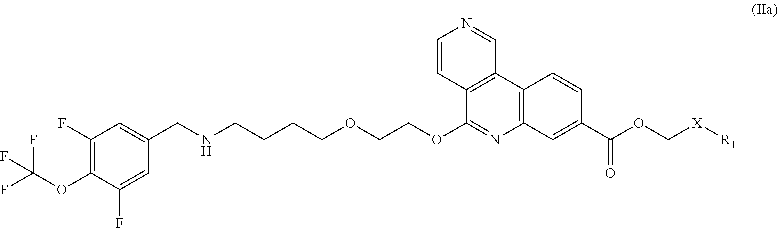

wherein X and R₁ have any one of the definitions set out herein.

In a particular group of compounds of formula Ila, X is as defined in any one of paragraphs (6) to (11) above and R₁ is as defined in any one of paragraphs (12) to (18) above. Suitably, X is as defined in any one of paragraphs (7) to (11) above and R₁ is as defined in any one of paragraphs (13) to (18) above. More suitably, X is as defined in any one of paragraphs (8) to (11) above and R₁ is as defined in any one of paragraphs (14) to (18) above. Yet more suitably, X is as defined in any one of paragraphs (9), (10) or (11) above and R₁ is as defined in any one of paragraphs (15) to (18) above. Even more suitably, X is as defined in any one of paragraphs (10) or (11) above and R₁ is as defined in any one of paragraphs (16), (17) or (18) above. Yet even more suitably, X is as defined in paragraph (11) above and R₁ is as defined in any one of paragraphs (17) or (18) above. Most suitably, X is as defined in paragraph (11) above and R₁ is as defined in paragraph (18) above.

In a particular group of compounds of the invention, the compound is a compound of formula II defined herein in which R is -methylene-X—R₁ (as defined in paragraph (5) above) and X is —O—C(O)— (as defined in paragraph (11) above), i.e. the compounds have the formula IIb shown below, or a pharmaceutically acceptable salt thereof:

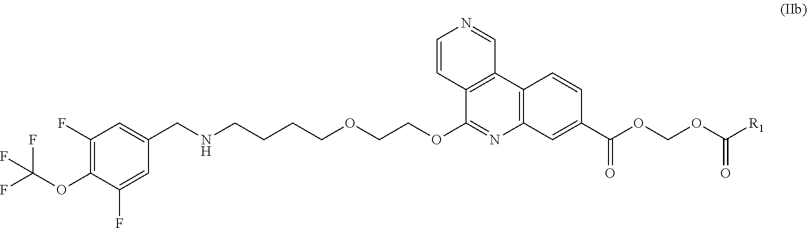

wherein R₁ has any one of the definitions set out herein.

In a particular group of compounds of formula IIb, R₁ is as defined in any one of paragraphs (12) to (18) above. Suitably, R₁ is as defined in any one of paragraphs (13) to (18) above. More suitably, R₁ is as defined in any one of paragraphs (14) to (18) above. Yet more suitably, R₁ is as defined in any one of paragraphs (15) to (18) above. Even more suitably, R₁ is as defined in any one of paragraphs (16), (17) or (18) above. Yet even more suitably, R₁ is as defined in any one of paragraphs (17) or (18) above. Most suitably, R₁ is as defined in paragraph (18) above.

In a particular group of compounds of the invention, the compound is a compound of formula II defined herein in which R is -methylene-X—R₁ (as defined in paragraph (5) above) and X is —O—C(O)—O— (as defined in paragraph (11) above), i.e. the compounds have the formula IIc shown below, or a pharmaceutically acceptable salt thereof:

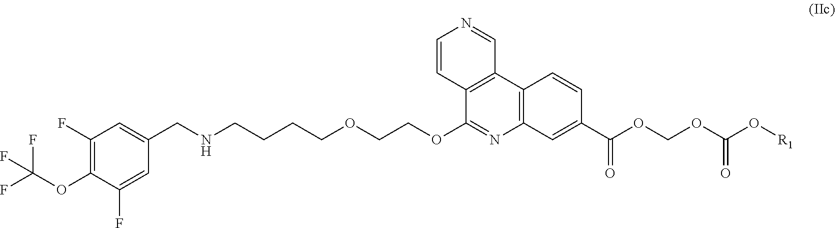

wherein R₁ has any one of the definitions set out herein.

In a particular group of compounds of formula IIc, R₁ is as defined in any one of paragraphs (12) to (18) above. Suitably, R₁ is as defined in any one of paragraphs (13) to (18) above. More suitably, R₁ is as defined in any one of paragraphs (14) to (18) above. Yet more suitably, R₁ is as defined in any one of paragraphs (15) to (18) above. Even more suitably, R₁ is as defined in any one of paragraphs (16), (17) or (18) above. Yet even more suitably, R₁ is as defined in any one of paragraphs (17) or (18) above. Most suitably, R₁ is as defined in paragraph (18) above.

In a particular group of compounds of the invention, the compound is a compound of formula II defined herein in which R is -ethylene-X—R₁ (as defined in paragraph (5) above), i.e. the compounds have the formula lid shown below, or a pharmaceutically acceptable salt thereof:

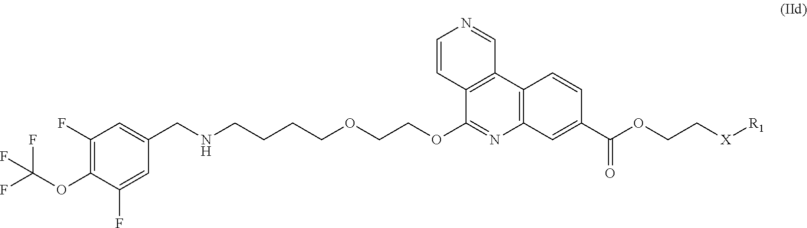

wherein X and R₁ have any one of the definitions set out herein.

In a particular group of compounds of formula lid, X is as defined in any one of paragraphs (6) to (11) above and R₁ is as defined in any one of paragraphs (12) to (18) above. Suitably, X is as defined in any one of paragraphs (7) to (11) above and R₁ is as defined in any one of paragraphs (13) to (18) above. More suitably, X is as defined in any one of paragraphs (8) to (11) above and R₁ is as defined in any one of paragraphs (14) to (18) above. Yet more suitably, X is as defined in any one of paragraphs (9), (10) or (11) above and R₁ is as defined in any one of paragraphs (15) to (18) above. Even more suitably, X is as defined in any one of paragraphs (10) or (11) above and R₁ is as defined in any one of paragraphs (16), (17) or (18) above. Yet even more suitably, X is as defined in paragraph (11) above and R₁ is as defined in any one of paragraphs (17) or (18) above. Most suitably, X is as defined in paragraph (11) above and R₁ is as defined in paragraph (18) above.

In a particular group of compounds of the invention, the compound is a compound of formula II defined herein in which R is -ethylene-X—R₁ (as defined in paragraph (5) above), X is —N(Me)— (as defined in paragraph (11) above), i.e. the compounds have the formula lie shown below, or a pharmaceutically acceptable salt thereof:

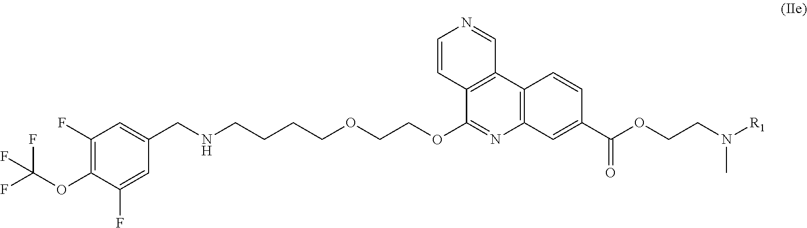

wherein R₁ has any one of the definitions set out herein.

In a particular group of compounds of formula lie, R₁ is as defined in any one of paragraphs (12) to (18) above. Suitably, R₁ is as defined in any one of paragraphs (13) to (18) above. More suitably, R₁ is as defined in any one of paragraphs (14) to (18) above. Yet more suitably, R₁ is as defined in any one of paragraphs (15) to (18) above. Even more suitably, R₁ is as defined in any one of paragraphs (16), (17) or (18) above. Yet even more suitably, R₁ is as defined in any one of paragraphs (17) or (18) above. Most suitably, R₁ is as defined in paragraph (18) above.

In a particular group of compounds of the invention, the compound is a compound of formula II defined herein in which R is -methylene-R₂ (as defined in paragraph (5) above), i.e. the compounds have the formula IIf shown below, or a pharmaceutically acceptable salt thereof:

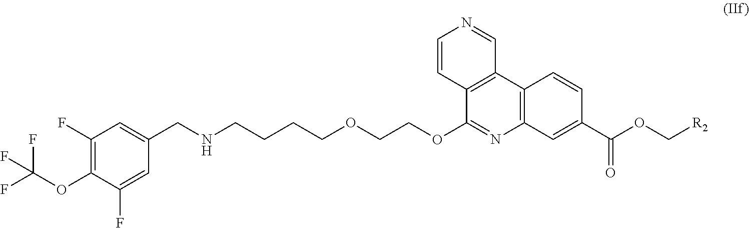

wherein R₂ has any one of the definitions set out herein.

In a particular group of compounds of formula IIf, R₂ is as defined in any one of paragraphs (19) to (29) above. Suitably, R₂ is as defined in any one of paragraphs (21) to (29) above. More suitably, R₂ is as defined in any one of paragraphs (23) to (29) above. Even more suitably, R₂ is as defined in any one of paragraphs (25) to (29) above. Yet more suitably, R₂ is as defined in any one of paragraphs (26), (27), (28) or (29) above. Yet even more suitably, R₂ is as defined in any one of paragraphs (27), (28) or (29) above. Yet still even more suitably, R₂ is as defined in any one of paragraphs (28) or (29) above. Most suitably, R₂ is as defined in paragraph (29) above.

Particular compounds of the present invention include any of the compounds described in the example section of the present application, or a pharmaceutically acceptable salt, hydrate or solvate thereof, and, in particular, any of the following:
isopropyl 5-(2-(4-((3,5-difluoro-4-(trifluoromethoxy)benzyl)amino)butoxy)ethoxy)benzo[c][2,6]naphthyridine-8-carboxylate;
2-(dimethylamino)ethyl 5-(2-(4-((3,5-difluoro-4-(trifluoromethoxy)benzyl)amino)butoxy)ethoxy)benzo[c][2,6]naphthyridine-8-carboxylate;
methyl 5-(2-(4-((3,5-difluoro-4-(trifluoromethoxy)benzyl)amino)butoxy)ethoxy)benzo[c][2,6]naphthyridine-8-carboxylate;
(isobutyryloxy)methyl 5-(2-(4-((3,5-difluoro-4-(trifluoromethoxy)benzyl)amino)butoxy)ethoxy)benzo[c][2,6]naphthyridine-8-carboxylate;
(pivaloyloxy)methyl 5-(2-(4-((3,5-difluoro-4-(trifluoromethoxy)benzyl)amino)butoxy)ethoxy)benzo[c][2,6]naphthyridine-8-carboxylate;
(5-methyl-2-oxo-1,3-dioxol-4-yl)methyl 5-(2-(4-((3,5-difluoro-4-(trifluoromethoxy)benzyl)amino)butoxy)ethoxy)benzo[c][2,6]naphthyridine-8-carboxylate;
ethyl 5-(2-(4-((3,5-difluoro-4-(trifluoromethoxy)benzyl)amino)butoxy)ethoxy)benzo[c][2,6]naphthyridine-8-carboxylate;
tert-butyl 5-(2-(4-((3,5-difluoro-4-(trifluoromethoxy)benzyl)amino)butoxy)ethoxy)benzo[c][2,6]naphthyridine-8-carboxylate;
acetoxymethyl 5-(2-(4-((3,5-difluoro-4-(trifluoromethoxy)benzyl)amino)butoxy)ethoxy)benzo[c][2,6]naphthyridine-8-carboxylate;
((methoxycarbonyl)oxy)methyl 5-(2-(4-((3,5-difluoro-4-(trifluoromethoxy)benzyl)amino)butoxy)ethoxy)benzo[c][2,6]naphthyridine-8-carboxylate;
(propionyloxy)methyl 5-(2-(4-((3,5-difluoro-4-(trifluoromethoxy)benzyl)amino)butoxy)ethoxy)benzo[c][2,6]naphthyridine-8-carboxylate;
((ethoxycarbonyl)oxy)methyl 5-(2-(4-((3,5-difluoro-4-(trifluoromethoxy)benzyl)amino)butoxy)ethoxy)benzo[c][2,6]naphthyridine-8-carboxylate;
((isopropoxycarbonyl)oxy)methyl 5-(2-(4-((3,5-difluoro-4-(trifluoromethoxy)benzyl)amino)butoxy)ethoxy)benzo[c][2,6]naphthyridine-8-carboxylate;
((tert-butoxycarbonyl)oxy)methyl 5-(2-(4-((3,5-difluoro-4-(trifluoromethoxy)benzyl)amino)butoxy)ethoxy)benzo[c][2,6]naphthyridine-8-carboxylate; and
((((tetrahydro-2H-pyran-4-yl)oxy)carbonyl)oxy)methyl 5-(2-(4-((3,5-difluoro-4-(trifluoromethoxy)benzyl)amino)butoxy)ethoxy)benzo[c][2,6]naphthyridine-8-carboxylate.

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Salts and Solvates

The compounds (including final products and intermediates) described herein may be isolated and used per se or may be isolated in the form of a salt, suitably pharmaceutically acceptable salts. It should be understood that the terms “salt(s)” and “salt form(s)” used by themselves or in conjunction with another term or terms encompasses all inorganic and organic salts, including industrially acceptable salts, as defined herein, and pharmaceutically acceptable salts, as defined herein, unless otherwise specified. As used herein, industrially acceptable salts are salts that are generally suitable for manufacturing and/or processing (including purification) as well as for shipping and storage, but may not be salts that are typically administered for clinical or therapeutic use. Industrially acceptable salts may be prepared on a laboratory scale, i.e. multi-gram or smaller, or on a larger scale, i.e. up to and including a kilogram or more.

Pharmaceutically acceptable salts, as used herein, are salts that are generally chemically and/or physically compatible with the other ingredients comprising a formulation, and/or are generally physiologically compatible with the recipient thereof. Pharmaceutically acceptable salts may be prepared on a laboratory scale, i.e. multi-gram or smaller, or on a larger scale, i.e. up to and including a kilogram or more. It should be understood that pharmaceutically acceptable salts are not limited to salts that are typically administered or approved by the FDA or equivalent foreign regulatory body for clinical or therapeutic use in humans. A practitioner of ordinary skill will readily appreciate that some salts are both industrially acceptable as well as pharmaceutically acceptable salts. It should be understood that all such salts, including mixed salt forms, are within the scope of the application.

In one embodiment, the compounds of Formulae I and II and sub-formulae thereof are isolated as pharmaceutically acceptable salts.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

In general, salts of the present application can be prepared in situ during the isolation and/or purification of a compound (including intermediates), or by separately reacting the compound (or intermediate) with a suitable organic or inorganic acid or base (as appropriate) and isolating the salt thus formed. The degree of ionisation in the salt may vary from completely ionised to almost non-ionised. In practice, the various salts may be precipitated (with or without the addition of one or more co-solvents and/or anti-solvents) and collected by filtration or the salts may be recovered by evaporation of solvent(s). Salts of the present application may also be formed via a “salt switch” or ion exchange/double displacement reaction, i.e. reaction in which one ion is replaced (wholly or in part) with another ion having the same charge. One skilled in the art will appreciate that the salts may be prepared and/or isolated using a single method or a combination of methods.

Representative salts include, but are not limited to, acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate and the like. Other examples of representative salts include alkali or alkaline earth metal cations such as, but not limited to, sodium, lithium, potassium, calcium, magnesium, and the like, as well as non-toxic ammonium, quaternary ammonium and amine cations including, but not limited to, ammonium, tetramethylammonium, tetraethylammonium, lysine, arginine, benzathine, choline, tromethamine, diolamine, glycine, meglumine, olamine and the like.

Certain compounds of the Formulae I and II and sub-formulae thereof may exist in solvated as well as unsolvated forms such as, for example, hydrated forms. It is to be understood that the invention encompasses all such solvated forms that possess the biological activity described herein.

### Polymorphs

It is also to be understood that certain compounds of the Formulae I and II and sub-formulae thereof may exhibit polymorphism, and that the invention encompasses all such forms that possess the biological activity described herein.

### N-Oxides

Compounds of the Formulae I and II and sub-formulae thereof containing an amine function may also form N-oxides. A reference herein to a compound of the Formulae I and II and sub-formulae thereof that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as, but not limited to, hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as, but not limited to, dichloromethane.

### Tautomers

Compounds of the Formulae I and II and sub-formulae thereof may exist in a number of different tautomeric forms and references to compounds of the Formulae I and II and sub-formulae thereof include all such forms. For the avoidance of doubt, where a compound can exist in one of several tautomeric forms, and only one is specifically described or shown, all others are nevertheless embraced by Formulae I and II and sub-formulae thereof. Examples of tautomeric forms include keto-, enol-, and enolate-forms, as in, for example, the following tautomeric pairs: keto/enol (illustrated below), pyrimidone/hydroxypyrimidine, imine/enamine, amide/imino alcohol, amidine/amidine, nitroso/oxime, thioketone/enethiol, and nitro/aci-nitro.

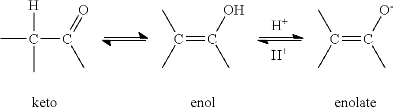

### Isomers

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterized by the absolute configuration of its asymmetric centre and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

Certain compounds of Formulae I and II and sub-formulae thereof may have one or more asymmetric centres and therefore can exist in a number of stereoisomeric configurations. Consequently, such compounds can be synthesized and/or isolated as mixtures of enantiomers and/or as individual (pure) enantiomers, and, in the case of two or more asymmetric centres, single diastereomers and/or mixtures of diastereomers. It should be understood that the present application includes all such enantiomers and diastereomers and mixtures thereof in all ratios.

### Isotopes

The compounds of the present invention are described herein using structural formulas that do not specifically recite the mass numbers or the isotope ratios of the constituent atoms. As such it is intended that the present application includes compounds in which the constituent atoms are present in any ratio of isotope forms. For example, carbon atoms may be present in any ratio of ¹²C, ¹³C, and ¹⁴C; hydrogen atoms may be present in any ratio of ¹H, ²H, and ³H; etc. Preferably, the constituent atoms in the compounds of the present invention are present in their naturally occurring ratios of isotope forms.

### Pharmaceutical Compositions

According to a further aspect of the invention there is provided a pharmaceutical composition which comprises a compound of the invention as defined herein, or a pharmaceutically acceptable salt, hydrate or solvate thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 1.5 g of active agent (more suitably from 0.5 to 600 mg, for example from 1 to 200 mg) compounded with an appropriate and convenient amount of excipients which may vary from about 5 to about 98 percent by weight of the total composition.

The size of the dose for therapeutic or prophylactic purposes of a compound of the Formulae I and II will naturally vary according to the nature and severity of the conditions, the age and sex of the animal or patient and the route of administration, according to well-known principles of medicine.

It is to be noted that dosages and dosing regimens may vary with the type and severity of the condition to be alleviated, and may include the administration of single or multiple doses, i.e. QD (once daily), BID (twice daily), etc., over a particular period of time (days or hours). It is to be further understood that for any particular subject or patient, specific dosage regimens may need to be adjusted over time according to the individual need and the professional judgment of the person administering or supervising the administration of the pharmaceutical compositions. For example, doses may be adjusted based on pharmacokinetic or pharmacodynamic parameters, which may include clinical effects such as toxic effects and/or laboratory values. Thus, the present application encompasses intra-patient dose-escalation as determined by the person skilled in the art. Procedures and processes for determining the appropriate dosage(s) and dosing regimen(s) are well-known in the relevant art and would readily be ascertained by the skilled artisan. As such, one of ordinary skill would readily appreciate and recognize that the dosage ranges set forth herein are exemplary only and are not intended to limit the scope or practice of the pharmaceutical compositions described herein.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used.

For the compounds of the present invention, oral administration is particularly suitable. The compounds of the present invention may be formulated as a tablet, capsule or solution for oral administration. Suitably, the compound of the present invention is formulated in a unit dosage form (e.g. a tablet or capsule) for oral administration. Typically, unit dosage forms will contain about 0.5 mg to 1.5 g of a compound of this invention.

### Therapeutic Uses and Applications

The compounds of the present invention are potent inhibitors of Casein Kinase 2 alpha (CK2α). Data showing the CK2α inhibition for the exemplified compounds is presented in the accompanying example section.

The compounds of the present invention are designed to bind to the catalytic ATP site of CK2α (to drive potent enzyme inhibition) and the aD site (to drive high levels of selectivity over other kinases) [Brear et al, Chem Sci 2016].

Accordingly, the compounds of formulae I and II are useful for the treatment and/or prevention of diseases and conditions in which CK2α activity is implicated, such as, for example, but not limited to, the treatment and/or prevention of proliferative disorders (e.g. cancer), viral infections, inflammation, diabetes, vascular and ischemic disorders, neurodegeneration and the regulation of circadian rhythm.

In another aspect, the present invention provides a compound of Formulae I or II as defined herein, or a pharmaceutically acceptable salt, hydrate or solvate thereof, or a pharmaceutical composition as defined herein, for use in therapy.

In another aspect, the present invention provides a compound of Formulae I or II as defined herein, or a pharmaceutically acceptable salt, hydrate or solvate thereof, or a pharmaceutical composition as defined herein, for use in the treatment of a disease or condition in which CK2α activity is implicated.

In another aspect, the present invention provides the use of a compound of Formulae I or II as defined herein, or a pharmaceutically acceptable salt, hydrate or solvate thereof, in the manufacture of a medicament for use in the treatment of a disease or condition in which CK2α activity is implicated.

In another aspect, the present invention provides a method of treating a disease or condition in which CK2α activity is implicated, said method comprising administering to a subject in need thereof an effective amount of a compound of Formulae I or II as defined herein, or a pharmaceutically acceptable salt, hydrate or solvate thereof, or a pharmaceutical composition as defined herein.

In another aspect, the present invention provides a compound of Formulae I or II as defined herein, or a pharmaceutically acceptable salt, hydrate or solvate thereof, or a pharmaceutical composition as defined herein, for use in the treatment of a disease or condition associated with aberrant activity of CK2α.

In another aspect, the present invention provides the use of a compound of Formulae I or II as defined herein, or a pharmaceutically acceptable salt, hydrate or solvate thereof, in the manufacture of a medicament for use in the treatment of a disease or condition associated with aberrant activity of CK2α.

In another aspect, the present invention provides a method of treating a disease or condition associated with aberrant activity of CK2α, said method comprising administering to a subject in need thereof an effective amount of a compound of Formulae I or II as defined herein, or a pharmaceutically acceptable salt, hydrate or solvate thereof, or a pharmaceutical composition as defined herein.

In another aspect, the present invention provides a compound of Formulae I or II as defined herein, or a pharmaceutically acceptable salt, hydrate or solvate thereof, or a pharmaceutical composition as defined herein, for use in the treatment of proliferative disorders (e.g. cancer or benign neoplasms), viral infections, an inflammatory disease or condition, diabetes, vascular and ischemic disorders, neurodegenerative disorders and/or the regulation of circadian rhythm.

In another aspect, the present invention provides the use of a compound of Formulae I or II as defined herein, or a pharmaceutically acceptable salt, hydrate or solvate thereof, in the manufacture of a medicament for use in the treatment of proliferative disorders (e.g. cancer or benign neoplasms), viral infections, an inflammatory disease or condition, diabetes, vascular and ischemic disorders, neurodegenerative disorders and/or the regulation of circadian rhythm.

In another aspect, the present invention provides a method of treating a proliferative disorder (e.g. cancer or benign neoplasms), a viral infection, an inflammatory disease or condition, diabetes, vascular and ischemic disorders, neurodegenerative disorders and/or regulating cardiac rhythm, said method comprising administering to a subject in need thereof an effective amount of a compound of Formulae I or II as defined herein, or a pharmaceutically acceptable salt, hydrate or solvate thereof, or a pharmaceutical composition as defined herein.

In another aspect, the present invention provides a compound of Formulae I or II as defined herein, or a pharmaceutically acceptable salt, hydrate or solvate thereof, or a pharmaceutical composition as defined herein, for use in the treatment of a proliferative disorder.

In another aspect, the present invention provides the use of a compound of Formulae I or II as defined herein, or a pharmaceutically acceptable salt, hydrate or solvate thereof, in the manufacture of a medicament for use in the treatment of a proliferative disorder (e.g. cancer or a benign neoplasms).

In another aspect, the present invention provides a method of treating a proliferative disorder (e.g. cancer or benign neoplasms), said method comprising administering to a subject in need thereof an effective amount of a compound of Formulae I or II as defined herein, or a pharmaceutically acceptable salt, hydrate or solvate thereof, or a pharmaceutical composition as defined herein.

The terms “proliferative disorder” and “proliferative condition” are used interchangeably herein and pertain to an unwanted or uncontrolled cellular proliferation of excessive or abnormal cells which is undesired, such as, neoplastic or hyperplastic growth, whether in vitro or in vivo.

Examples of proliferative conditions include, but are not limited to, pre-malignant and malignant cellular proliferation, including but not limited to, cancers, psoriasis, bone diseases, fibroproliferative disorders (e.g. of connective tissues), and atherosclerosis.

Any type of cell may be treated, including but not limited to, lung, colon, breast, ovarian, prostate, liver, pancreas, brain, blood and skin.

In certain aspects of the present invention, the proliferative disorder is cancer, suitably a cancer selected from lung, colon/colorectal, breast, ovarian, prostate, liver, pancreas, brain, blood, cholangiocarcinoma and skin cancer.

In a particular aspect of the invention, the proliferative disorder is colon/colorectal, cholangiocarcinoma, ovarian or prostate cancer.

In a particular aspect of the invention, the proliferative disorder is colorectal cancer.

In certain aspects of the present invention, the proliferative disorder is hematopoietic tumour, including: myelogenous and granulocytic leukemia (malignancy of the myeloid and granulocytic white blood cell series); lymphatic, lymphocytic, and lymphoblastic leukemia (malignancy of the lymphoid and lymphocytic blood cell series); polycythemia vera and erythremia (malignancy of various blood cell products, but with red cells predominating); and myelofibrosis.

A benign neoplasm may be, for example, hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas, pyogenic granulomas, moles, uterine fibroids, thyroid adenomas, adrenocortical adenomas or pituitary adenomas. The benign neoplasm may be endometrial implants or a keratocystic odontogenic tumor.

In another aspect, the present invention provides a compound of Formulae I or II as defined herein, or a pharmaceutically acceptable salt, hydrate or solvate thereof, or a pharmaceutical composition as defined herein, for use in the treatment of a cancer.

In another aspect, the present invention the use of a compound of Formulae I or II as defined herein, or a pharmaceutically acceptable salt, hydrate or solvate thereof, in the manufacture of a medicament for use in the treatment of a cancer.

In another aspect, the present invention provides a method of treating cancer, said method comprising administering to a subject in need thereof an effective amount of a compound of Formulae I or II as defined herein, or a pharmaceutically acceptable salt, hydrate or solvate thereof, or a pharmaceutical composition as defined herein.

The cancer may be non-metastatic or metastatic and which may be a solid tumour or a haematological (“liquid”) cancer. The cancer may, for example, be selected from:
(1) Carcinoma, including for example tumours derived from stratified squamous epithelia (squamous cell carcinomas) and tumours arising within organs or glands (adenocarcinomas). Examples include breast, colon, lung, prostate, ovary, esophageal carcinoma (including, but not limited to, esophageal adenocarcinoma and squamous cell carcinoma), basal-like breast carcinoma, basal cell carcinoma (a form of skin cancer), squamous cell carcinoma (various tissues), head and neck carcinoma (including, but not limited to, squamous cell carcinomas), stomach carcinoma (including, but not limited to, stomach adenocarcinoma, gastrointestinal stromal tumor), signet ring cell carcinoma, bladder carcinoma (including transitional cell carcinoma (a malignant neoplasm of the bladder)), bronchogenic carcinoma, colorectal carcinoma (including, but not limited to, colon carcinoma and rectal carcinoma), anal carcinoma, gastric carcinoma, lung carcinoma (including but not limited to small cell carcinoma (SCLC) and non-small cell carcinoma of the lung (NSCLC), lung adenocarcinoma, squamous cell carcinoma, large cell carcinoma, bronchioloalveolar carcinoma, and mesothelioma), neuroendocrine tumors (including but not limited to carcinoids of the gastrointestinal tract, breast, and other organs), adrenocortical carcinoma, thyroid carcinoma, pancreatic carcinoma (including, but not limited to, pancreatic ductal adenocarcinoma, pancreatic adenocarcinoma, acinar cell carcinoma, intraductal papillary mucinous neoplasm with invasive carcinoma, mucinous cystic neoplasm with invasive carcinoma, islet cell carcinoma and neuroendocrine tumors), breast carcinoma (including, but not limited to, ductal carcinoma, lobular carcinoma, inflammatory breast cancer, clear cell carcinoma, mucinous carcinoma), ovarian carcinoma (including, but not limited to, ovarian epithelial carcinoma or surface epithelial-stromal tumor including serous tumor, endometrioid tumor and mucinous cystadenocarcinoma, sex-cord-stromal tumor), liver and bile duct carcinoma (including, but not limited to, hepatocellular carcinoma, cholangiocarcinoma and hemangioma), prostate carcinoma, adenocarcinoma, brain tumours (including, but not limited to glioma, glioblastoma and medulloblastoma), germ cell tumors, sweat gland carcinoma, sebaceous gland carcinoma, papillary carcinoma, papillary adenocarcinoma, cystadenocarcinoma, kidney carcinoma (including, but not limited to, renal cell carcinoma, clear cell carcinoma and Wilm's tumor), medullary carcinoma, ductal carcinoma in situ or bile duct carcinoma, choriocarcinoma, seminoma, embryonal carcinoma, cervical carcinoma, uterine carcinoma (including, but not limited to, endometrial adenocarcinoma, uterine papillary serous carcinoma, uterine clear-cell carcinoma, uterine sarcomas and leiomyosarcomas, mixed mullerian tumors), testicular carcinoma, osteogenic carcinoma, epithelial carcinoma, sarcomatoid carcinoma, nasopharyngeal carcinoma, laryngeal carcinoma; oral and oropharyngeal squamous carcinoma;
(2) Sarcomas, including: osteosarcoma and osteogenic sarcoma (bone); chondrosarcoma (cartilage); leiomyosarcoma (smooth muscle); rhabdomyosarcoma (skeletal muscle); mesothelial sarcoma and mesothelioma (membranous lining of body cavities); fibrosarcoma (fibrous tissue); angiosarcoma and hemangioendothelioma (blood vessels); liposarcoma (adipose tissue); glioma and astrocytoma (neurogenic connective tissue found in the brain); myxosarcoma (primitive embryonic connective tissue); chordoma, endotheliosarcoma, lymphangiosarcoma, lymphangioendotheliosarcoma, synovioma, Ewing's sarcoma, mesenchymous and mixed mesodermal tumor (mixed connective tissue types) and other soft tissue sarcomas;
(3) Myeloma and multiple myeloma;
(4) Hematopoietic tumours, including: myelogenous and granulocytic leukemia (malignancy of the myeloid and granulocytic white blood cell series); lymphatic, lymphocytic, and lymphoblastic leukemia (malignancy of the lymphoid and lymphocytic blood cell series); polycythemia vera and erythremia (malignancy of various blood cell products, but with red cells predominating); myelofibrosis;
(5) Lymphomas, including: Hodgkin and Non-Hodgkin lymphomas;
(6) Solid tumors of the nervous system including medulloblastoma, craniopharyngioma, ependymoma, pinealoma, hemangioblastoma, acoustic neuroma, oligodendroglioma, meningioma, neuroblastoma and schwannoma;
(7) Melanoma, uveal melanoma and retinoblastoma; and
(8) Mixed Types, including, e.g., adenosquamous carcinoma, mixed mesodermal tumor, carcinosarcoma or teratocarcinoma.

Suitably, a compound of the invention, or a pharmaceutically acceptable salt thereof may be for use in the treatment of a cancer selected from cancer selected from colon/colorectal cancer, cholangiocarcinoma, gastric cancer, skin cancer (e.g. basal cell carcinoma), ovarian, prostate, breast cancer, liver cancer, pancreatic cancer, brain cancer, blood cancers (leukaemia's, myelomas), bladder cancer, bone cancer, head and neck cancer, renal cancer and lung cancer.

More suitably, the cancer is selected from colon/colorectal cancer, prostate cancer, ovarian cancer, basal cell carcinoma or cholangiocarcinoma.

In a particular aspect of the present invention, the cancer is basal cell carcinoma.

In a particular aspect of the present invention, the cancer is colorectal cancer.

In a particular aspect of the present invention, the cancer is cholangiocarcinoma.

In a further aspect of the present invention, the cancer is prostate cancer.

In a further aspect of the present invention, the cancer is ovarian cancer.

In another aspect of the present invention, the cancer is a hematopoietic tumour.

It is hypothesised that the compounds of the present invention will be particularly suited to the treatment of wnt pathway driven cancers, e.g. wnt pathway mutated colorectal cancer or cholangiocarcinoma (Di Maira et al, 2019).

In addition to CK2α having a very well characterized function in wnt pathway activity, it also plays a role in other key cellular pathways known to be upregulated in cancer, such as, but not limited to, the DNA damage response (Ruzzene & Pinna, 2010; Montenarh, Transl. Cancer Res 2016). Thus, the compounds of the present invention may have a further use in the treatment of PARP insensitive tumors in prostate/ovarian cancer.

CK2α has also recently been identified as a key host protein required for viral replication (e.g. in SARS-CoV2) and as such could represent an antiviral treatment (Gordon et al. Nature 2020).

Thus, in another aspect, the present invention provides a compound of Formulae I or II as defined herein, or a pharmaceutically acceptable salt, hydrate or solvate thereof, or a pharmaceutical composition as defined herein, for use in the treatment of a viral infection.

In another aspect, the present invention provides the use of a compound of Formulae I or II as defined herein, or a pharmaceutically acceptable salt, hydrate or solvate thereof, in the manufacture of a medicament for use in the treatment of a viral infection.

In another aspect, the present invention provides a method of treating a viral infection, said method comprising administering to a subject in need thereof an effective amount of a compound of Formulae I or II as defined herein, or a pharmaceutically acceptable salt, hydrate or solvate thereof, or a pharmaceutical composition as defined herein.

Suitably, the virus is a coronavirus, e.g. SARS-CoV2.

### Routes of Administration

The compounds of the invention or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (e.g. by a patch, plaster, etc.); transmucosal (e.g. by a patch, plaster, etc.); intranasal (e.g. by nasal spray); ocular (e.g. by eye drops, eye ointment etc.); pulmonary (e.g. by inhalation or insufflation therapy, for example via an aerosol, for example by the nose or mouth); rectal (e.g. by suppository or enema); vaginal (e.g. by pessary); parental, for example by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir dosage form, for example subcutaneously or intramuscularly.

The compounds of the present invention are particularly suitable for oral administration.

### Combination Therapies

The compounds of the invention and salts, solvates thereof defined herein may be applied as a sole therapy or may involve, in addition to the compound of the invention, one or more additional therapeutic agents, e.g. an anti-tumour agent.

In the context of cancer treatment, in addition to the compound of the invention therapy may involve conventional surgery or radiotherapy or chemotherapy. Such chemotherapy may include one or more of the following categories of anti-tumour agents:
other antiproliferative/antineoplastic drugs and combinations thereof, as used in medical oncology, such as, but not limited to, alkylating agents (for example cisplatin, oxaliplatin, carboplatin, cyclophosphamide, nitrogen mustard, melphalan, chlorambucil, busulphan, temozolamide and nitrosoureas); antimetabolites (for example gemcitabine and antifolates such as, but not limited to, fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
cytostatic agents such as, but not limited to, antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as, but not limited to, finasteride;
anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase];
inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. (Critical reviews in oncology/haematology, 2005, Vol. 54, pp11-29); such inhibitors also include tyrosine kinase inhibitors, for example inhibitors of the epidermal growth factor family (for example EGFR family tyrosine kinase inhibitors such as, but not limited to, N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as, but not limited to, lapatinib); inhibitors of the hepatocyte growth factor family; inhibitors of the insulin growth factor family; inhibitors of the platelet-derived growth factor family such as, but not limited to, imatinib and/or nilotinib (AMN107); inhibitors of serine/threonine kinases (for example Ras/Raf signalling inhibitors such as, but not limited to, farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as, but not limited to, CDK2 and/or CDK4 inhibitors;
antiangiogenic agents such as, but not limited to, those which inhibit the effects of vascular endothelial growth factor, [for example the anti-vascular endothelial cell growth factor antibody bevacizumab (Avastin™) and for example, a VEGF receptor tyrosine kinase inhibitor such as, but not limited to, vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as, but not limited to, those disclosed in International Patent Applications WO97/22596, WO 97/30035, WO 97/32856 and WO 98/13354 and compounds that work by other mechanisms (for example linomide, inhibitors of integrin αvβ3 function and angiostatin)];
vascular damaging agents such as, but not limited to, Combretastatin A4 and compounds disclosed in International Patent Applications WO 99/02166, WO 00/40529, WO 00/41669, WO 01/92224, WO 02/04434 and WO 02/08213;
an endothelin receptor antagonist, for example zibotentan (ZD4054) or atrasentan;
antisense therapies, for example those which are directed to the targets listed above, such as, but not limited to, ISIS 2503, an anti-ras antisense;
gene therapy approaches, including for example approaches to replace aberrant genes such as, but not limited to, aberrant p53 or aberrant BRCA1 or BRCA2, GDEPT (gene-directed enzyme pro-drug therapy) approaches such as, but not limited to, those using cytosine deaminase, thymidine kinase or a bacterial nitroreductase enzyme and approaches to increase patient tolerance to chemotherapy or radiotherapy such as multi-drug resistance gene therapy; and
immunotherapy approaches, including for example ex-vivo and in-vivo approaches to increase the immunogenicity of patient tumour cells, such as, but not limited to, transfection with cytokines such as interleukin 2, interleukin 4 or granulocyte-macrophage colony stimulating factor, approaches to decrease T-cell anergy, approaches using transfected immune cells such as, but not limited to, cytokine-transfected dendritic cells, approaches using cytokine-transfected tumour cell lines and approaches using anti-idiotypic antibodies.

In a particular embodiment, the antiproliferative treatment defined herein may involve, in addition to the compound of the invention, conventional surgery or radiotherapy or chemotherapy.

In a further particular embodiment, the antiproliferative treatment defined herein may involve, in addition to the compound of the invention, standard chemotherapy for the cancer concerned.

In a particular embodiment, the antiproliferative treatment defined herein may involve, in addition to the compound of the invention, therapy with K-ras inhibitors and/or DNA damage repair inhibitors (e.g. PARP inhibitors).

Such conjoint treatment may be achieved by way of the simultaneous, sequential or separate dosing of the individual components of the treatment. Such combination products employ the compounds of this invention within the dosage range described herein and the other pharmaceutically-active agent within its approved dosage range.

According to this aspect of the invention there is provided a combination for use in the treatment of a cancer (for example a cancer involving a solid tumour) comprising a compound of the invention as defined herein, or a pharmaceutically acceptable salt, hydrate or solvate thereof, and another anti-tumour agent.

According to this aspect of the invention there is provided a combination for use in the treatment of a proliferative condition, such as, but not limited to, cancer (for example a cancer involving a solid tumour), comprising a compound of the invention as defined herein, or a pharmaceutically acceptable salt, hydrate or solvate thereof, and any one of the anti-tumour agents listed herein above.

In a further aspect of the invention there is provided a compound of the invention or a pharmaceutically acceptable salt, hydrate or solvate thereof, for use in the treatment of cancer in combination with another anti-tumour agent, optionally selected from one listed herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination. In one embodiment, a combination refers to a combination product.

According to a further aspect of the invention there is provided a pharmaceutical composition which comprises a compound of the invention, or a pharmaceutically acceptable salt, hydrate or solvate thereof, in combination with an anti-tumour agent (optionally selected from one listed herein above), in association with a pharmaceutically acceptable diluent or carrier.

### EXAMPLES

The invention will now be illustrated, but not limited, by reference to the specific embodiments described in the following examples. Compounds are named using conventional IUPAC nomenclature, or as named by the chemical supplier.

The following synthetic procedures are provided for illustration of the methods used; for a given preparation or step the precursor used may not necessarily derive from the individual batch synthesized according to the step in the description given.

### Analytical Methods (AM)

Where examples and preparations cite analytical data, the following analytical methods were used unless otherwise specified.

All LCMS spectra were obtained by using one of the below methods.

Method 3 (AM3): (5-95 A-B_1.55 min_220 & 254 nm): Instrument: SHIMADZU LCMS-2020; Column: Kinetex EVO C18 30×2.1 mmx 5 μm; Run Time: 1.55 min; Solvents: A) 0.0375% TFA in water (v/v), B) 0.01875% TFA in acetonitrile (v/v). The gradient runs with 5% B; Gradient: 5-95% B with A, 0.8 min; hold at 95% B to 1.2 min; 5% B at 1.21 min and hold at 5% B to 1.55 min® 1.5 mL/min, 50° C.

Method 7 (AM7): (5-95 C-D_1.5 mn_RE220&254_POS): Instrument: SHIMADZU LCMS-2020; Column: Kinetex EVO Ce8 30×2.1 mmx 5 m; Run Time: 1.5 m; Solvents A) 0.025% NH3·H2O in water (v/v) B) Acetonitrile. The gradient runs with 5% B. Gradient: 5-95% B with A 0.8 min, hold at 95% B to 1.2 min; 5% B at 1.21 min and hold at 5% B to 1.5 min® 1.5 mi/min, 40° C.

¹H NMR spectra were acquired on a Bruker Avance III spectrometer at 400 MHz using residual undeuterated solvent as reference. The spectra were processed using interpretation software ACID Spectrus processor or equivalent software.

### Purification Methods (PM)

Chromatography

| Purification | | | Eluent |
|---|---|---|---|
| Method | Column | Eluent | Ratio |
| 1 | SiO₂ | petroleum ether:ethyl | 1:1 |
| | | acetate | |
| 2 | SiO₂ | petroleum ether:ethyl | 2:1 |
| | | acetate | |
| 3 | SiO₂ | petroleum ether:ethyl | 5:1 |
| | | acetate | |
| 4 | SiO₂ | petroleum ether:ethyl | 13:1 |
| | | acetate | |
| 5 | SiO₂ | petroleum ether:ethyl | 40:1 |
| | | acetate | |
| 6 | SiO₂ | ethyl acetate | 1 |
| 7 | SiO₂ | petroleum ether:ethyl | 5:1 to 2:1 |
| | | acetate | |
| 8 | SiO₂ | petroleum ether:ethyl | 10:1 to 3:1 |
| | | acetate | |

Reverse-phase HPLC conditions

| Purifi- | | | |
|---|---|---|---|
| cation | | | |
| method | Column | Mobile phase | Gradient |
| 9 | Phenomenex Synergi C18 | water (0.1% FA)-ACN] | 10%-90%, 20 min |
| | 150 × 25 mm × 10 mm | | |
| 10 | Phenomenex Gemini-NX C18 | water (0.225% FA)-ACN | 70%-100%, 7 min |
| | 75 × 30 mm × 3 mm | | |
| 11 | Phenomenex Gemini-NX C18 | water (0.225% FA)-ACN | 32%-62%, 7 min |
| | 75 × 30 mm × 3 mm | | |
| 12 | Waters Xbridge | water (10 mM | 22%-52%, 11 min |
| | 150 × 25 mm × 5 mm | NH₄HCO₃)-ACN | |
| 13 | Phenomenex Gemini-NX C18 | water (0.225% FA)-ACN | 38%-48%, 7 min |
| | 75 × 30 mm × 3 mm | | |
| 14 | Phenomenex Synergi C18 | water (0.1% ammonium | 95%-100%, 10 min |
| | 150 × 25 mm × 10 mm | hydroxide v/v)-ACN | |
| 15 | Phenomenex Gemini-NX C18 | water (0.225% FA)-ACN | 35%-45%, 7 min |
| | 75 × 30 mm × 3 mm | | |
| 16 | Waters Xbridge | water (0.05% ammonium | 68%-98%, 8 mins |
| | 150 × 25 mm × 5 mm | hydroxide v/v)-ACN | |
| 17 | Phenomenex Gemini-NX C18 | water (0.05% ammonium | 66%-96%, 7 mins |
| | 75 × 30 mm × 3 mm | hydroxide v/v)-ACN | |
| 18 | Phenomenex luna C18 | water (0.2% FA)-ACN | 24%-44%, 12 min |
| | 150 × 25 mm × 10 mm | | |
| 19 | Waters Xbridge | water (10 mM | 60%-90%, 7 min |
| | 150 × 25 mm × 5 mm | NH4HCO3)-ACN | |
| 20 | Phenomenex Gemini-NX C18 | water (0.225% FA)-ACN | 25%-55%, 5 min |
| | 75 × 30 mm × 3 mm | | |
| 21 | Phenomenex Synergi C18 | water (0.1% ammonium | 50%-65%, 15 min |
| | 150 × 25 mm × 10 mm | hydroxide v/v)-ACN | |
| 22 | Unisil 3-100 C18 Ultra | water (0.225% FA)-ACN | 30%-60%, 10 min |
| | 150 × 50 mm × 3 mm | | |
| 23 | Waters Xbridge | water (0.05% ammonium | 58%-88%, 8 mins |
| | 150 × 25 mm × 5 mm | hydroxide v/v)-ACN | |
| 24 | Phenomenex Synergi C18 | water (0.1% FA)-ACN] | 45%-70%, 20 min |
| | 150 × 25 mm × 10 mm | | |
| 25 | Waters Xbridge | water (0.05% ammonium | 62%-92%, 8 mins |
| | 150 × 25 mm × 5 mm | hydroxide v/v)-ACN | |
| 26 | Phenomenex Gemini-NX C18 | water (0.225% FA)-ACN | 32%-42%, 7 min |
| | 75 × 30 mm × 3 mm | | |
| 27 | Waters Xbridge | water (0.05% ammonium | 64%-94%, 8 mins |
| | 150 × 25 mm × 5 mm | hydroxide v/v)-ACN | |
| 28 | Waters Xbridge | water (0.05% ammonium | 66%-96%, 8 mins |
| | 150 × 25 mm × 5 mm | hydroxide v/v)-ACN | |
| 29 | Phenomenex Synergi C18 | water (0.1% ammonium | 70%-95%, 15 min |
| | 150 × 25 mm × 10 mm | hydroxide v/v)-ACN | |
| 30 | Phenomenex Gemini-NX C18 | water (0.225% FA)-ACN | 22%-52%, 5 min |
| | 75 × 30 mm × 3 mm | | |
| 31 | Phenomenex Gemini-NX C18 | water (0.225% FA)-ACN | 22%-52%, 5 min |
| | 75 × 30 mm × 3 mm | | |

### ABBREVIATIONS

Wherein the following abbreviations have been used, the following meanings apply:
ACN is acetonitrile,
AcOH is acetic acid,
AM is analytical method,
aq. is aqueous,
atm. is atmosphere,
tBuOH is tert-Butyl alcohol,
BOC₂O is Di-tert-butyl dicarbonate,
CHCl₃-d is deuterated chloroform,
DCM is dichloromethane,
DIPEA is N,N-diisopropylethylamine,
DMAP is 4-Dimethylaminopyridine,
DME is 1,2-dimethoxyethane,
DMF is N,N-dimethylformamide,
DMS is dimethyl sulfide,
DMSO is dimethyl sulfoxide,
DMSO-d₆ is dimethyl sulfoxide,
EA is ethyl acetate,
EtOH is ethanol,
FA is formic acid,
h is Hours,
O₃ is Ozone,
HCl is hydrochloric acid,
H₂O is water,
HPLC is high performance liquid chromatography,
K₂CO₃ is potassium carbonate,
LAH is lithium aluminum hydride,
LCMS is Liquid Chromatography Mass Spectrometry,
LiOH·H₂O is lithium hydroxide monohydrate,
MeCN is Acetonitrile,
MeOH is methanol,
MeOH-d₄ is deuterated methanol,
min is minutes,
N₂ is nitrogen gas,
NH₄OH is ammonium hydroxide,
NaHCO₃ is sodium bicarbonate,
NaH is sodium hydride,
NaOH is sodium hydroxide,
NaB(OAc)₃H is sodium triacetoxyborohydride
NaBH₄ is sodium borohydride
Na₂SO₄ is anhydrous sodium sulfate,
NH₄HCO₃ is Ammonium bicarbonate,
NMR is nuclear magnetic resonance,
° C. is Celsius scale,
Pd(dppf)Cl₂ is [1,1′-Bis(diphenylphosphino)ferrocene]dichloropalladium(II),
PE is Petroleum ether,
PM is purification method,
rt is retention time,
SiO₂ is Silica,
TEA is triethylamine,
TFA is trifluoroacetic acid,
THE is tetrahydrofuran and
TLC is thin layer chromatography.

### Preparation of Intermediates

The following Preparations describe the methods used for common intermediates required for synthesis of the Examples.

### Synthesis of Intermediate Q

### tert-butyl (4-(allyloxy)butyl)carbamate, 1.53

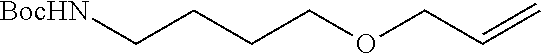 [SMILES: C=CCOCCCCNC(=O)OC(C)(C)C]

To a solution of NaOH (2.11 g, 52.84 mmol) in 1,4-dioxane (176.1 mL) was added tert-butyl N-(4-hydroxybutyl)carbamate (10 g, 52.84 mmol) and 3-bromoprop-1-ene (12.78 g, 105.68 mmol), sequentially at 20° C. The reaction mixture was heated to 70° C. and stirred for 12 h. The reaction mixture was diluted with H₂O (100 mL) and extracted with EA (100 mL×3). The combined organic layer was washed, brine (80 mL×2), dried over anhydrous Na₂SO₄, filtered and concentrated in vacuo. The residue was purified (PM3) to afford compound 1.53 (5.5 g, 23.98 mmol, 45.4% yield) as a light yellow oil.

¹H NMR (400 MHz, CHCl₃-d) δ: 5.93-5.82 (m, 1H), 5.27-5.20 (m, 1H), 5.16-5.11 (m, 1H), 4.70 (br, s, 1H), 3.93-3.91 (m, 2H), 3.43-3.39 (t, 2H), 3.12-3.08 (m, 2H), 1.62-1.49 (m, 4H), 1.40 (s, 9H) ppm.

### tert-butyl (4-(2-hydroxyethoxy)butyl)carbamate, 1.54

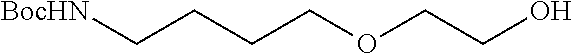 [SMILES: CC(C)(C)OC(=O)NCCCCOCCO]

O₃ was bubbled into a solution of compound 1.53 (5.5 g, 23.98 mmol) in DCM (50 mL) at −78° C. until the mixture turned blue, the reaction mixture was warmed to 0° C. and NaBH₄ (1.77 g, 46.79 mmol) was added slowly. The reaction mixture was warmed to 20° C. and stirred for 12 h. The reaction was quenched with H₂O (50 mL), extracted with DCM (80 mL×2), the combined organic layer was washed, brine (80 mL×2), dried over anhydrous Na₂SO₄, then filtered and concentrated in vacuo. The residue was purified (PM2) to afford compound 1.54 (2.65 g, 11.36 mmol, 47.4% yield) as a colorless oil.

¹H NMR (400 MHz, CHCl₃-d) δ: 4.78 (br s, 1H), 3.72-3.71 (m, 2H), 3.53-3.51 (t, 2H), 3.51-3.46 (t, 2H), 3.13-3.12 (m, 2H), 2.41 (br s, 1H), 1.66-1.50 (m, 4H), 1.42 (s, 9H) ppm.

### 5-(2-(4-((tert-butoxycarbonyl)amino)butoxy)ethoxy)benzo[c][2,6]naphthyridine-8-carboxylic acid, 1.55

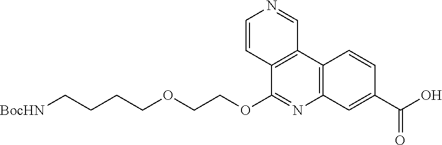 [SMILES: CC(C)(C)OC(=O)NCCCCOCCOc1nc2cc(C(=O)O)ccc2c2cnccc12]

To a mixture of compound 1.54 (427.79 mg, 1.83 mmol) in DMF (10 mL) was added NaH (110.02 mg, 2.75 mmol), followed by compound 1.1 (500 mg, 1.83 mmol), under N₂ protection at 0° C. The mixture was heated to 80° C. and stirred for 12 h. The mixture was diluted with H₂O (50 mL), extracted with EA (50 mL×2), the combined organic phases washed with brine (50 mL), dried over Na₂SO₄ and concentrated in vacuo. The residue was purified (PM5) to afford compound 1.55 (300 mg, 645.44 mol, 35.2% yield, 98.2% purity) as a light yellow solid.

LCMS (AM3): rt=0.903 min, (456.3 [M+H]⁺), 98.2% purity.

### 5-(2-(4-Aminobutoxy)ethoxy)benzo[c][2,6]naphthyridine-8-carboxylic acid; Intermediate Q

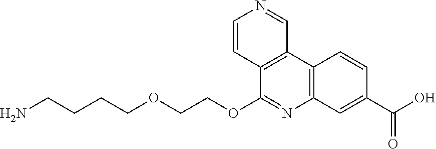 [SMILES: NCCCCOCCOc1nc2cc(C(=O)O)ccc2c2cnccc12]

To a solution of compound 1.55 (100 mg, 219.54 μmol) in DCM (5 mL) was added TFA (1 mL, 13.51 mmol). The mixture was stirred at 25° C. for 0.5 h. The mixture was concentrated in vacuo to afford Intermediate Q (100 mg, 213.03 mol, 97% yield, TFA salt) as a brown solid, which was used without purification.

LCMS (AM3): rt=0.745 min, (356.3 [M+H]⁺), 79.9% purity

### Synthesis of Intermediate 1.507

### 1,3-Difluoro-2-(trifluoromethoxy)-5-vinylbenzene 1.506

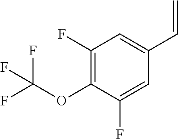

To a solution of 4,4,5,5-tetramethyl-2-vinyl-1,3,2-dioxaborolane (6.26 g, 40.62 mmol) and 5-bromo-1,3-difluoro-2-(trifluoromethoxy)benzene (7.5 g, 27.08 mmol) in DME (100 mL) was added Pd(dppf)Cl₂ (1.98 g, 2.71 mmol) and CsF (8.23 g, 54.15 mmol). The mixture was heated to 80° C. and stirred for 12 h under N₂. The mixture was poured into H₂O (300 mL) and the aqueous phase was extracted with EA (150 mL×3). The combined organic phase was washed, brine (300 mL×2), dried, anhydrous Na₂SO₄, filtered and concentrated in vacuo. The residue was purified (PM4) to give compound 1.506 (3.2 g, 14.28 mmol, 52.7% yield) as a yellow oil.

¹H NMR (400 MHz, CHCl₃-d) δ: 7.05 (s, 1H), 7.03 (s, 1H), 6.61 (dd, J=17.6, 10.8 Hz, 1H), 5.78 (d, J=17.2 Hz, 1H), 5.43 (d, J=10.8 Hz, 1H) ppm.

### 3,5-Difluoro-4-(trifluoromethoxy)benzaldehyde 1.507

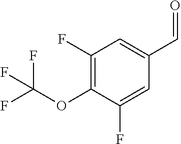 [SMILES: O=Cc1cc(F)c(OC(F)(F)F)c(F)c1]

O₃ was bubbled into a solution of compound 1.506 (3.2 g, 14.28 mmol) in DCM (40 mL) at −70° C. until the reaction mixture turned blue, excess O₃ was purged and DMS (8.87 g, 142.78 mmol) was added. The mixture was warmed up to 20° C. and stirred for 12 h. The mixture was concentrated in vacuo and purified (PM3) to give compound 1.507 (1.2 g, 5.31 mmol, 37.2% yield) as a yellow oil.

¹H NMR (400 MHz, CHCl₃-d) δ: 9.95 (s, 1H), 7.59 (s, 1H), 7.57 (s, 1H) ppm.

### Synthesis of Intermediate 1.839

### 5-(2-(4-((Tert-butoxycarbonyl)(3,5-difluoro-4-(trifluoromethoxy)benzyl)amino)butoxy)ethoxy)benzo[c][2,6]naphthyridine-8-carboxylic acid 1.839

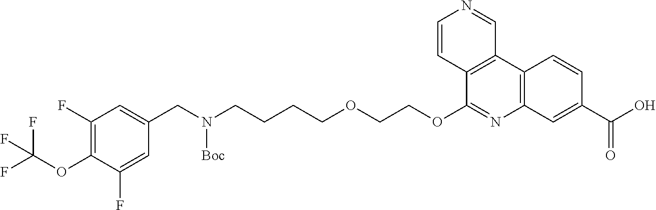 [SMILES: CC(C)(C)OC(=O)N(CCCCOCCOc1nc2cc(C(=O)O)ccc2c2cnccc12)Cc1cc(F)c(OC(F)(F)F)c(F)c1]

To a solution of REFERENCE EXAMPLE 1 (3 g, 4.91 mmol, FA salt) in EtOH (30 mL) was added DIPEA (1.86 g, 14.35 mmol) and Boc₂O (1.43 g, 6.53 mmol) at 20° C. The reaction mixture was stirred at 20° C. for 12 h. The reaction mixture was concentrated in vacuo to give a residue, which was purified (PM9) to afford compound 1.839 (2.6 g, 3.01 mmol, 61.42% yield) as white solid.

LCMS (AM3): rt=1.076 min, (666.1 [M+H]⁺), 77.1% purity.

### Synthesis of Intermediate 2.046

### ethyl 5-(2-(4-((tert-butoxycarbonyl)(3,5-difluoro-4-(trifluoromethoxy)benzyl)amino)butoxy)ethoxy)benzo[c][2,6]naphthyridine-8-carboxylate 2.046

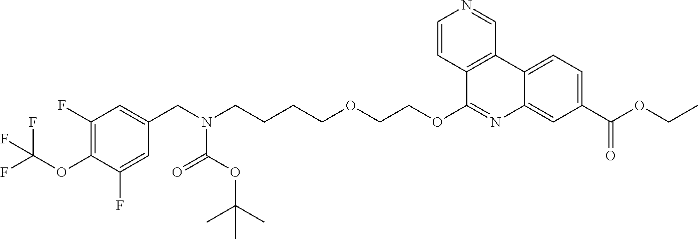 [SMILES: CCOC(=O)c1ccc2c(c1)nc(OCCOCCCCN(Cc1cc(F)c(OC(F)(F)F)c(F)c1)C(=O)OC(C)(C)C)c1ccncc12]

A mixture of REFERENCE EXAMPLE 1 (200 mg, 353.68 μmol), DIPEA (1.15 mmol, 0.2 mL), DMAP (10 mg, 81.85 mol) and Boc₂O (1.74 mmol, 0.4 mL) in EtOH (2 mL) was stirred at 30° C. for 16 hours. Boc₂O (1.74 mmol, 0.4 mL) was added and the mixture was stirred for 2 hours. The reaction mixture was concentrated in vacuo to give a residue, which was purified (PM7) to afford compound 2.046 (200 mg, 81.12% yield, 99.5% purity) as colorless oil.

LCMS (AM3): rt=1.215 min, (694.5 [M+H]⁺), 99.52% purity.

### Synthesis of Intermediate 2.047

### tert-butyl 5-(2-(4-((tert-butoxycarbonyl)(3,5-difluoro-4-(trifluoromethoxy)benzyl)amino)butoxy)ethoxy)benzo[c][2,6]naphthyridine-8-carboxylate 2.047

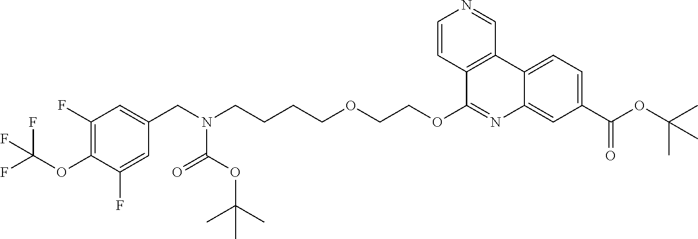 [SMILES: CC(C)(C)OC(=O)c1ccc2c(c1)nc(OCCOCCCCN(Cc1cc(F)c(OC(F)(F)F)c(F)c1)C(=O)OC(C)(C)C)c1ccncc12]

A mixture of REFERENCE EXAMPLE 1 (200 mg, 353.68 μmol), DIPEA (1.15 mmol, 0.2 mL), DMAP (5 mg, 40.93 μmol) and Boc₂O (1.74 mmol, 0.4 mL) in t-BuOH (1 mL) and THE (1 mL) was stirred at 30° C. for 1 hour, then stirred at 60° C. for 17 hours. The reaction mixture was concentrated in vacuo to give a residue, which was purified (PM8) to afford compound 2.047 (200 mg, 75.22% yield) as a colorless oil.

LCMS (AM3): rt=1.267 min, (722.6 [M+H]⁺), 96.32% purity.

### Synthesis of Intermediate 2.048

### acetoxymethyl 5-(2-(4-((tert-butoxycarbonyl)(3,5-difluoro-4-(trifluoromethoxy)benzyl)amino)butoxy)ethoxy)benzo[c][2,6]naphthyridine-8-carboxylate 2.048

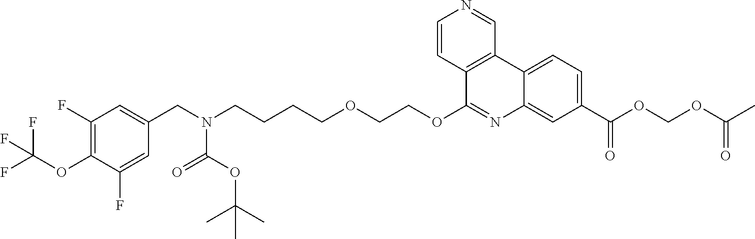 [SMILES: CC(=O)OCOC(=O)c1ccc2c(c1)nc(OCCOCCCCN(Cc1cc(F)c(OC(F)(F)F)c(F)c1)C(=O)OC(C)(C)C)c1ccncc12]

A mixture of compound 1.839 (200 mg, 300.48 μmol), bromomethyl acetate (90 mg, 588.33 mol) and DIPEA (574.13 μmol, 0.1 mL) in DMF (2 mL) was stirred at 80° C. for 2 hours. EA (40 mL) was added, the mixture washed with water (20 mL×2), brine (20 mL), dried over Na₂SO₄, filtered and concentrated in vacuo to give a residue, which was purified (PM7) to afford compound 2.048 (120 mg, 54.62% yield) as yellow oil.

LCMS (AM3): rt=1.174 min, (738.3 [M+H]⁺), 65.58% purity.

### Synthesis of Intermediate 2.049

### ((methoxycarbonyl)oxy)methyl 5-(2-(4-((tert-butoxycarbonyl)(3,5-difluoro-4-(trifluoromethoxy)benzyl)amino)butoxy)ethoxy)benzo[c][2,6]naphthyridine-8-carboxylate 2.049

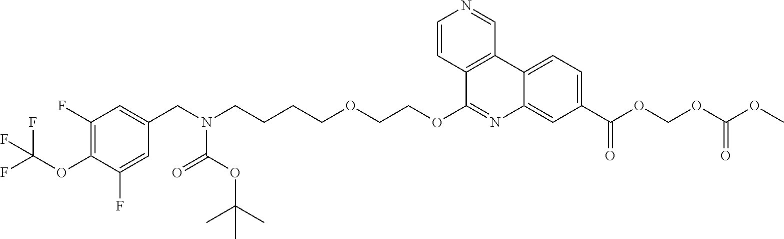 [SMILES: COC(=O)OCOC(=O)c1ccc2c(c1)nc(OCCOCCCCN(Cc1cc(F)c(OC(F)(F)F)c(F)c1)C(=O)OC(C)(C)C)c1ccncc12]

A mixture of compound 1.839 (200 mg, 300.48 mol), chloromethyl methyl carbonate (41 mg, 329.26 μmol) and DIPEA (918.59 mol, 0.16 mL) in DMF (2 mL) was stirred at 80° C. for 2 hours. The reaction was diluted with EA (40 mL), washed with water (20 mL×2), brine (20 mL), dried over Na₂SO₄, filtered and concentrated in vacuo to give a residue, which was purified (PM7) to afford compound 2.049 (180 mg, 78.89% yield) as a yellow oil.

LCMS (AM3): rt=1.183 min, (754.3 [M+H]⁺), 99.26% purity.

### Synthesis of Intermediate 2.050

### (propionyloxy)methyl 5-(2-(4-((tert-butoxycarbonyl)(3,5-difluoro-4-(trifluoromethoxy)benzyl)amino)butoxy)ethoxy)benzo[c][2,6]naphthyridine-8-carboxylate 2.050

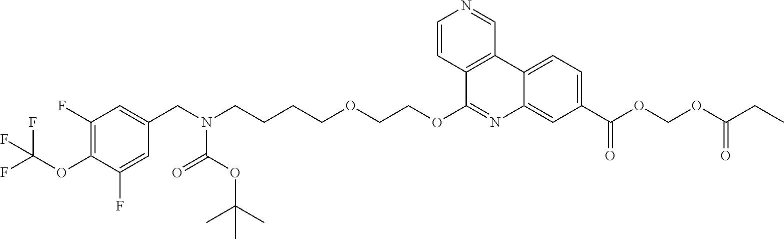 [SMILES: CCC(=O)OCOC(=O)c1ccc2c(c1)nc(OCCOCCCCN(Cc1cc(F)c(OC(F)(F)F)c(F)c1)C(=O)OC(C)(C)C)c1ccncc12]

A mixture of compound 1.839 (200 mg, 300.48 μmol), chloromethyl propanoate (55 mg, 448.80 μmol) (Ref: WO2019/133770) and DIPEA (918.59 mol, 0.16 mL) in DMF (2 mL) was stirred at 80° C. for 2 hours. The reaction mixture was diluted with EA (40 mL), the mixture washed with water (20 mL×2), brine (20 mL), dried over Na₂SO₄, filtered and concentrated in vacuo to give a residue, which was purified (PM7) to afford compound 2.050 (140 mg, 61.98% yield) as colorless oil.

LCMS (AM3): rt=1.209 min, (752.3 [M+H]⁺), 100% purity.

### Synthesis of Intermediate 2.051

### ((tert-butoxycarbonyl)oxy)methyl 5-(2-(4-((tert-butoxycarbonyl)(3,5-difluoro-4-(trifluoromethoxy)benzyl)amino)butoxy)ethoxy)benzo[c][2,6]naphthyridine-8-carboxylate 2.051

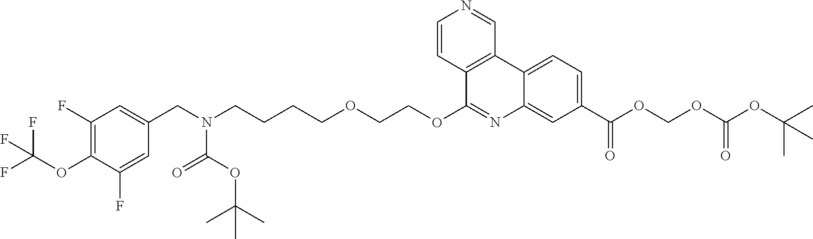 [SMILES: CC(C)(C)OC(=O)OCOC(=O)c1ccc2c(c1)nc(OCCOCCCCN(Cc1cc(F)c(OC(F)(F)F)c(F)c1)C(=O)OC(C)(C)C)c1ccncc12]

A mixture of compound 1.839 (400 mg, 600.96 μmol), tert-butyl chloromethyl carbonate (120 mg, 720.28 mol) (Ref: Tetrahedron Letters, 2007, vol. 48, #1, p. 109-112) and DIPEA (1.80 mmol, 0.32 mL) in DMF (10 mL) was stirred at 80° C. for 14 hours. The reaction mixture was diluted with EA (40 mL), washed with water (20 mL×2), brine (20 mL), dried over Na₂SO₄, filtered and concentrated in vacuo to give a residue, which was purified (PM7) to afford compound 2.051 (350 mg, 71.32% yield) as light yellow oil.

LCMS (AM3): rt=1.251 min, (796.3 [M+H]⁺), 97.45% purity.

### Synthesis of Intermediate 2.052

### isopropyl 5-(2-(4-((tert-butoxycarbonyl)(3,5-difluoro-4-(trifluoromethoxy)benzyl)amino)butoxy)ethoxy)benzo[c][2,6]naphthyridine-8-carboxylate 2.052

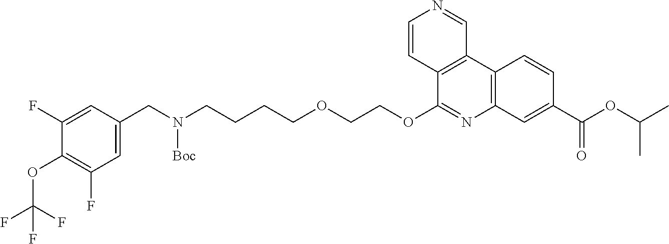 [SMILES: CC(C)OC(=O)c1ccc2c(c1)nc(OCCOCCCCN(Cc1cc(F)c(OC(F)(F)F)c(F)c1)C(=O)OC(C)(C)C)c1ccncc12]

To a solution of compound 1.839 (200 mg, 300.48 μmol) in DMF (2 mL) was added K₂CO₃ (83.06 mg, 600.96 mol) and 2-iodopropane (51.08 mg, 300.48 μmol), the reaction mixture was stirred at 50° C. for 2 hours. The reaction mixture was filtered and the filtrate was concentrated in vacuo to give the residue, which was purified (PM10) to afford compound 2.052 (100 mg, 141.31 μmol, 47.03% yield) as colorless oil.

LCMS (AM3): rt=1.199 min, (708.2 [M+H]⁺), 91.6% purity.

### Synthesis of Intermediate 2.053

### 2-(dimethylamino)ethyl 5-(2-(4-((tert-butoxycarbonyl)(3,5-difluoro-4-(trifluoromethoxy)benzyl)amino)butoxy)ethoxy)benzo[c][2,6]naphthyridine-8-carboxylate 2.053

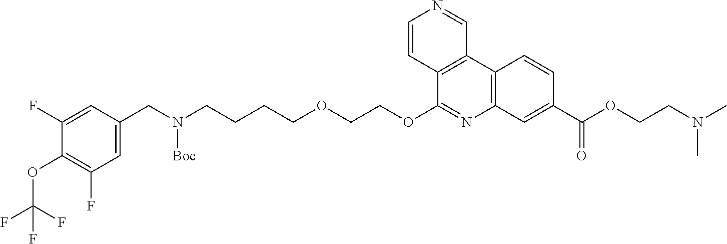 [SMILES: CN(C)CCOC(=O)c1ccc2c(c1)nc(OCCOCCCCN(Cc1cc(F)c(OC(F)(F)F)c(F)c1)C(=O)OC(C)(C)C)c1ccncc12]

To a solution of compound 1.839 (200 mg, 300.48 μmol) in DMF (2 mL) was added K₂CO₃ (124.58 mg, 901.44 μmol) and 2-chloro-N,N-dimethylethan-1-amine hydrogen chloride (43.28 mg, 300.48 μmol). The reaction mixture was stirred at 50° C. for 2 hours. The reaction mixture was filtered and the filtrate was concentrated in vacuo to give the filtrate, which was purified (PM11) to afford compound 2.053 (100 mg, 135.74 mol, 45.17% yield) as yellow oil.

LCMS (AM3): rt=0.971 min, (737.2 [M+H]⁺), 97.3% purity.

### Synthesis of Intermediate 2.054

### methyl 5-(2-(4-((tert-butoxycarbonyl)(3,5-difluoro-4-(trifluoromethoxy)benzyl)amino)butoxy)ethoxy)benzo[c][2,6]naphthyridine-8-carboxylate 2.054

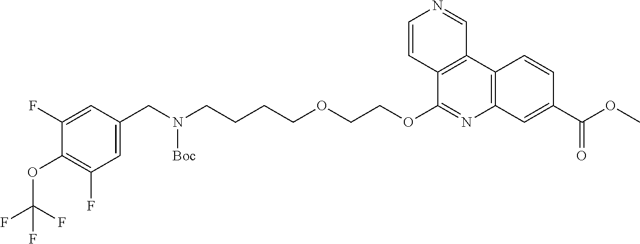 [SMILES: COC(=O)c1ccc2c(c1)nc(OCCOCCCCN(Cc1cc(F)c(OC(F)(F)F)c(F)c1)C(=O)OC(C)(C)C)c1ccncc12]

To a solution of REFERENCE EXAMPLE 1 (200 mg, 353.68 μmol) in THE (2 mL) was added Boc₂O (380.00 mg, 1.74 mmol), DIPEA (148.40 mg, 1.15 mmol) and DMAP (4.32 mg, 35.37 mol). The reaction mixture was stirred at 30° C. for 1 hour. MeOH (791.80 mg, 24.71 mmol) was added. The reaction mixture was concentrated in vacuo to give a residue, which was purified (P10) to afford compound 2.054 (100 mg, 147.14 μmol, 41.60% yield) as a yellow solid.

LCMS (AM3): rt=1.163 min, (680.1 [M+H]⁺), 99.6% purity.

### Synthesis of Intermediate 2.055

### (isobutyryloxy)methyl 5-(2-(4-((tert-butoxycarbonyl)(3,5-difluoro-4-(trifluoromethoxy)benzyl)amino)butoxy)ethoxy)benzo[c][2,6]naphthyridine-8-carboxylate 2.055

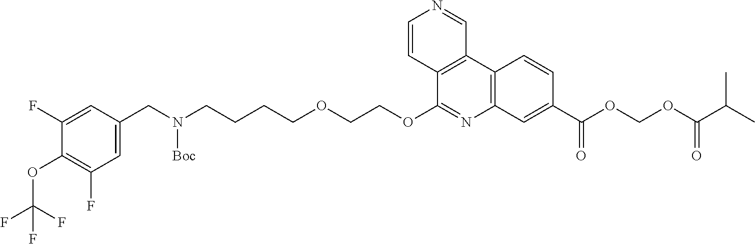 [SMILES: CC(C)C(=O)OCOC(=O)c1ccc2c(c1)nc(OCCOCCCCN(Cc1cc(F)c(OC(F)(F)F)c(F)c1)C(=O)OC(C)(C)C)c1ccncc12]

To a solution of compound 1.839 (200 mg, 300.48 μmol) in MeCN (2.5 mL) was added chloromethyl isobutyrate (45.14 mg, 330.53 μmol) and K₂CO₃ (83.06 mg, 600.96 μmol). The reaction mixture was stirred at 80° C. for 3 hours. Chloromethyl isobutyrate (41.04 mg, 300.48 μmol) and K₂CO₃ (41.53 mg, 300.48 μmol) was added and the reaction was stirred at 80° C. for 12 hours. The reaction mixture was filtered and the filtrate was concentrated in vacuo to give a residue, which was purified (PM6) to afford compound 2.055 (170 mg, 222.01 μmol, 73.89% yield) as a yellow oil.

LCMS (AM3): rt=1.237 min, (766.4 [M+H]⁺), 71.9% purity.

### Synthesis of Intermediate 2.056

### (pivaloyloxy)methyl 5-(2-(4-((tert-butoxycarbonyl)(3,5-difluoro-4-(trifluoromethoxy)benzyl)amino)butoxy)ethoxy)benzo[c][2,6]naphthyridine-8-carboxylate 2.056

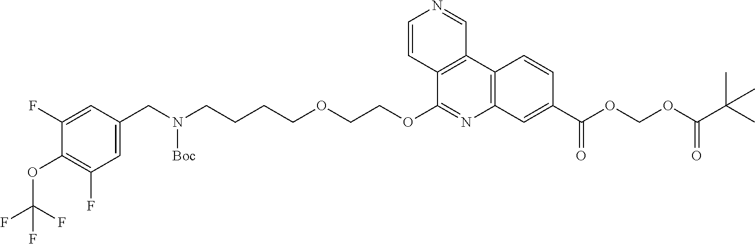 [SMILES: CC(C)(C)OC(=O)N(CCCCOCCOc1nc2cc(C(=O)OCOC(=O)C(C)(C)C)ccc2c2cnccc12)Cc1cc(F)c(OC(F)(F)F)c(F)c1]

To a solution of compound 1.839 (200 mg, 300.48 mol) in MeCN (3 mL) was added iodomethyl pivalate (80.01 mg, 330.53 μmol) and K₂CO₃ (83.06 mg, 600.96 μmol). The reaction mixture was stirred at 80° C. for 15 hours. The reaction mixture was concentrated in vacuo to give a residue, which was purified (PM6) to afford compound 2.056 (70 mg, 84.44 μmol, 28.10% yield) as a yellow oil.

LCMS (AM3): rt=1.222 min, (780.1 [M+H]⁺), 94.1% purity.

### Synthesis of Intermediate 2.057

### (5-methyl-2-oxo-1,3-dioxol-4-yl)methyl 5-(2-(4-((tert-butoxycarbonyl)(3,5-difluoro-4-(trifluoromethoxy)benzyl)amino)butoxy)ethoxy)benzo[c][2,6]naphthyridine-8-carboxylate 2.057

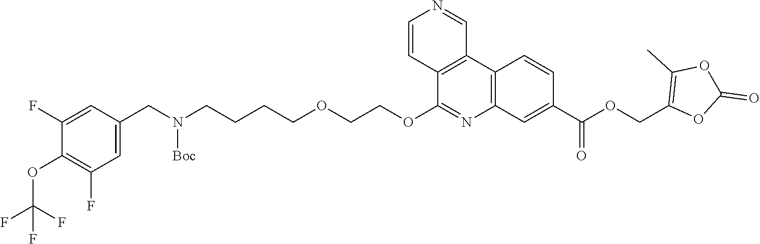 [SMILES: Cc1oc(=O)oc1COC(=O)c1ccc2c(c1)nc(OCCOCCCCN(Cc1cc(F)c(OC(F)(F)F)c(F)c1)C(=O)OC(C)(C)C)c1ccncc12]

To a solution of compound 1.839 (200 mg, 300.48 μmol) in DMF (2 mL) was added 4-(bromomethyl)-5-methyl-1,3-dioxol-2-one (63.79 mg, 330.53 μmol) and DIPEA (77.67 mg, 600.96 μmol). The reaction mixture was stirred at 80° C. for 3 hours. The reaction mixture was diluted with H₂O (40 mL) and extracted with EA (20 mL×2). The combined organic layers were washed with brine (50 mL), dried over Na₂SO₄, filtered and concentrated in vacuo to give a residue, which was purified (PM1) to afford compound 2.057 (220 mg, 282.89 μmol, 94.15% yield) as a yellow oil.

LCMS (AM3): rt=1.158 min, (778.3 [M+H]⁺), 100% purity.

### Synthesis of Intermediate 2.058

### ((ethoxycarbonyl)oxy)methyl 5-(2-(4-((tert-butoxycarbonyl)(3,5-difluoro-4-(trifluoromethoxy)benzyl)amino)butoxy)ethoxy)benzo[c][2,6]naphthyridine-8-carboxylate 2.058

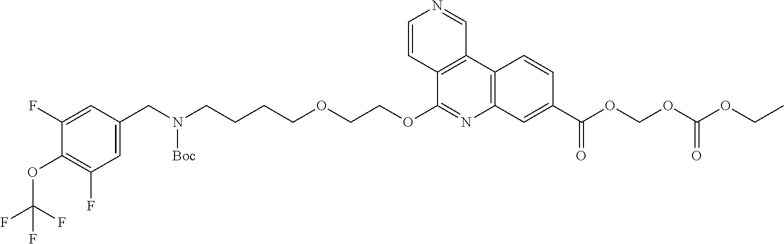 [SMILES: CCOC(=O)OCOC(=O)c1ccc2c(c1)nc(OCCOCCCCN(Cc1cc(F)c(OC(F)(F)F)c(F)c1)C(=O)OC(C)(C)C)c1ccncc12]

To a solution of compound 1.839 (200 mg, 300.48 μmol) in DMF (2 mL) was added DIPEA (77.67 mg, 600.96 μmol) and chloromethyl ethyl carbonate (45.79 mg, 330.53 μmol). The reaction mixture was stirred at 80° C. for 12 hours. The reaction mixture was diluted with H₂O (30 mL) and extracted with EA (20 mL×2). The combined organic layers were washed with brine (50 mL), dried over Na₂SO₄, filtered and concentrated in vacuo to give a residue, which was purified (PM1) to afford compound 2.058 (200 mg, 260.52 μmol, 86.70% yield) as yellow oil.

LCMS (AM3): rt=1.167 min, (768.4 [M+H]⁺), 99.0% purity.

### Synthesis of Intermediate 2.059

### ((isopropoxycarbonyl)oxy)methyl 5-(2-(4-((tert-butoxycarbonyl)(3,5-difluoro-4-(trifluoromethoxy)benzyl)amino)butoxy)ethoxy)benzo[c][2,6]naphthyridine-8-carboxylate 2.059

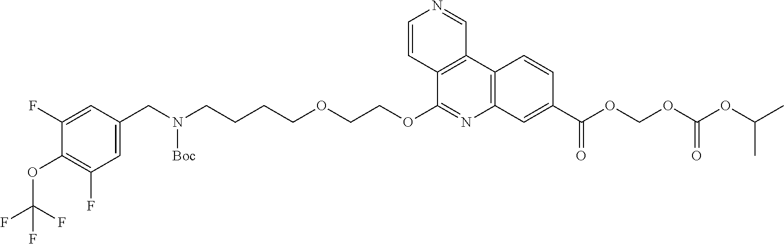 [SMILES: CC(C)OC(=O)OCOC(=O)c1ccc2c(c1)nc(OCCOCCCCN(Cc1cc(F)c(OC(F)(F)F)c(F)c1)C(=O)OC(C)(C)C)c1ccncc12]

To a solution of compound 1.839 (200 mg, 300.48 μmol) in DMF (2 mL) was added DIPEA (77.67 mg, 600.96 μmol) and chloromethyl isopropyl carbonate (50.43 mg, 330.53 μmol). The reaction mixture was stirred at 80° C. for 12 hours. The reaction mixture was diluted with H₂O (20 mL) and extracted with EA (30 mL×2). The combined organic layers were washed with brine (40 mL), dried over Na₂SO₄, filtered and concentrated in vacuo to give a residue, which was purified (PM1) to afford compound 2.059 (160 mg, 202.77 μmol, 67.48% yield) as yellow oil.

LCMS (AM3): rt=1.200 min, (782.3 [M+H]⁺), 98.7% purity.

### Synthesis of Intermediate 2.061

### ((((tetrahydro-2H-pyran-4-yl)oxy)carbonyl)oxy)methyl 5-(2-(4-((tert-butoxycarbonyl)(3,5-difluoro-4-(trifluoromethoxy)benzyl)amino)butoxy)ethoxy)benzo[c][2,6]naphthyridine-8-carboxylate 2.061

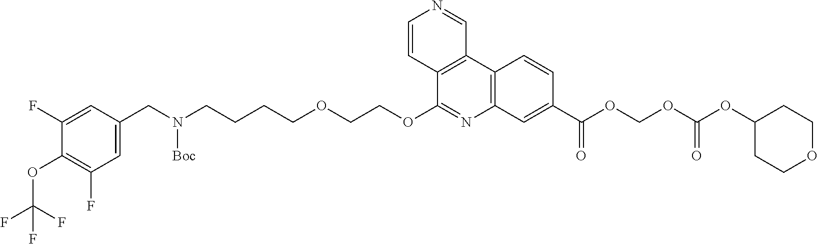 [SMILES: CC(C)(C)OC(=O)N(CCCCOCCOc1nc2cc(C(=O)OCOC(=O)OC3CCOCC3)ccc2c2cnccc12)Cc1cc(F)c(OC(F)(F)F)c(F)c1]

To a solution of compound 1.839 (200 mg, 300.48 μmol) in DMF (3 mL) was added DIPEA (116.50 mg, 901.44 μmol) and compound 2.060 (116.95 mg, 600.96 μmol) (Ref: WO2015/165833). The reaction mixture was stirred at 80° C. for 3 hours. The reaction mixture was diluted with H₂O (40 mL) and extracted with EA (30 mL×2). The combined organic layers were washed with brine (50 mL), dried over Na₂SO₄, filtered and concentrated in vacuo to give a residue, which was purified (PM1) to afford compound 2.061 (200 mg, 242.79 μmol, 80.80% yield) as yellow oil.

LCMS (AM3): rt=1.157 min, (824.4 [M+H]⁺), 99.4% purity.

### EXAMPLE COMPOUNDS

The Examples are prepared according to the methods below using the preparations herein before. Wherein additional materials have been prepared, preparations are included for each Example. Alternatively, wherein commercially available materials are used, only the final steps are included, and no intermediate reference number is necessary.

### Reference Example 1

### 5-(2-(4-((3,5-difluoro-4-(trifluoromethoxy)benzyl)amino)butoxy)ethoxy)benzo[c][2,6]naphthyridine-8-carboxylic acid

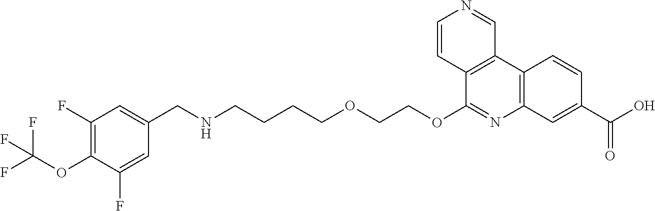 [SMILES: O=C(O)c1ccc2c(c1)nc(OCCOCCCCNCc1cc(F)c(OC(F)(F)F)c(F)c1)c1ccncc12]

A mixture of Intermediate Q (300 mg, 514.20 μmol), compound 1.507 (116.26 mg, 514.20 μmol) and DIPEA (199.36 mg, 1.54 mmol) in MeOH (5 mL) was stirred at 25° C. for 1 h, then NaB(OAc)₃H (544.90 mg, 2.57 mmol) was added. The mixture was stirred at 25° C. for 11 h. The mixture was concentrated in vacuo and the residue purified (PM12) to afford REFERENCE EXAMPLE 1 (99.09 mg, 175.23 mol, 34.1% yield) as a yellow solid.

LCMS (AM7): rt=0.769 min, (566.3 [M+H]⁺), 100% purity.

¹H NMR (400 MHz, MeOH-d₄) δ: 9.69 (s, 1H), 8.65 (d, J=5.6 Hz, 1H), 8.37 (d, J=8.4 Hz, 1H), 8.27 (d, J=1.6 Hz, 1H), 8.01 (dd, J=8.4, 1.6 Hz, 1H), 7.87 (d, J=5.2 Hz, 1H), 7.38 (d, J=8.4 Hz, 2H), 4.64 (t, J=4.8 Hz, 2H), 4.16 (s, 2H), 3.90 (t, J=4.8 Hz, 2H), 3.68 (t, J=6.0 Hz, 2H), 3.04 (t, J=7.6 Hz, 2H), 1.91-1.84 (quin, 2H), 1.76-1.69 (quin, 2H) ppm.

### Example 2

### isopropyl 5-(2-(4-((3,5-difluoro-4-(trifluoromethoxy)benzyl)amino)butoxy)ethoxy)benzo[c][2,6]naphthyridine-8-carboxylate

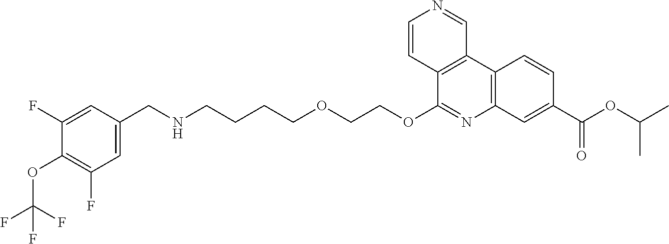 [SMILES: CC(C)OC(=O)c1ccc2c(c1)nc(OCCOCCCCNCc1cc(F)c(OC(F)(F)F)c(F)c1)c1ccncc12]

To a solution of compound 2.052 (100 mg, 141.31 μmol) in DCM (1.5 mL) was added TFA (1.23 g, 10.80 mmol). The reaction mixture was stirred at 20° C. for 0.5 hour. The reaction mixture was concentrated in vacuo to give the residue, which was purified (PM13) to afford EXAMPLE 2 (44.57 mg, 73.36 μmol, 51.91% yield) as a yellow solid.

LCMS (AM3): rt=0.911 min, (608.0 [M+H]⁺), 99.3% purity.

¹H NMR (400 MHz, METHANOL-d₄) δ: 10.00 (s, 1H), 8.86 (d, J=5.2 Hz, 1H), 8.74 (d, J=8.8 Hz, 1H), 8.43 (s, 1H), 8.19 (d, J=5.6 Hz, 1H), 8.15-8.10 (m, 1H), 7.29 (d, J=8.4 Hz, 2H), 5.34-5.27 (m, 1H), 4.84-4.81 (m, 2H), 4.12 (s, 2H), 4.02-3.95 (m, 2H), 3.73 -3.63 (m, 2H), 3.05 (t, J=8.0 Hz, 2H), 1.85-1.80 (m, 2H), 1.80-1.71 (m, 2H), 1.44 (d, J=6.4 Hz, 6H) ppm.

### Example 3

### 2-(dimethylamino)ethyl 5-(2-(4-((3,5-difluoro-4-(trifluoromethoxy)benzyl)amino)butoxy)ethoxy)benzo[c][2,6]naphthyridine-8-carboxylate

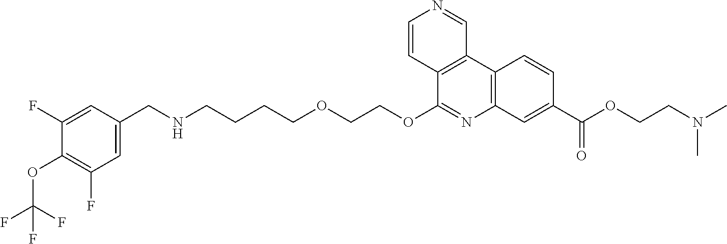 [SMILES: CN(C)CCOC(=O)c1ccc2c(c1)nc(OCCOCCCCNCc1cc(F)c(OC(F)(F)F)c(F)c1)c1ccncc12]

To a solution of compound 2.053 (100 mg, 135.74 μmol) in DCM (1 mL) was added TFA (770.00 mg, 6.75 mmol). The reaction mixture was stirred at 20° C. for 0.5 hour. The reaction mixture was concentrated in vacuo to give a residue, which was purified (PM30) and then re-purified by (PM14) to afford EXAMPLE 3 (32.41 mg, 50.91 μmol, 57.55% yield) as yellow oil.

LCMS (AM7): rt=1.191 min, (637.2 [M+H]⁺), 97.4% purity.

¹H NMR (400 MHz, METHANOL-d₄) δ10.04 (s, 1H), 8.87 (d, J=5.6 Hz, 1H), 8.79 (d, J=8.4 Hz, 1H), 8.55 (d, J=1.6 Hz, 1H), 8.24 (dd, J=5.6, 0.8 Hz, 1H), 8.21 (dd, J=8.8, 2.0 Hz, 1H), 7.13 (s, 1H), 7.10 (s, 1H), 4.84-4.82 (m, 2H), 4.54 (t, J=5.6 Hz, 2H), 4.0-3.96 (m, 2H), 3.68 (s, 2H), 3.64 (t, J=5.6 Hz, 2H), 2.84 (t, J=5.6 Hz, 2H), 2.57 (t, J=7.2 Hz, 2H), 2.40 (s, 6H), 1.68-1.58 (m, 4H) ppm.

### Example 4

### methyl 5-(2-(4-((3,5-difluoro-4-(trifluoromethoxy)benzyl)amino)butoxy)ethoxy)benzo[c][2,6]naphthyridine-8-carboxylate

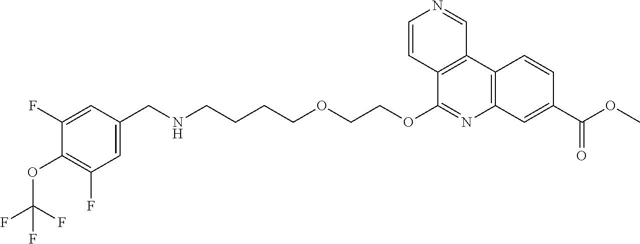 [SMILES: COC(=O)c1ccc2c(c1)nc(OCCOCCCCNCc1cc(F)c(OC(F)(F)F)c(F)c1)c1ccncc12]

To a solution of compound 2.054 (100 mg, 147.14 mol) in DCM (2 mL) was added TFA (1.54 g, 13.51 mmol). The reaction mixture was stirred at 20° C. for 0.5 hour. The reaction mixture was concentrated in vacuo to give the residue, which was purified (PM31) and then re-purified (PM14) to afford EXAMPLE 4 (38.15 mg, 65.83 μmol, 64.84% yield) as a white solid.

LCMS (AM3): rt=0.876 min, (580.0 [M+H]⁺), 100% purity.

¹H NMR (400 MHz, METHANOL-d₄) δ 9.87 (s, 1H), 8.79 (d, J=5.6 Hz, 1H), 8.60 (d, J=8.4 Hz, 1H), 8.29 (d, J=1.6 Hz, 1H), 8.11 (d, J=5.2 Hz, 1H), 8.02 (dd, J=8.4, 1.6 Hz, 1H), 7.11 (d, J=8.8 Hz, 2H), 4.78-4.74 (m, 2H), 3.98 (s, 3H), 3.98-3.94 (m, 2H), 3.68 (s, 2H), 3.64 (t, J=5.6 Hz, 2H), 2.57 (t, J=7.2 Hz, 2H), 1.70-1.59 (m, 4H) ppm.

### Example 5

### (isobutyryloxy)methyl 5-(2-(4-((3,5-difluoro-4-(trifluoromethoxy)benzyl)amino)butoxy)ethoxy)benzo[c][2,6]naphthyridine-8-carboxylate

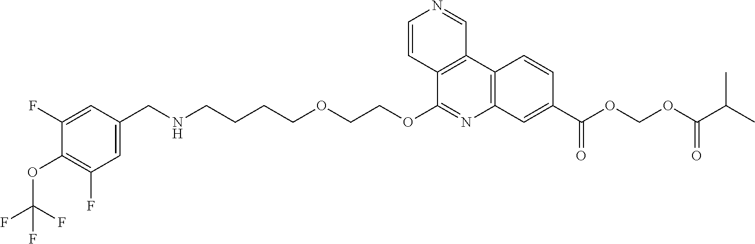 [SMILES: CC(C)C(=O)OCOC(=O)c1ccc2c(c1)nc(OCCOCCCCNCc1cc(F)c(OC(F)(F)F)c(F)c1)c1ccncc12]

To a solution of compound 2.055 (170 mg, 222.01 μmol) in DCM (3 mL) was added TFA (231.00 mg, 2.03 mmol), the reaction mixture was stirred at 10° C. for 12 hours. Additional TFA (231.00 mg, 2.03 mmol) was added and the reaction was stirred at 10° C. for 12 hours. The reaction mixture was adjusted to pH=8 with DIPEA and concentrated in vacuo to give a residue, which was purified (PM15) to afford a crude product which was re-purified (PM16) to afford EXAMPLE 5 (32.34 mg, 48.59 μmol, 62.71% yield) as a yellow gum.

LCMS (AM3): rt=0.910 min, (666.3 [M+H]⁺), 100% purity.

¹H NMR (400 MHz, DMSO-d₆) δ 10.22 (s, 1H), 9.02-8.97 (m, 2H), 8.37 (d, J=1.6 Hz, 1H), 8.13-8.08 (m, 2H), 7.39 (d, J=9.6 Hz, 2H), 6.03 (s, 2H), 4.78-4.73 (m, 2H), 3.92-3.87 (m, 2H), 3.83 (s, 2H), 3.54 (t, J=5.6 Hz, 2H), 2.70-2.61 (m, 3H), 1.60-1.52 (s, 4H), 1.12 (d, J=7.2 Hz, 6H) ppm.

### Example 6

### (pivaloyloxy)methyl 5-(2-(4-((3,5-difluoro-4-(trifluoromethoxy)benzyl)amino)butoxy)ethoxy)benzo[c][2,6]naphthyridine-8-carboxylate

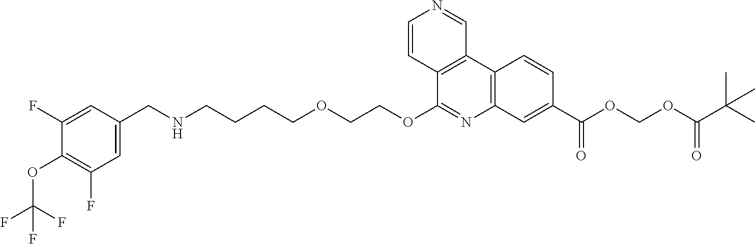 [SMILES: CC(C)(C)C(=O)OCOC(=O)c1ccc2c(c1)nc(OCCOCCCCNCc1cc(F)c(OC(F)(F)F)c(F)c1)c1ccncc12]

To a solution of compound 2.056 (70 mg, 89.77 mol) in DCM (3 mL) was added TFA (231.00 mg, 2.03 mmol) and the reaction mixture stirred at 10° C. for 12 hours. Additional TFA (231.00 mg, 2.03 mmol) was added and the reaction was stirred at 10° C. for 12 hours. The reaction mixture was adjusted to pH=8 with DIPEA. Then the reaction mixture was concentrated in vacuo to give a residue, which was purified (PM17) to afford EXAMPLE 6 (30.35 mg, 44.66 μmol, 49.74% yield) as a yellow gum.

LCMS (AM3): rt=0.942 min, (680.3 [M+H]⁺), 99.2% purity.

¹H NMR (400 MHz, DMSO-d₆) δ 10.22 (s, 1H), 9.02-8.97 (m, 2H), 8.37 (d, J=1.6 Hz, 1H), 8.13-8.08 (m, 2H), 7.31 (d, J=9.6 Hz, 2H), 6.04 (s, 2H), 4.78-4.74 (m, 2H), 3.92-3.87 (m, 2H), 3.73 (s, 2H), 3.54 (t, J=6.0 Hz, 2H), 2.52 (s, 2H), 1.60-1.54 (m, 2H), 1.52-1.46 (m, 2H), 1.18 (s, 9H) ppm.

### Example 7

### (5-methyl-2-oxo-1,3-dioxol-4-yl)methyl 5-(2-(4-((3,5-difluoro-4-(trifluoromethoxy)benzyl)amino)butoxy)ethoxy)benzo[c][2,6]naphthyridine-8-carboxylate

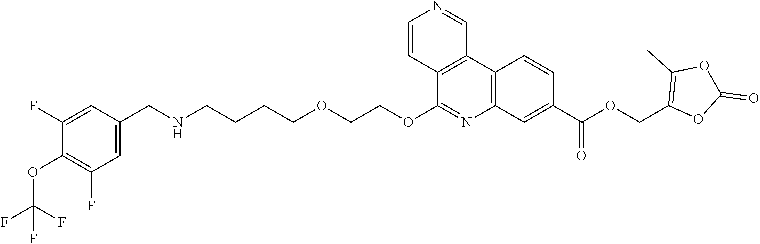 [SMILES: Cc1oc(=O)oc1COC(=O)c1ccc2c(c1)nc(OCCOCCCCNCc1cc(F)c(OC(F)(F)F)c(F)c1)c1ccncc12]

To a solution of compound 2.057 (220 mg, 282.89 μmol) in DCM (2 mL) was added TFA (308.00 mg, 2.70 mmol) slowly. The reaction mixture was stirred at 10° C. for 14 hours. TFA (308.00 mg, 2.70 mmol) in DCM (2 mL) was added slowly and then the reaction was stirred at 10° C. for another 2 hours. The reaction mixture was adjusted to pH=10 with DIPEA at 0° C. The mixture was concentrated in vacuo to give a residue, which was purified (PM18) and then re-purified (PM19) to afford EXAMPLE 7 (34.91 mg, 51.19 mol, 18.09% yield) as a yellow solid.

LCMS (AM3): rt=0.896 min, (678.3 [M+H]⁺), 99.4% purity.

¹H NMR (400 MHz, DMSO-d₆) δ 10.18 (s, 1H), 8.98-8.92 (m, 2H), 8.34 (s, 1H), 8.10-8.05 (m, 2H), 7.27 (d, J=10.4 Hz, 2H), 5.30 (s, 2H), 4.75-4.69 (m, 2H), 3.90-3.85 (m, 2H), 3.64 (s, 2H), 3.52 (t, J=6.0 Hz, 2H), 2.43 (t, J=6.4 Hz, 2H), 2.26 (s, 3H), 1.60-1.53 (m, 2H), 1.49-1.42 (m, 2H) ppm.

### Example 8

### ethyl 5-(2-(4-((3,5-difluoro-4-(trifluoromethoxy)benzyl)amino)butoxy)ethoxy)benzo[c][2,6]naphthyridine-8-carboxylate

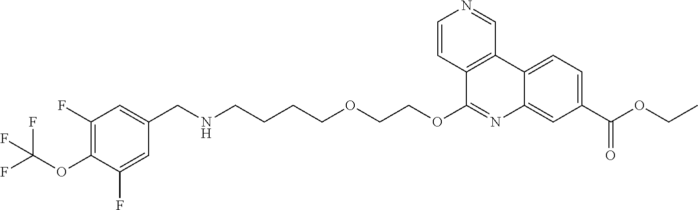 [SMILES: CCOC(=O)c1ccc2c(c1)nc(OCCOCCCCNCc1cc(F)c(OC(F)(F)F)c(F)c1)c1ccncc12]

A mixture of compound 2.046 (200 mg, 288.33 μmol) and TFA (67.53 mmol, 5 mL) in DCM (5 mL) was stirred at room temperature for 1 hour. The reaction mixture was concentrated in vacuo to give a residue, which was purified (PM20) then repurified by (PM21) to afford EXAMPLE 8 (52.58 mg, 53.71% yield, 100% purity) as a white solid.

LCMS (AM3): rt=0.910 min, (594.3 [M+H]⁺), 100% purity.

¹H NMR (400 MHz, METHANOL-d₄) δ 9.95 (s, 1H), 8.83 (d, J=5.6 Hz, 1H), 8.69 (d, J=8.4 Hz, 1H), 8.39 (d, J=1.6 Hz, 1H), 8.17 (d, J=5.6 Hz, 1H), 8.10 (dd, J=8.4, 1.6 Hz, 1H), 7.11 (d, J=8.8 Hz, 2H), 4.82-4.78 (m, 2H), 4.45 (q, J=7.2 Hz, 2H), 4.00-3.95 (m, 2H), 3.68 (s, 2H), 3.64 (t, J=6.0 Hz, 2H), 2.57 (t, J=6.8 Hz, 2H), 1.70-1.58 (m, 4H), 1.46 (t, J=7.2 Hz, 3H) ppm.

### Example 9

### tert-butyl 5-(2-(4-((3,5-difluoro-4-(trifluoromethoxy)benzyl)amino)butoxy)ethoxy)benzo[c][2,6]naphthyridine-8-carboxylate

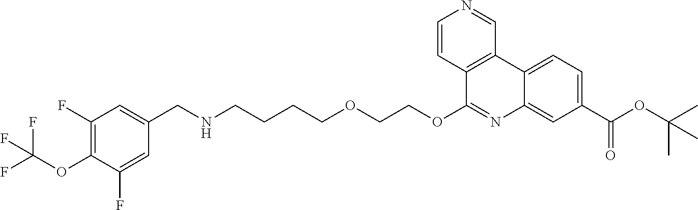 [SMILES: CC(C)(C)OC(=O)c1ccc2c(c1)nc(OCCOCCCCNCc1cc(F)c(OC(F)(F)F)c(F)c1)c1ccncc12]

A mixture of compound 2.047 (200 mg, 277.12 μmol) and TFA (1.35 mmol, 0.1 mL) in DCM (10 mL) was stirred at 16° C. for 16 hours. Additional TFA (2.70 mmol, 0.2 mL) was added and the reaction mixture was stirred for 4 hours. The reaction mixture was adjusted to pH=8 with DIPEA and concentrated in vacuo to give a residue, which was purified (PM22) to afford EXAMPLE 9 (33.60 mg, 19.14% yield) as a brown solid.

LCMS (AM3): rt=0.936 min, (622.4 [M+H]⁺), 98.16% purity.

¹H NMR (400 MHz, DMSO-d₆) δ 10.03 (s, 1H), 8.88 (s, 1H), 8.72 (d, J=8.4 Hz, 1H), 8.38 (d, J=1.6 Hz, 1H), 8.20 (d, J=3.6 Hz, 1H), 8.09 (dd, J=8.4, 1.6 Hz, 1H), 7.27 (d, J=8.4 Hz, 2H), 4.82 (dd, J=5.2, 4.0 Hz, 2H), 4.07 (s, 2H), 4.02-3.98 (m, 2H), 3.68 (t, J=5.6 Hz, 2H), 2.99-2.98 (m, 2H), 1.82-1.70 (m, 4H), 1.67 (s, 9H) ppm.

### Example 10

### acetoxymethyl 5-(2-(4-((3,5-difluoro-4-(trifluoromethoxy)benzyl)amino)butoxy)ethoxy)benzo[c][2,6]naphthyridine-8-carboxylate

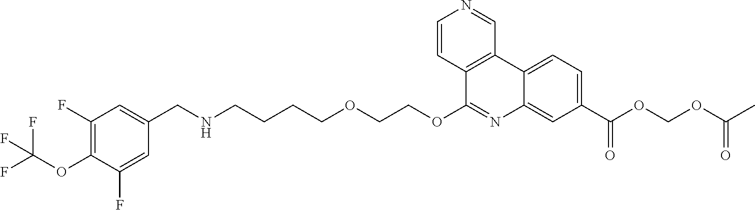 [SMILES: CC(=O)OCOC(=O)c1ccc2c(c1)nc(OCCOCCCCNCc1cc(F)c(OC(F)(F)F)c(F)c1)c1ccncc12]

A mixture of compound 2.048 (120 mg, 162.68 μmol) and TFA (2.70 mmol, 0.2 mL) in DCM (2 mL) was stirred at room temperature for 20 hours. The reaction mixture was concentrated in vacuo to give a residue, which was purified (PM23) to afford EXAMPLE 10 (26.00 mg, 24.53% yield) as a yellow gum.

LCMS (AM3): rt=0.889 min, (638.3 [M+H]⁺), 100.00% purity.

¹H NMR (400 MHz, METHANOL-d4) δ 9.81 (s, 1H), 8.77 (d, J=5.6 Hz, 1H), 8.54 (d, J=8.4 Hz, 1H), 8.21 (s, 1H), 8.05 (d, J=5.6 Hz, 1H), 7.97 (dd, J=8.4, 1.6 Hz, 1H), 7.11 (d, J=8.8 Hz, 2H), 6.04 (s, 2H), 4.75-4.70 (m, 2H), 3.99-3.92 (m, 2H), 3.70 (s, 2H), 3.65 (t, J=6.0 Hz, 2H), 2.59 (t, J=6.8 Hz, 2H), 2.18 (s, 3H), 1.72-1.60 (m, 4H) ppm.

### Example 11

### ((methoxycarbonyl)oxy)methyl 5-(2-(4-((3,5-difluoro-4-(trifluoromethoxy)benzyl)amino)butoxy)ethoxy)benzo[c][2,6]naphthyridine-8-carboxylate

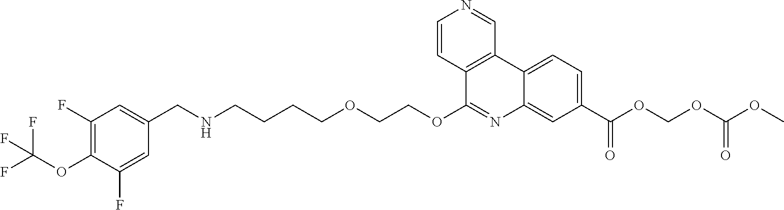 [SMILES: COC(=O)OCOC(=O)c1ccc2c(c1)nc(OCCOCCCCNCc1cc(F)c(OC(F)(F)F)c(F)c1)c1ccncc12]

A mixture of compound 2.049 (170 mg, 225.56 μmol) and TFA (6.75 mmol, 0.5 mL) in DCM (5 mL) was stirred at room temperature for 0.5 hour. The reaction mixture was adjusted to pH=8 with DIPEA and concentrated in vacuo to give a residue, which was purified (PM23) to afford EXAMPLE 11 (19.94 mg, 66.20% yield) as a white solid.

LCMS (AM3): rt=0.902 min, (652.3 [M+H]⁺), 100% purity.

¹H NMR (400 MHz, METHANOL-d₄) δ 10.00 (s, 1H), 8.87 (d, J=5.6 Hz, 1H), 8.77 (d, J=8.4 Hz, 1H), 8.47 (d, J=1.6 Hz, 1H), 8.26-8.19 (m, 1H), 8.15 (dd, J=8.4, 1.6 Hz, 1H), 7.12 (d, J=8.8 Hz, 2H), 6.07 (s, 2H), 4.84-4.81 (m, 2H), 3.99 (dd, J=5.2, 4.0 Hz, 2H), 3.86 (s, 3H), 3.70 (s, 2H), 3.65 (t, J=6.0 Hz, 1H), 2.58 (t, J=6.8 Hz, 2H), 1.72-1.58 (m, 4H) ppm.

### Example 12

### (propionyloxy)methyl 5-(2-(4-((3,5-difluoro-4-(trifluoromethoxy)benzyl)amino)butoxy)ethoxy)benzo[c][2,6]naphthyridine-8-carboxylate

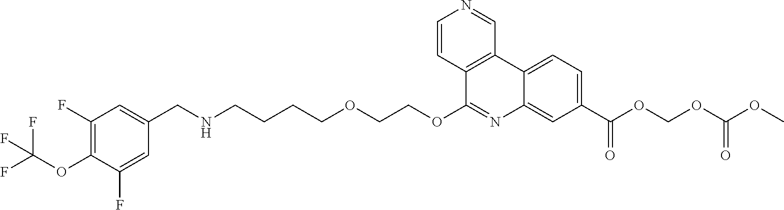 [SMILES: COC(=O)OCOC(=O)c1ccc2c(c1)nc(OCCOCCCCNCc1cc(F)c(OC(F)(F)F)c(F)c1)c1ccncc12]

A mixture of compound 2.050 (140 mg, 186.25 μmol) and TFA (5.40 mmol, 0.4 mL) in DCM (4 mL) was stirred at room temperature for 2 hours. The reaction mixture was adjusted to pH=8 with DIPEA and concentrated in vacuo to give a residue, which was purified (PM24) and then repurified (PM25) to afford EXAMPLE 12 (18.04 mg, 59.83% yield) as yellow oil.

LCMS (AM3): rt=0.902 min, (652.3 [M+H]⁺), 100% purity.

¹H NMR (400 MHz, METHANOL-d₄) δ 10.00 (s, 1H), 8.87 (d, J=5.6 Hz, 1H), 8.77 (d, J=8.4 Hz, 1H), 8.46 (d, J=1.6 Hz, 1H), 8.22 (dd, J=5.6, 0.8 Hz, 1H), 8.14 (dd, J=8.4, 1.6 Hz, 1H), 7.12 (d, J=8.8 Hz, 2H), 6.07 (s, 2H), 4.83 (dd, J=3.6, 2.4 Hz, 2H), 3.98 (dd, J=5.2, 4.0 Hz, 2H), 3.70 (s, 2H), 3.64-3.64 (m, 1H), 3.65 (t, J=6.0 Hz, 1H), 2.58 (t, J=6.8 Hz, 2H), 2.47 (q, J=7.6 Hz, 2H), 1.68-1.58 (m, 4H), 1.17 (t, J=7.6 Hz, 3H) ppm.

### Example 13

### ((ethoxycarbonyl)oxy)methyl 5-(2-(4-((3,5-difluoro-4-(trifluoromethoxy)benzyl)amino)butoxy)ethoxy)benzo[c][2,6]naphthyridine-8-carboxylate

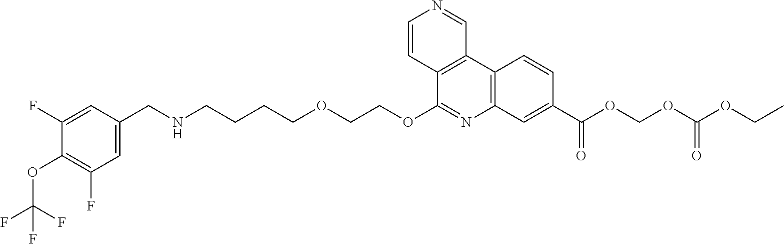 [SMILES: CCOC(=O)OCOC(=O)c1ccc2c(c1)nc(OCCOCCCCNCc1cc(F)c(OC(F)(F)F)c(F)c1)c1ccncc12]

To a solution of compound 2.058 (200 mg, 260.52 μmol) in DCM (4 mL) was added TFA (616.00 mg, 5.40 mmol) slowly at 10° C. The reaction mixture was stirred at 10° C. for 2 hours. The reaction mixture was adjusted to pH=8 with DIPEA at 0° C. and then concentrated in vacuo to give a residue, which was purified (PM26) to afford a crude product, which was re-purified (PM27) to afford EXAMPLE 13 (17.12 mg, 25.65 μmol, 50.57% yield) as a yellow solid.

LCMS (AM3): rt=0.896 min, (668.3 [M+H]⁺), 100% purity.

¹H NMR (400 MHz, DMSO-d6) δ 10.19 (s, 1H), 9.00-8.95 (m, 2H), 8.36 (d, J=1.6 Hz, 1H), 8.11-8.07 (m, 2H), 7.32 (d, J=9.6 Hz, 2H), 6.03 (s, 2H), 4.77-4.72 (m, 2H), 4.26-4.19 (m, 2H), 3.91-3.86 (m, 2H), 3.71 (s, 2H), 3.53 (t, J=6.0 Hz, 2H), 2.48-2.42 (m, 2H), 1.62-1.54 (m, 2H), 1.53-1.46 (m, 2H), 1.24 (t, J=7.2 Hz, 3H) ppm.

### Example 14

### ((isopropoxycarbonyl)oxy)methyl 5-(2-(4-((3,5-difluoro-4-(trifluoromethoxy)benzyl)amino)butoxy)ethoxy)benzo[c][2,6]naphthyridine-8-carboxylate

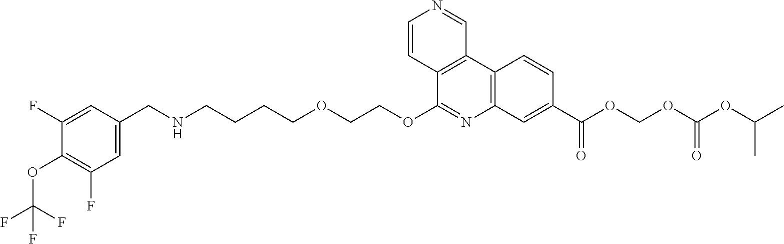 [SMILES: CC(C)OC(=O)OCOC(=O)c1ccc2c(c1)nc(OCCOCCCCNCc1cc(F)c(OC(F)(F)F)c(F)c1)c1ccncc12]

To a solution of compound 2.059 (160 mg, 204.68 mol) in DCM (4 mL) was added TFA (616.00 mg, 5.40 mmol) slowly at 10° C. The reaction mixture was stirred at 10° C. for 1 hour. The reaction mixture was adjusted to pH=8 with DIPEA (0.8 mL) slowly at 0° C. and then concentrated in vacuo to give a residue, which was purified (PM15) to afford a crude product. which was re-purified (PM28) to afford EXAMPLE 14 (24.18 mg, 35.48 mol, 50.32% yield) as yellow oil.

LCMS (AM7): rt=1.206 min, (682.2 [M+H]⁺), 100% purity.

¹H NMR (400 MHz, DMSO-d₆) δ 10.22 (s, 1H), 9.03-8.97 (m, 2H), 8.39 (d, J=2.0 Hz, 1H), 8.14-8.09 (m, 2H), 7.27 (d, J=9.6 Hz, 2H), 6.02 (s, 2H), 4.89-4.82 (m, 1H), 4.78-4.74 (m, 2H), 3.91-3.87 (m, 2H), 3.63 (s, 2H), 3.53 (t, J=6.4 Hz, 2H), 2.43 (s, 2H), 1.59-1.53 (m, 2H), 1.49-1.43 (m, 2H), 1.26 (d, J=6.4 Hz, 6H) ppm.

### Example 15

### ((tert-butoxycarbonyl)oxy)methyl 5-(2-(4-((3,5-difluoro-4-(trifluoromethoxy)benzyl)amino)butoxy)ethoxy)benzo[c][2,6]naphthyridine-8-carboxylate

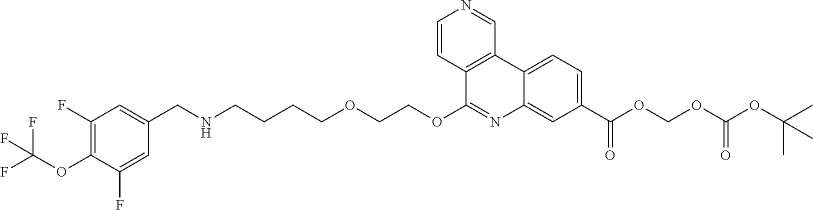 [SMILES: CC(C)(C)OC(=O)OCOC(=O)c1ccc2c(c1)nc(OCCOCCCCNCc1cc(F)c(OC(F)(F)F)c(F)c1)c1ccncc12]

A mixture of compound 2.051 (300 mg, 377.01 μmol) and TFA (8.10 mmol, 0.6 mL) in DCM (12 mL) was stirred at room temperature for 3 hours. The reaction mixture was concentrated in vacuo to give a residue, which was purified (PM29) and then repurified by prep-TLC (DCM:MeOH=20:1) to afford EXAMPLE 15 (38.33 mg, 14.62% yield) as a yellow solid.

LCMS (AM3): rt=0.936 min, (696.4 [M+H]⁺), 100% purity.

¹H NMR (400 MHz, METHANOL-d4) δ 10.03 (s, 1H), 8.88 (d, J=5.6 Hz, 1H), 8.79 (d, J=8.4 Hz, 1H), 8.47 (d, J=1.6 Hz, 1H), 8.21 (d, J=5.6 Hz, 1H), 8.16 (dd, J=8.4, 1.6 Hz, 1H), 7.33 (d, J=8.4 Hz, 2H), 6.01 (s, 2H), 4.84 (t, J=4.8 Hz, 2H), 4.18 (s, 2H), 4.01 (t, J=4.4 Hz, 2H), 3.70 (t, J=6.0 Hz, 1H), 3.65 (t, J=6.0 Hz, 1H), 3.01 (t, J=6.8 Hz, 2H), 1.92-1.81 (m, 2H), 1.81-1.70 (m, 2H), 1.52 (s, 9H) ppm.

### Example 16

### ((((tetrahydro-2H-pyran-4-yl)oxy)carbonyl)oxy)methyl 5-(2-(4-((3,5-difluoro-4-(trifluoromethoxy)benzyl)amino)butoxy)ethoxy)benzo[c][2,6]naphthyridine-8-carboxylate

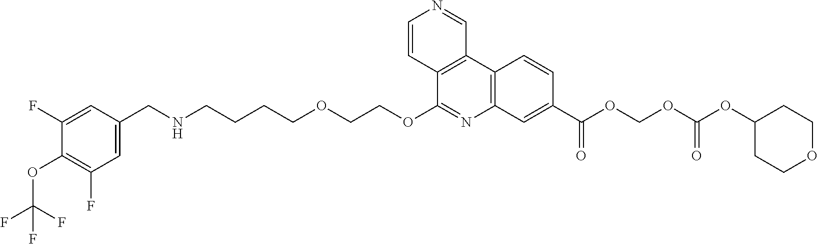 [SMILES: O=C(OCOC(=O)c1ccc2c(c1)nc(OCCOCCCCNCc1cc(F)c(OC(F)(F)F)c(F)c1)c1ccncc12)OC1CCOCC1]

To a solution of compound 2.061 (200 mg, 242.79 mol) in DCM (4 mL) was added TFA (616.00 mg, 5.40 mmol) slowly at 0° C. The reaction mixture was stirred at 10° C. for 1.5 hours. The reaction mixture was adjusted to pH=8 with DIPEA at 0° C. and concentrated in vacuo to give a residue, which was purified (PM20) to give a crude product which was re-purified (PM25) to afford EXAMPLE 16 (29.11 mg, 40.23 μmol, 52.86% yield) as an off-white gum.

LCMS (AM7): rt=1.160 min, (724.2 [M+H]⁺), 100% purity.

¹H NMR (400 MHz, DMSO-d₆) δ 10.21 (s, 1H), 9.01-8.96 (m, 2H), 8.38 (d, J=1.6 Hz, 1H), 8.13-8.08 (m, 2H), 7.27 (d, J=9.2 Hz, 2H), 6.04 (s, 2H), 4.89-4.82 (m, 1H), 4.77-4.73 (m, 2H), 3.90-3.87 (m, 2H), 3.82-3.75 (m, 2H), 3.64 (s, 2H), 3.53 (t, J=6.4 Hz, 2H), 3.47-3.42 (m, 2H), 2.43 (s, 2H), 1.96-1.90 (m, 2H), 1.61-1.44 (m, 6H) ppm.

### (A) Stability in Aqueous Media

### Stock Solution Preparation:

### Examples

10 μL of a 10 mM stock solution of the Examples were dissolved in 490 μL DMSO to afford 200 μL working solution SGF (pH 1.2):

Dissolve 0.04 g NaCl and 0.064 g pepsin in 0.14 mL HCl and add sufficient water to 20 mL volume. The pH of the test solution was 1.20±0.05.

FaSSIF (pH 6.5):

0.056% (w/v) lecithin, 0.161% (w/v) sodium taurocholate, 0.39% (w/v) monobasic potassium phosphate, 0.77% (w/v) potassium chloride, deionized H₂O. The pH of the test solution was 6.5±0.05.

### PBS (pH 7.4):

30 mL of 10×PBS (pH7.4) was added into a 50 mL flask and diluted with water. The solution was mixed well and adjusted to pH 7.4 with 1 N HCl.

### Assay Procedure:

2 μL of the 200 μL working solution of the Examples was added into 96-deep-well plates for TO, T60, T120, T360 and T1440 timepoints. Duplicates were prepared.

Stability studies were commenced by the addition of 198 μL of the required media (e.g. Simulated Gastric Fluid pH 1.2 [SGF], Simulated Fasted State Intestinal Fluid pH 6.5 [FaSSIF]) and Phosphate Buffered Saline pH 7.4 [PBS]) to each well except TO. The samples were incubated at 37° C. at 600 rpm for the appointed time. The samples at the corresponding timepoint were removed and immediately mixed with 400 μL of cold acetonitrile containing 200 ng/mL tolbutamide and labetalol (internal standard). of cold acetonitrile containing 200 ng/mL tolbutamide and labetalol. The samples were centrifuged at 4000 rpm at 4° C. for 20 minutes. 60 μL of the supernatant was removed and mixed with 180 μL purified water and thoroughly mixed and analysed by LCMS (Shimadzu LC 30-AD, ACQUITY UPLC BEH Phenyl 1.7 um 2.1*50 mm Part No. 186002884, with mobile phase A: 0.1% formic acid in water, mobile phase B: 0.1% formic acid in acetonitrile). Formation of parent was also monitored as a metabolite.

Samples were analysed by Mass Spectrometry QTRAP 6500+.

The stability of the Examples in various aqueous media are shown below in Table 1:

TABLE 1
Stability of Examples in FaSSIF and SGF at 1, 6 and
24 hour timepoints. % remaining at chosen timepoint.

| | FaSSIF | | | SGF | | | PBS | | |
|---|---|---|---|---|---|---|---|---|---|
| | (pH 6.5) | | | (pH 1.2) | | | (pH 7.4) | | |
| | % remaining | | | % remaining | | | % remaining | | |
| Ex. No | 1 h | 6 h | 24 h | 1 h | 6 h | 24 h | 1 h | 6 h | 24 h |
| 2 | 95.4 | 80.5 | 87.2 | 97.1 | 57.9 | 19.6 | 74.5 | 31.0 | 11.5 |
| 3 | 102.2 | 101.8 | 98.6 | 90.6 | 76.1 | 33.5 | 71.8 | 17.8 | 1.3 |
| 4 | 90.0 | 86.6 | 93.3 | 93.7 | 63.7 | 16.5 | 70.3 | 22.4 | 7.9 |
| 5 | 100.2 | 97.5 | 98.8 | 87.0 | 55.5 | 13.0 | 76.2 | 7.9 | 2.4 |
| 6 | 112.8 | 97.0 | 109.4 | 88.8 | 59.8 | 20.0 | 85.2 | 27.7 | 5.1 |
| 7 | 101.0 | 93.5 | 89.1 | 98.9 | 67.2 | 26.9 | 78.9 | 16.7 | 0.7 |
| 8 | 98.5 | 102.6 | 100.4 | 108.2 | 63.8 | 23.3 | 63.2 | 25.3 | 7.6 |
| 9 | 92.1 | 98.3 | 96.1 | 102.1 | 57.9 | 19.6 | 80.9 | 27.9 | 10.0 |
| 10 | 101.7 | 97.1 | 104.4 | 101.5 | 55.9 | 10.1 | 78.8 | 7.1 | 1.1 |
| 11 | 96.4 | 97.5 | 105.9 | 100.2 | 66.3 | 21.5 | 81.2 | 29.3 | 19.0 |
| 12 | 94.9 | 96.5 | 99.8 | 101.3 | 49.5 | 10.1 | 76.4 | 6.3 | 1.1 |
| 13 | 106.5 | 99.8 | 96.8 | 106.8 | 63.6 | 21.5 | 84.3 | 17.5 | 20.5 |
| 14 | 98.2 | 93.6 | 102.8 | 105.1 | 63.8 | 23.2 | 91.7 | 34.3 | 4.4 |
| 15 | 99.0 | 97.9 | 98.6 | 47.5 | 3.8 | 1.8 | 100.3 | 49.7 | 7.9 |
| 16 | 95.5 | 104.4 | 95.7 | 95.4 | 63.0 | 19.4 | 93.8 | 37.9 | 7.1 |

### (B) Solubility

### Protocol for Determining Solubility in Simulated Fluids:

A known quantity of the Examples and control compounds were weighed into a suitable vessel (whatman miniuniprep vials) and a known volume of the required media was added (e.g. Simulated Gastric Fluid pH 1.2 [SGF], Simulated Fasted State Intestinal Fluid pH 6.5 [FaSSIF] or Phosphate Buffered Saline pH 7.4 [PBS]). The samples were subjected to vortex mixing for 2 minutes followed by agitation on a shaker for 24 hours at room temperature at 800 rpm. The samples were then centrifuged for 20 minutes at 4000 rpm, the miniuniprep vials compressed and the filtrates analysed by HPLC (Waters Xbridge C18 4.6×100 mm, mobile phase A: 0.1% TFA and 5 mM NH40Ac in wate/acetonitrile (v:v 95:5), mobile phase B: 0.1% TFA and 5 mN NH40Ac in water/acetonitrile (v:v 5:95). The parent compound (Reference Example 1) is included as a comparison. The results indicate that, overall, the examples are more soluble than the parent compound.

The solubility of the Examples in various aqueous media are shown below in Table 2:

TABLE 2
Solubility of Examples in FaSSIF and SGF at 24 hour timepoint.

| Example No | Media | Kinetic solubility (μg/mL) |
|---|---|---|
| 2 | FaSSIF | ND |
| | SGF | ND |
| 3 | FaSSIF | ND |
| | SGF | ND |
| 4 | FaSSIF | ND |
| | SGF | ND |
| 5 | FaSSIF | ND |
| | SGF | ND |
| 6 | FaSSIF | ND |
| | SGF | ND |
| 7 | FaSSIF | 76.1 |
| | SGF | 18.5 |
| 8 | FaSSIF | 82.8 |
| | SGF | 17.8 |
| 9 | FaSSIF | 88.3 |
| | SGF | 16.1 |
| 10 | FaSSIF | 91.3 |
| | SGF | 20.9 |
| 11 | FaSSIF | ND |
| | SGF | ND |
| 12 | FaSSIF | 97.5 |
| | SGF | 7.4 |
| 13 | FaSSIF | 98.0 |
| | SGF | 17.8 |
| 14 | FaSSIF | 100.3 |
| | SGF | 15.0 |
| 15 | FaSSIF | ND |
| | SGF | ND |
| 16 | FaSSIF | 99.4 |
| | SGF | 39.4 |
| 1 | FaSSIF | ND |
| | SGF | ND |

TABLE 3
comparative solubility data for Reference Example 1

| solubility | | initial | solubility |
|---|---|---|---|
| type | medium | pH | (μg/mL) |
| thermodynamic | phosphate buffer | 7.4 | <0.9 |
| | | 1.0 | 256 |
| | | 10.0 | <0.9 |
| | FaSSIF | 6.5 | 1.6 |
| | FeSSIF | 5.0 | 10.7 |
| kinetic | phosphate buffer | 7.4 | 31 |

### (C) Intestinal Microsome Stability

### Stock Solution Preparation:

### EXAMPLES

5 μL of a 10 mM stock solution of the Examples were dissolved in 495 μL acetonitrile to afford 500 μL working solution.

### Nadph Cofactor:

The appropriate amount of β-Nicotinamide adenine dinucleotide phosphate reduced form, tetrasodium salt; NADPH-4Na (NADPH) powder was weighed and diluted into a 10 mM MgCl₂ solution (working solution concentration: 10 unit/mL; final concentration in reaction system: 1 unit/mL).

### Intestinal Microsomes:

The appropriate concentrations of rat intestinal microsome (Xenotech, cat. No R1000.1) working solutions were prepared in 100 mM potassium phosphate buffer.

### Stop Solution:

Cold (4° C.) acetonitrile (ACN) containing 200 ng/mL tolbutamide and 200 ng/mL labetalol as internal standards (IS).

### Assay Procedure:

Intestinal microsomes were diluted to 0.337 mg/mL in 100 mM potassium phosphate buffer and 445 μL transferred into pre-warmed T60 and NCF60 μlates (Incubation plates). Incubation plates were pre-incubated for 10 minutes at 37° C. with constant shaking. 54 μL was transferred from Incubation plates into a blank plate and then 6 μL NADPH cofactor and 180 μL quenching solution was added to the Incubation plates. 5 μL Example working solution (100 μM) was also added to the Incubation plates and mixed thoroughly.

The T60 Incubation plate was mixed thoroughly and for the 0-min timepoint, 54 μL was transferred into a Quenching plate (containing 180 μL quenching solution and 6 μL NADPH cofactor). 44 μL NADPH cofactor was then added to the T60 Incubation plate and incubated at 37° C. for 60 min while shaking. At 5, 15, 30, 45, and 60 min, 60 μL samples were transferred from T60 Incubation plate into a Quenching plate (containing 180 μl quenching solution). For the NCF60 Incubation plate, 50 μL of buffer was added and mixed thoroughly before incubation at 37° C. for 60 min while shaking, at the 60-min time point 60 μL was transferred to the Quenching plate (containing 180 μl quenching solution). All sampling plates were shaken for 10 min, then centrifuged at 4000 rpm for 20 minutes at 4° C. 80 μL supernatant was transferred into 240 μL HPLC water, and mixed for 10 min. Each bioanalysis plate was sealed and shaken for 10 minutes prior to LC-MS/MS analysis (Shimadzu LC 30-AD, ACQUITY UPLC BEH C18 1.7 um 2.1*50 mm Column, Part No. 186002350, with mobile phase A: 0.1% formic acid in water, mobile phase B: 0.1% formic acid in acetonitrile).

The stability of the Examples in rat intestinal microsomes is shown below in Table 3:

TABLE 3
Stability of Examples in rat intestinal microsomes.

| Example No | CI_{int(mic)} (μl/min/mg) | T_{1/2} (min) |
|---|---|---|
| 2 | ND | ND |
| 3 | ND | ND |
| 4 | ND | ND |
| 5 | ND | ND |
| 6 | ND | ND |
| 7 | ND | ND |
| 8 | 37.6 | 61.4 |
| 9 | 78.9 | 29.3 |
| 10 | 61.8 | 37.4 |
| 11 | ND | ND |
| 12 | 90.8 | 25.4 |
| 13 | 126.2 | 18.3 |
| 14 | 1604.8 | 1.4 |
| 15 | ND | ND |
| 16 | ND | ND |
| 1 | 17.2 | 134.6 |

### (D) Stability in Rat Plasma

### Stock Solution Preparation:

### Examples

5 μL of a 10 mM stock solution of the Examples were dissolved in 495 μL DMSO to afford 500 μL working solution Assay procedure:

Pooled frozen male SD rat plasma was thawed at 37° C. and centrifuged at 4000 rpm for 5 mins.

98 μL plasma was added to each well of a 96-well reaction plate, using one plate per timepoint (Blank, TO, T10, T30, T60, and T120). 2 μL of working solution was added to each well of the reaction plates except Blank. Each plate was incubated at 37° C. for the indicated timepoint (0, 10, 30, 60, 120 minutes). At the end of incubation, 400 μL of stop solution (200 ng/mL tolbutamide and 200 ng/mL labetalol in 0.1% FA in ACN) was added to precipitate protein. The plates were then sealed and shaken for 20 minutes before centrifugation at 4000 rpm for 20 minutes. 50 μL supernatant was transferred into 100 μL HPLC water and the bioanalysis plate sealed and shaken for 10 minutes prior to LC-MS/MS analysis (Shimadzu LC 30-AD, ACQUITY UPLC BEH C18 1.7 um 2.1*50 mm Column, Part No. 186002350, with mobile phase A: 0.1% formic acid in water, mobile phase B: 0.1% formic acid in acetonitrile). Formation of parent was also monitored as a metabolite.

The stability of the Examples in rat plasma is shown below in Table 4:

TABLE 4
Stability of Examples in rat plasma at 10 minute timepoint
is shown as % remaining and concentration (nM). Formation
of parent is also shown at the 10 minute timepoint.

| | | | | | Parent (Reference | |
|---|---|---|---|---|---|---|
| | % Example | | Example | | Example 1) | |
| Example | remaining | | concentration (nM) | | concentration (nM) | |
| No | 0 min | 10 min | 0 min | 10 min | 0 min | 10 min |
| 2 | ND | ND | ND | ND | ND | ND |
| 3 | 100.0 | 0.1 | 1830.9 | 2.7 | 36.0 | 1365.8 |
| 4 | ND | ND | ND | ND | ND | ND |
| 5 | ND | ND | ND | ND | ND | ND |
| 6 | 100.0 | 0.3 | 1779.1 | 5.0 | 79.5 | 1853.3 |
| 7 | 100.0 | 0.1 | 1887.9 | 1.2 | 205.5 | 1238.7 |
| 8 | 100.0 | 0.2 | 1687.0 | 3.1 | 34.3 | 1957.7 |
| 9 | ND | ND | ND | ND | ND | ND |
| 10 | 100.0 | 0.1 | 2023.5 | 2.1 | 158.8 | 1216.9 |
| 11 | ND | ND | ND | ND | ND | ND |
| 12 | ND | ND | ND | ND | ND | ND |
| 13 | 100.0 | 0.1 | 1601.0 | 1.6 | 40.9 | 2045.0 |
| 14 | ND | ND | ND | ND | ND | ND |
| 15 | ND | ND | ND | ND | ND | ND |
| 16 | 100.0 | 0.1 | 1845.9 | 2.7 | 42.4 | 1698.7 |
| 1 | 100.0 | 107.11 | | | | |

### Biological Assays for Characterization of Reference Example 1

### Assay 1: Biochemical Assay for Inhibitors of CK2α Kinase Activity

The inhibitory activity of putative kinase inhibitors and the potency of selected compounds were determined using ADP-Glo™ assay. The kinase reaction was performed in the presence of excess peptide substrate and ATP at a concentration equivalent to Kₘ. Upon termination of the kinase reaction, remaining ATP was depleted leaving only ADP reaction product, which was converted back to ATP with a coupled luciferin/luciferase reaction. The luminescent output from the coupled reaction was quantified and correlated with the kinase activity.

CK2α (residues 2-329) was produced in Escherichia coli BL21 (DE3) for kinase activity screening. Single colonies of the cells were grown in 6×1 L of 2×TY with 100 μg/mL ampicillin at 37° C. Isopropyl thio-R-D-galactopyranoside (IPTG) was added to a final concentration of 0.4 mM to induce expression when the optical density at 600 nm reached 0.6. The cells were incubated overnight at 25° C. then harvested by centrifugation at 4,000 g for 20 minutes. The cell pellets were suspended in 20 mM Tris, 500 mM NaCl, pH 8.0 and lysed using a high pressure homogenizer. Protease inhibitor cocktail tablets (one tablet per 50 mL extract; Roche Diagnostics) and DNase I were then added. The crude cell extract was then centrifuged at 10,000 g for 45 minutes, the supernatant was filtered with a 0.22 μm filter. The soluble supernatant was applied on a Ni Sepharose Fast Flow6 column at pH 8.0, washed and eluted in 20 mM Tris pH 8.0, 500 mM NaCl, 200 mM imidazole. After overnight dialysis into 20 mM Tris, pH 8.0, 500 mM NaCl the N-terminal His6-tag was cleaved overnight by TEV protease and passed through a second metal affinity column to remove uncleaved protein and the protease. The cleaved protein was further purified on a Sepharose Q HP anion-exchange column and the main peak fraction from this column was further purified by gel filtration on a Superdex 75 16/60 HiPrep column equilibrated with Tris 20 mM, pH 8.0, 500 mM NaCl. Pure protein was concentrated to 15 mg/mL and flash frozen in liquid nitrogen.

Final assay conditions comprised 0.2 nM CK2α, 50 μM peptide substrate (RRRADDSDDDDD), 15 μM ATP in 1× reaction buffer (40 mM Tris pH7.5, 200 mM NaCl, 20 mM MgCl₂, 0.1 mg/mL BSA, 1% DMSO). The assay was conducted as follows:
1. Appropriate serial dilutions of test compound were prepared using Echo (Labcyte) and 50 nL of 100× compound in 100% DMSO transferred to the assay plate (white opaque OptiPlate-384, Perkin-Elmer).
2. Enzyme and peptide substrate were prepared in fresh reaction buffer and added to the assay plate in a total volume of 3 μl and incubated at room temperature for 15 minutes.
3. 2 μL of ATP solution freshly prepared in reaction buffer was added to start the reaction.
4. After 120 minutes, the reaction was stopped by addition of 5 μl ADP-Glo reagent (Promega V9102) and the plate incubated at room temperature for a further 60 minutes.
5. 10 μL of Kinase Detection reagent (Promega V9102) was added to assay plate and incubated for a further 30 minutes prior to reading luminescence on an Envision (Perkin-Elmer).

Data was analysed to calculate compound IC₅₀ and K_{I} as follows:
1. All assay plates contained 32 wells designated as 0% inhibition control wells, which were treated with vehicle only (1% DMSO) and 32 wells designated as 100% inhibition control wells, which were treated with a high concentration of non-specific kinase inhibitor in 1% DMSO.
2. Percent inhibition in each test well was calculated using the formula (MEAN_{0% inhibition control wells}−test well reading)/(MEAN_{0% inhibition control wells}−MEAN_{100% inhibition control wells})×100%.
3. IC₅₀ was determined using a standard 4-parameter fit method (Model 205, XL-fit).
4. Percent activity was calculated for each well using: (Test well reading -MEAN_{100% inhibition control wells})/(MEAN_{0% inhibition control wells}−MEAN_{100% inhibition control wells}).
5. Morrison Kᵢ was determined using Morrison Kᵢ equation (XL-fit).

### Assay 2: Biochemical Assay for Inhibitors of CLK2 Kinase Activity

The assay was conducted in the same way as described for CK2α, with final assay conditions comprising 20 nM CLK2 (Carna Biosciences-04-127), 50 μM peptide substrate (KRRRLASLR), 100 μM ATP in 1× reaction buffer (40 mM Tris pH7.5, 200 mM NaCl, 20 mM MgCl2, 0.1 mg/mL BSA, 1% DMSO).

### Assay 3: Cell-Based NanoBRET™ Assay for Inhibitor Binding to Intracellular CK2α

This assay used the NanoBRET™ System (Promega), an energy transfer technique designed to measure molecular proximity in living cells. The assay measured the apparent affinity of test compounds by competitive displacement of a NanoBRET™ tracer reversibly bound to a NanoLucR luciferase CK2α fusion protein in cells. A fixed concentration of tracer was added to cells expressing the desired NanoLucR-CK2α fusion protein to generate a BRET reporter complex. Introduction of competing compounds resulted in a dose-dependent decrease in NanoBRET™ energy transfer, which allowed quantitation of the apparent intracellular affinity of the target protein for the test compound.

The assay was conducted as follows using HCT116 cell line (ATCC CCL-247™) transiently transfected with CSNK2A2-NanoLuc® Fusion Vector (Promega NV1191):
1. Cells were resuspended to 2×10⁵ cells/mL in Opti-MEM (Invitrogen 11058021).
2. DNA complex was prepared in a final volume of 1.4 ml Opti-MEM containing 15 μg DNA and 42 μl FuGENE HD Transfection reagent (Promega E2311).
3. 20 ml cell suspension was combined with 1 ml DNA complex, added to T75 flask and incubated overnight at 37° C. in 5% CO₂ incubator.
4. Appropriate serial dilutions of test compound were prepared and 5 μl/well transferred to the assay plate (white opaque CulturPlate-384, Perkin-Elmer) using Bravo (Agilent) with 5 μl NanoBRET Tracer K-5 (Promega N2501) diluted to the recommended concentration in assay buffer (Invitrogen 11058021) and 30 μl cell suspension. The plate was incubated for 2 hours at 37° C. in 5% CO₂ incubator.
5. 20 μl 3× complete substrate plus inhibitor solution (containing NanoBRET Nano-Glo substrate and extracellular NanoLuc inhibitor diluted to manufacturer's recommendations in assay medium) was added to each well.
6. Donor emission wavelength (450 nm) and acceptor emission wavelength (610 nm or 630 nm) were measured on the Envision (Perkin-Elmer) and BRET ratio calculated for data analysis: BRET Ratio=(Acceptorₛₐₘₚₗₑ/Donorₛₐₘₚₗₑ)×1,000.
7. All assay plates contained 32 wells designated as 0% inhibition control wells, which were treated with vehicle only (1% DMSO) and 32 wells designated as 100% inhibition control wells, which were treated with a high concentration of non-specific kinase inhibitor in 1% DMSO. Percent inhibition in each test well was calculated using the formula (MEAN_{0% inhibition control wells}−test well reading)/(MEAN_{0% inhibition control wells}−MEAN_{100% inhibition control wells})×100%.
8. IC₅₀ was determined using a standard 4-parameter fit method (Model 205, XL-fit).

### Biological Data:

| | | | Assay 3: |
|---|---|---|---|
| | Assay 1: | Assay 2: | NanoBRET ™ assay |
| | CK2α Enzyme | CLK2 Enzyme | for binding to |
| Example | IC₅₀ (nM) | IC₅₀ (nM) | intracellular CK2α |
| Reference | 0.32 | 346 | 5.56 |
| Example 1 | | | |

### REFERENCES

Battistutta & Lolli (2011). Structural and functional determinants of protein kinase CK2α: facts and open questions. Mol. Cell. Biochem., 2011, 356, 67-73.
Niefind et al (2001). Crystal structure of human protein kinase CK2: insights into basic properties of the CK2 holoenzyme. EMBO J. 2001, 20, 5320-5331.
Meggio & Pinna (2003). One-thousand-and-one substrates of protein kinas CK2. The FASEB Journal 17, 349-368.
Behan et al (2019). Prioritizaion of cancer therapeutic targets using CRISPR-Cas9 screens. Nature 568, 511-516.
Lin et al (2011). Overexpression of Nuclear Protein Kinase CK2α Catalytic Subunit (CK2α) as a Poor Prognosticator in Human Colorectal Cancer. PLoS ONE 6, 17193-Ortega et al (2014). Mining CK2 in cancer. PLoS ONE 9, 115609-
Di Maira et al. (2019). The protein kinase CK2 contributes to the malignant phenotype of cholangiocarcinoma cells. Oncogenesis 8, 61 P0 Zhan et al (2017). Wnt signaling in cancer. Oncogene 36, 1461-1473.
Gao & Wang (2006). Casein Kinase 2 is activated and essential for Wnt/0-Catenin signaling. Journal of Biological Chemistry 281, 189394-18400.
Dowling et al (2016). Potent and selective CK2 Kinase Inhibitors with effects on Wnt pathway signaling in vivo. ACS Med. Chem. Lett. 7, 300-305.
Zakharia et al. (2019). Pre-clinical in vitro and in vivo evidence of an antitumour effect of CX-4945, a casein kinase II inhibitor, in cholangiocarcinoma. Translational Oncology 12, 143-153.
Brear et al. (2016). Specific inhibition of CK2α from an anchor outside the active site. Chem. Sci. 7, 6839-6845.
Ruzzene & Pinna (2010). Addiction to protein kinase CK2: A common denominator of diverse cancer cells?Biochimica et Biophysica Acta 1804, 499-504.
Montenarh (2016). Protein Kinase CK2 in DNA Damage and repair. Transl. Cancer Res. 5, 49-63.
Gordon, D. E., Jang, G. M., Bouhaddou, M. et al. (2020). A SARS-CoV-2 protein interaction map reveals targets for drug repurposing. Nature 583, 459-468.

1. A compound, or pharmaceutically acceptable salt thereof, hydrate or solvate thereof, having the structural formula I shown below: 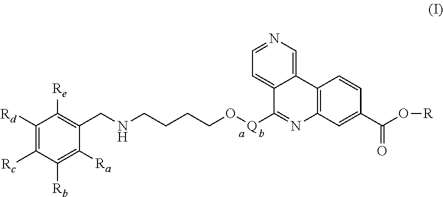
wherein:
R is an in vivo cleavable ester group;
Q is selected from formula Ia or Ib: 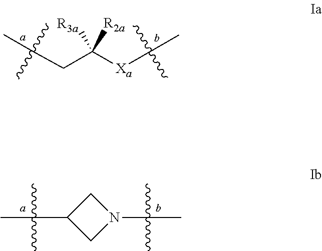
wherein:
bond a in formulae Ia and Ib corresponds with bond a in formula I and bond b in formulae Ia and Ib corresponds with bond b in formula I;
R₂ₐ and R₃ₐ are each independently selected from hydrogen or methyl; and
Xₐ is NH or O;
Rₐ and Rₑ are both independently selected from hydrogen, methyl or halo;
R_{b} and R_{d} are each independently selected from hydrogen, halo, cyano, (1-4C)alkyl,
—[CH₂]₀₋₃-(1-4C)alkoxy,
—[CH₂]₀₋₃—C(O)NH₂,
—[CH₂]₀₋₃—C(O)NH(1-4C)alkyl,
—[CH₂]₀₋₃—C(O)N[(1-4C)alkyl]₂,
—[CH₂]₀₋₃—NH(1-4C)alkyl,
—[CH₂]₀₋₃—N[(1-4C)alkyl]₂,
—[CH₂]₀₋₃—S(O)_{q}-(1-4C)alkyl (wherein q is 0, 1 or 2),
—[CH₂]₀₋₃—C(O)(1-4C)alkyl,
—[CH₂]₀₋₃—C(O)O-(1-4C)alkyl,
—[CH₂]₀₋₃—N(R_{f})C(O)-(1-4C)alkyl (wherein R_{f} is hydrogen or methyl),
—[CH₂]₀₋₃—S(O)₂NH(1-4C)alkyl,
—[CH₂]₀₋₃—S(O)₂N[(1-4C)alkyl]₂,
—[CH₂]₀₋₃—N(R)SO₂-(1-4C)alkyl (wherein R_{g} is hydrogen or methyl),
a group of the formula: 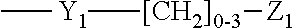
wherein Y₁ is absent, —O—, —NH—, —NMe—, —S—, —S(O)— or —S(O)₂—; and
Z₁ is (3-6C)cycloalkyl, phenyl, a 4- to 6-membered heterocyclyl or 5 or 6-membered heteroaryl;
and wherein:
any alkyl, alkoxy or any alkyl moiety within a R_{b} and R_{d} substituent group is optionally substituted by one or more substituents selected from halo, hydroxy, cyano, amino, —C(O)OH, —C(O)NH₂, (1-2C)alkoxy, or (3-4C)cycloalkoxy; and
Z₁ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl;
R_{c} is selected from hydrogen, halo, cyano, —C(O)NH₂, (1-4C)alkyl,
—[CH₂]₀₋₃-(1-4C)alkoxy,
—[CH₂]₀₋₃-(3-6C)cycloalkoxy,
—[CH₂]₀₋₃—C(O)NH₂,
—[CH₂]₀₋₃—C(O)NH(1-4C)alkyl,
—[CH₂]₀₋₃—C(O)N[(1-4C)alkyl]₂,
—[CH₂]₀₋₃—NH(1-4C)alkyl,
—[CH₂]₀₋₃—N[(1-4C)alkyl]₂,
—[CH₂]₀₋₃—S(O)_{q}-(1-4C)alkyl (wherein q is 0, 1 or 2),
—[CH₂]₀₋₃—C(O)(1-4C)alkyl,
—[CH₂]₀₋₃—C(O)O-(1-4C)alkyl,
—[CH₂]₀₋₃—N(Rₕ)C(O)-(1-4C)alkyl (wherein Rₕ is hydrogen or methyl),
—[CH₂]₀₋₃—S(O)₂NH(1-4C)alkyl,
—[CH₂]₀₋₃—S(O)₂N[(1-4C)alkyl]₂,
—[CH₂]₀₋₃—N(Rᵢ)SO₂-(1-4C)alkyl (wherein Rᵢ is hydrogen or methyl),
a group of the formula: 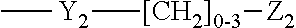
wherein Y₂ is absent, —O—, —NH—, —NMe—, —S—, —S(O)— or —S(O)₂—; and
Z₂ is (3-6C)cycloalkyl, phenyl, a 4- to 6-membered heterocyclyl or 5 or 6-membered heteroaryl;
and wherein:
any alkyl, alkoxy or any alkyl moiety within a R_{c} substituent group is optionally substituted by one or more substituents selected from halo, hydroxy, cyano, amino, —C(O)OH, —C(O)NH₂, (1-2C)alkoxy, or (3-4C)cycloalkoxy; and
Z₂ is optionally substituted by one or more substituents selected from: halo, hydroxy, cyano, amino, —C(O)OH, —C(O)NH₂, (1-2C)alkoxy, (1-2C)alkyl, (3-4C)cycloalkyl, (3-4C)cycloalkoxy, —C(O)NH(1-2C)alkyl, —C(O)N[(1-2C)alkyl]₂, —NH(1-2C)alkyl, —N[(1-2C)alkyl]₂, —S(O)_{q}-(1-2C)alkyl (wherein q is 0, 1 or 2), —C(O)(1-2C)alkyl, —C(O)O-(1-2C)alkyl, —N(R_{f})C(O)-(1-2C)alkyl, —S(O)₂NH(1-2C)alkyl, —S(O)₂N[(1-2C)alkyl]₂, or —NHSO₂-(1-2C)alkyl, and wherein any (1-2C)alkoxy, (1-2C)alkyl, (3-4C)cycloalkyl or (3-4C)cycloalkoxy group is optionally substituted by one or more substituents selected from halo, cyano, hydroxy, (1-2C)alkyl, (1-2C)alkoxy or (1-2C)alkoxy-(1-2C)alkyl.

2. A compound according to claim 1, or a pharmaceutically acceptable salt, hydrate or solvate thereof, with the proviso that the compound is not: (S)-Methyl 5-((1-(4-((3-chloro-4-(trifluoromethoxy)benzyl)amino)butoxy)propan-2-yl)oxy)benzo[c][2,6]naphthyridine-8-carboxylate.

3. The compound according to claim 1 or claim 2, or a pharmaceutically acceptable salt, hydrate or solvate thereof, wherein the compound has the structural formula II shown below: 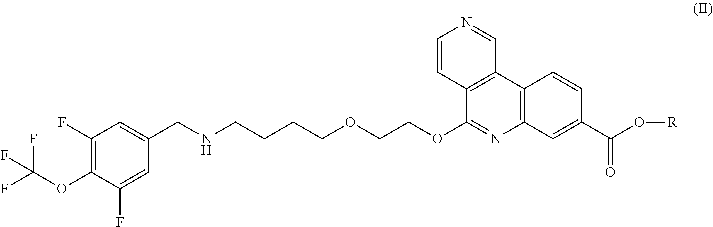
wherein R is an in vivo cleavable ester group.

4. The compound according to any one of the preceding claims, or a pharmaceutically acceptable salt, hydrate or solvate thereof, wherein R is selected from -(1-6C)alkyl, -(1-4C)alkylene-X—R₁, and -(1-4C)alkylene-R₂.

5. The compound according to any one of the preceding claims, or a pharmaceutically acceptable salt, hydrate or solvate thereof, wherein R is selected from -(1-4C)alkyl, -(1-2C)alkylene-X—R₁, and -(1-2C)alkylene-R₂.

6. The compound according to any one of the preceding claims, or a pharmaceutically acceptable salt, hydrate or solvate thereof, wherein R is selected from -methyl, -ethyl, -isopropyl, -tertbutyl, -methylene-X—R₁, -ethylene-X—R₁ and -methylene-R₂.

7. The compound according to any one of the preceding claims, or a pharmaceutically acceptable salt, hydrate or solvate thereof, wherein X is selected from —O—, —C(O)—, —C(O)O—, —O—C(O)—, —O—C(O)—O—, —CH(OR^{A})—, —N(R^{A})—, —N(R^{A})—C(O)—, —N(R^{A})—C(O)O—, —C(O)—N(R^{A})—, —N(R^{A})C(O)N(R^{A})—, —S—, —SO—, —SO₂—, —S(O)₂N(R^{A})—, or —N(R^{A})SO₂— wherein each R^{A} is independently selected from hydrogen or (1-6C)alkyl.

8. The compound according to any one of the preceding claims, or a pharmaceutically acceptable salt, hydrate or solvate thereof, wherein X is selected from —O—, —C(O)—, —C(O)O—, —O—C(O)—, —O—C(O)—O—, —N(R^{A})—, —N(R^{A})—C(O)— or —C(O)—N(R^{A})—, wherein R^{A} is selected from hydrogen or (1-3C)alkyl.

9. The compound according to any one of the preceding claims, or a pharmaceutically acceptable salt, hydrate or solvate thereof, wherein X is selected from —C(O)—, —C(O)O—, —O—C(O)—, —O—C(O)—O—, —N(H)— or —N(Me)—.

10. The compound according to any one of the preceding claims, or a pharmaceutically acceptable salt, hydrate or solvate thereof, wherein X is selected from —O—C(O)—, —O—C(O)—O— or —N(Me)—.

11. The compound according to any one of the preceding claims, or a pharmaceutically acceptable salt, hydrate or solvate thereof, wherein R₁ is selected from -hydrogen, -(1-6C)alkyl, -(2-6C)alkenyl, -(2-6C)alkynyl, -aryl, -(3-6C)cycloalkyl, -(3-6C)cycloalkenyl, -heteroaryl or -heterocyclyl.

12. The compound according to any one of the preceding claims, or a pharmaceutically acceptable salt, hydrate or solvate thereof, wherein R₁ is selected from -hydrogen, -(1-5C)alkyl, -aryl, -(3-6C)cycloalkyl, -(3-6C)cycloalkenyl, -heteroaryl or -heterocyclyl.

13. The compound according to any one of the preceding claims, or a pharmaceutically acceptable salt, hydrate or solvate thereof, wherein R₁ is selected from -hydrogen, -methyl, -ethyl, -propyl (e.g. n-propyl and iso-propyl), -butyl (e.g. n-butyl, iso-butyl, sec-butyl and tert-butyl), -phenyl, cyclopentane, cyclohexane, -5- or -6-membered heteroaryl or -5- or -6-membered heterocyclyl, wherein said heteroaryl or heterocyclyl comprises 1 or 2 heteroatoms selected from N, O and S.

14. The compound according to any one of the preceding claims, or a pharmaceutically acceptable salt, hydrate or solvate thereof, wherein R₁ is selected from -hydrogen, -methyl, -ethyl, -propyl (e.g. n-propyl and iso-propyl), -butyl (e.g. n-butyl, iso-butyl, sec-butyl and tert-butyl) or -6-membered heterocyclyl, wherein said heterocyclyl comprises 1 or 2 heteroatoms selected from N and O.

15. The compound according to any one of the preceding claims, or a pharmaceutically acceptable salt, hydrate or solvate thereof, wherein R₂ is selected from -aryl, -(3-6C)cycloalkyl, -(3-6C)cycloalkenyl, -heteroaryl or -heterocyclyl, wherein R₂ is optionally substituted by one or more substituents independently selected from -halo, -hydroxy, -cyano, -amino, -(1-4C)alkyl, -(1-4C)alkoxy, -oxo, —C(O)—R^{B}, —C(O)O—R^{B} or —C(O)—N(R^{B})₂, wherein each R^{B} is independently selected from hydrogen or (1-6C)alkyl.

16. The compound according to any one of the preceding claims, or a pharmaceutically acceptable salt, hydrate or solvate thereof, wherein R₂ is selected from -phenyl, -(4-6C)cycloalkyl, -(4-6C)cycloalkenyl, -5-, or -6-membered heteroaryl or -5-, or -6-membered heterocyclyl, wherein said heteroaryl or heterocyclyl comprises 1, 2 or 3 heteroatoms selected from N, O and S, wherein R₂ is optionally substituted by one or more substituents independently selected from -halo, -hydroxy, -cyano, -amino, -(1-3C)alkyl, -(1-3C)alkoxy, -oxo, —C(O)—R^{B}, —C(O)O—R^{B} or —C(O)—N(R^{B})₂, wherein each R^{B} is independently selected from hydrogen or (1-3C)alkyl.

17. The compound according to any one of the preceding claims, or a pharmaceutically acceptable salt, hydrate or solvate thereof, wherein R₂ is selected from −5-, or -6-membered heteroaryl or -5-, or -6-membered heterocyclyl, wherein said heteroaryl or heterocyclyl comprises 1 or 2 heteroatoms selected from N and O, wherein R₂ is optionally substituted by one, two or three substituents independently selected from -halo, -hydroxy, -cyano, -amino, -(1-3C)alkyl, -(1-3C)alkoxy, -oxo, —C(O)—H, —C(O)O—H or —C(O)—N(H)₂, —C(O)—Me, —C(O)O-Me or —C(O)—N(Me)₂.

18. The compound according to any one of the preceding claims, or a pharmaceutically acceptable salt, hydrate or solvate thereof, wherein R₂ is a dioxole (e.g. 1,2-dioxole or 1,3-dioxole), wherein R₂ is optionally substituted by two substituents independently selected from -halo, -hydroxy, -cyano, -amino, -(1-3C)alkyl, -(1-3C)alkoxy, -oxo, —C(O)—H, —C(O)O—H or —C(O)—N(H)₂, —C(O)—Me, —C(O)O-Me or —C(O)—N(Me)₂.

19. The compound according to any one of the preceding claims, or a pharmaceutically acceptable salt, hydrate or solvate thereof, wherein R₂ is 1,3-dioxole, wherein R₂ is substituted by one methyl group and one oxo group.

20. The compound according to any one of the preceding claims, or a salt, hydrate or solvate thereof, wherein the compound is selected from any one of the following:
isopropyl 5-(2-(4-((3,5-difluoro-4-(trifluoromethoxy)benzyl)amino)butoxy)ethoxy)benzo[c][2,6]naphthyridine-8-carboxylate;
2-(dimethylamino)ethyl 5-(2-(4-((3,5-difluoro-4-(trifluoromethoxy)benzyl)amino)butoxy)ethoxy)benzo[c][2,6]naphthyridine-8-carboxylate;
methyl 5-(2-(4-((3,5-difluoro-4-(trifluoromethoxy)benzyl)amino)butoxy)ethoxy)benzo[c][2,6]naphthyridine-8-carboxylate;
(isobutyryloxy)methyl 5-(2-(4-((3,5-difluoro-4-(trifluoromethoxy)benzyl)amino)butoxy)ethoxy)benzo[c][2,6]naphthyridine-8-carboxylate;
(pivaloyloxy)methyl 5-(2-(4-((3,5-difluoro-4-(trifluoromethoxy)benzyl)amino)butoxy)ethoxy)benzo[c][2,6]naphthyridine-8-carboxylate;
(5-methyl-2-oxo-1,3-dioxol-4-yl)methyl 5-(2-(4-((3,5-difluoro-4-(trifluoromethoxy)benzyl)amino)butoxy)ethoxy)benzo[c][2,6]naphthyridine-8-carboxylate;
ethyl 5-(2-(4-((3,5-difluoro-4-(trifluoromethoxy)benzyl)amino)butoxy)ethoxy)benzo[c][2,6]naphthyridine-8-carboxylate;
tert-butyl 5-(2-(4-((3,5-difluoro-4-(trifluoromethoxy)benzyl)amino)butoxy)ethoxy)benzo[c][2,6]naphthyridine-8-carboxylate;
acetoxymethyl 5-(2-(4-((3,5-difluoro-4-(trifluoromethoxy)benzyl)amino)butoxy)ethoxy)benzo[c][2,6]naphthyridine-8-carboxylate;
((methoxycarbonyl)oxy)methyl 5-(2-(4-((3,5-difluoro-4-(trifluoromethoxy)benzyl)amino)butoxy)ethoxy)benzo[c][2,6]naphthyridine-8-carboxylate;
(propionyloxy)methyl 5-(2-(4-((3,5-difluoro-4-(trifluoromethoxy)benzyl)amino)butoxy)ethoxy)benzo[c][2,6]naphthyridine-8-carboxylate;
((ethoxycarbonyl)oxy)methyl 5-(2-(4-((3,5-difluoro-4-(trifluoromethoxy)benzyl)amino)butoxy)ethoxy)benzo[c][2,6]naphthyridine-8-carboxylate;
((isopropoxycarbonyl)oxy)methyl 5-(2-(4-((3,5-difluoro-4-(trifluoromethoxy)benzyl)amino)butoxy)ethoxy)benzo[c][2,6]naphthyridine-8-carboxylate;
((tert-butoxycarbonyl)oxy)methyl 5-(2-(4-((3,5-difluoro-4-(trifluoromethoxy)benzyl)amino)butoxy)ethoxy)benzo[c][2,6]naphthyridine-8-carboxylate; and
((((tetrahydro-2H-pyran-4-yl)oxy)carbonyl)oxy)methyl 5-(2-(4-((3,5-difluoro-4-(trifluoromethoxy)benzyl)amino)butoxy)ethoxy)benzo[c][2,6]naphthyridine-8-carboxylate.

21. A pharmaceutical composition comprising a compound according to any one of claims 1 to 20, or a pharmaceutically acceptable salt, hydrate or solvate thereof, and a pharmaceutically acceptable excipient.

22. A compound according to any one of claims 1 to 20, or a pharmaceutically acceptable salt of solvate thereof, or a pharmaceutical composition according to claim 21 for use in:
(i) therapy:
(ii) the treatment of a disease or condition in which CK2α activity is implicated;
(iii) the treatment of a disease or condition associated with aberrant activity of CK2α;
(iv) the treatment of proliferative disorders (e.g. cancer or benign neoplasms), viral infections, an inflammatory disease or condition, diabetes, vascular and ischemic disorders, neurodegenerative disorders and/or the regulation of circadian rhythm;
(v) the treatment of a cancer; and/or
(vi) the treatment of a viral infection.
